(19) United States
(12) Patent Application Publication
(10) Pub. No.: US 2005/0038060 A1
(43) Pub. Date: Feb. 17, 2005

(54) Spiropoperidine compounds as ligands for orl-1 receptor

(76) Inventors: Koji Ando, Aichi-ken (JP); Fumitaka Ito, Aichi-ken (JP); Hiroki Koike, Aichi-ken (JP); Masaki Sudo, Aichi-ken (JP); Tatsuya Yamagishi, Aichi-ken (JP)

Correspondence Address: David R Kurlandsky;Warner Lambert Company, 2800 Plymouth Road, Ann Arbor, MI, 48105, US

(21) Appl. No.: 10/481,210

(22) PCT Filed: Jun. 17, 2002
(86) PCT No.: PCT/IB02/02272

Related U.S. Application Data
(60) Provisional application No. 60/301,079, filed on Jun. 26, 2001.

(51) Int. Cl. A61K 31/4747; C07D 471/10

(52) U.S. Cl. 514/278; 546/17

(57) ABSTRACT

A compound of the formula (I) or a salt, prodrug or solvate thereof, wherein R¹ and R² groups are all hydrogen; A is a benzofuzed azahetero ring; W¹—W² is CH₂—CH₂; X¹—X¹ is CH₂—CH₂; and Z is methylene or carbonyl; or the like, is a ligand for ORL1-receptor and are useful for treating or preventing pain, a CNS disorder or the like in mammalian subjects.

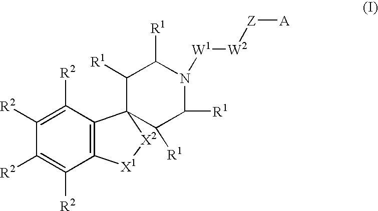

### TECHNICAL FIELD

This invention relates to substituted spiropiperidine compounds and their salts, prodrugs and solvates, and a medical use thereof. Also, this invention relates to a pharmaceutical composition comprising said compound, or its salt, prodrug or solvate. The compounds of this invention have binding affinity for ORL-1 receptor. In particular, compounds of this invention have selective antagonist activity for said receptor. The compounds of this invention are useful in treating or preventing disorders or medical conditions selected from pain, a CNS disorder and the like, which is mediated by said receptor and its endogeneous ligand.

### BACKGROUND ART

Three types of opioid receptors, μ (mu), δ (delta) and κ (kappa) have been identified. These receptors may be indicated with combinations of OP (abbreviation for Opioid Peptides) and numeric subscripts as suggested by the International Union of Pharmacology (IUPHAR). Namely, OP₁, OP₂ and OP₃ respectively correspond to δ-, κ- and μ-receptors. It has been found out that they belong to G-protein-coupled receptors and distribute in the central nervous system (CNS), peripheries and organs in a mammal. As ligands for the receptors, endogeneous and synthetic opioids are known. It is believed that an endogeneous opioid peptide produces their effects through an interaction with the major classes of opioid receptors. For example, endorphins have been purified as endogeneous opioid peptides and bind to both δ- and μ-receptors. Morphine is a well-known non-peptide opioid analgesic and has binding affinity mainly for μ-receptor. Opiates have been widely used as pharmacological agents, but drugs such as morphine and heroin induce some side effects such as drug addiction and euphoria.

Further, Meunier et al. reported isolation of a seventeen-amino-acid-long peptide from rat brain as an endogeneous ligand for an orphan opioid receptor (Nature, Vol. 337, pp. 532-535, Oct. 12, 1995). The receptor is known as “opioid receptor-like 1 (abbreviated as ORL1-receptor)” which is believed to be almost as homologous to any of μ-, δ- and κ-receptors. In the same report, the endogeneous opioid ligand has been introduced as agonist for ORL-1 receptor and named as “nociceptine (abbreviated as NC)”. Also, the same ligand was named as “orphanin FQ (abbreviated as OFQ or oFQ)” by Reinscheid et al. (Science, Vol. 270, pp. 792-794, 1995). This receptor may be indicated as OP₄ in line with a recommendation by IUPHAR in 1998 (British Journal of Pharmacology, Vol. 129, pp. 1261-1283, 2000).

Opioids and their affinity for these receptors have been researched in-vitro and in-vivo. It is possible to date to test whether an opioid has agonist or antagonist properties or a combination of both on the receptors.

Use of a synthetic ORL1-receptor ligand or antagonist as an analgesic is disclosed in WO 00/27815 (Smithkline Beecham Spa) or WO 99/48492 (Japan Tobacco Inc.).

Use of a synthetic ORL1-receptor antagonist for treating a CNS disorder is disclosed in WO 00/27815 (Smithkline Beecham Spa), WO 99/29696 (F. Hoffmann-La Roche A G) or British Journal of Pharmacology, Vol. 129, pp. 1261-1283, 2000 by G. Calo et al.

Banyu's WO 98/54168, WO 00/31061, WO 00/34280 and Japanese Patent Publication Kokai 2000-169476 disclose use of a synthetic ORL1-receptor ligand or antagonist as an analgesic or for treating a CNS disorder.

Schering's WO 01/07051 discloses use of a synthetic ORL-1 agonist in treating cough.

### BRIEF DISCLOSURE OF THE INVENSION

The present invention provides a compound of the following formula: 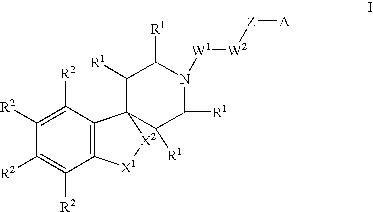
or pharmaceutically accptable salts thereof, wherein
each R¹ is independently selected from hydrogen and (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; or
two R¹ groups taken together form —CH₂— or CH₂)₂— and the remaining R¹ groups are defined as above;
each R² is independently selected from
   hydrogen; halo; hydroxy; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; non-, mono- and di-substituted amino wherein the substituents are independently selected from (C₁-C₆)alkyl, [(C₁-C₆)alkyl]-C(═O)—, [(C₁-C₆)alkoxy]-C(═O) and [(C₁-C₆)alkyl]-SO₂—; aryl selected from phenyl and naphthyl; and four- to eight-membered heterocyclyl containing one to four hetero atoms in the ring independently selected from nitrogen, oxygen and sulfur;
X¹ and X² are independently selected from
   (CH₂)ₙ₁ wherein n1 is an integer selected from 1, 2 and 3; C[(C₁-C₆)alkyl]; C—OH; O; NH; S; C(═O); SO₂; NR^{X1}; N—C(═O)R^{X2}; N—C(═O)OR^{X3}; and N—C(═O)NR^{X4}R^{X5}; wherein R^{X1}, R^{X2}, R^{X3}, R^{X4} and R^{X5} are independently (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; or
X¹ and X² taken together form CH═CH;
W¹ and W² are independently selected from CR^{W1}R^{W2}, wherein
   R^{W1} and R^{W2} are independently selected from hydrogen; halo; hydroxy; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO—;
      C(═O)-[(C₁-C₆)alkyl] wherein said (C₁-C₆)alkyl is optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; C(═O)—NR^{W11}R^{W12} wherein R^{W11} and R^{W12} are independently selected from hydrogen and (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; NR^{W13}R^{W14} wherein R^{W13} and R^{W14} are independently selected from hydrogen and (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O), [(C₁-C₆)alkoxy]-C(—O and [(C₁-C₆)alkyl]-SO₂—; aryl selected from phenyl and naphthyl; and four- to eight-membered heterocyclyl containing one to four hetero atoms independently selected from nitrogen, oxygen and sulfur;
A is selected from AA; AB; AC; AD and AE: 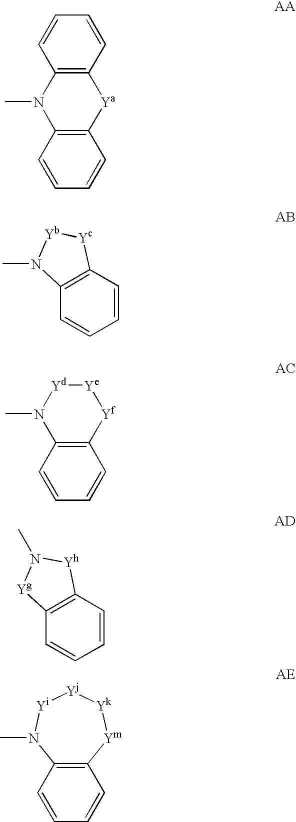
   wherein
   Y^{a} is selected from (CH₂)ₙ₂ wherein n2 is an integer selected from 0, 1 and 2; C(═O); NH; O and S;
   Y^{b}, Y^{c}, Y^{d}, Y^{e}, Y^{f}, Y^{g}, Y^{h}, Yⁱ, Y^{j}, Y^{k} and Y^{m} are independently selected from C(═O); CR^{Y1}R^{Y2}; CR^{Y3}[C(═O)R^{Y4}]; CR^{Y3}[NR^{Y5}C(═O)R^{Y4}]; CR^{Y3}[C(═O)NR^{Y6}R^{Y7}]; CR^{Y3}[NR^{Y6}R^{Y7}]; O; S; SO₂; NH; N[(C₁-C₆)alkyl] wherein said (C₁-C₆)alkyl is optionally substituted with one to three substituents independently selected from halo, hydroxy, carboxy, [(C₁-C₆)alkyl]-C(═O)—, (C₁-Cs)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; N—(CH₂)ₙ₃-heterocyclyl wherein n3 is an integer selected from 0, 1, 2 and 3, and said heterocyclyl contains from four to eight ring atoms one or two of which are independently selected from nitrogen, oxygen and sulfur, N—(CH₂)ₙ₄-aryl wherein n4 is an integer selected from 0, 1, 2 and 3, and said aryl is selected from phenyl and naphthyl; and N—(CH₂)ₙ₅-heteroaryl wherein n5 is an integer selected from 0, 1, 2 and 3, and said heteroaryl is a five to ten membered aromatic heterocyclyl containing from one to four hetero atoms independently selected from nitrogen, oxygen and sulfur, or
Y^{b} and Y^{c} taken together form a group selected from CR^{Y81}═CR^{Y82}; CR^{Y83}═N and N═N; and Y_{d}, Yₑ, Y^{f}, Y^{g} and Y^{h} are defined as above; wherein
   R^{Y1}, R^{Y2} and R^{Y5} are independently selected from hydrogen; hydroxy; non-, mono- and di-substituted amino wherein the substituents are independently selected from (C₁-C₆)alkyl; [(C₁-C₆)alkyl]-C(═O)—; [(C₁-C₆)alkoxy]-C(═O)—; [(C₁-C₆)alkyl]-SO₂—; and four- to eight-membered heterocyclyl containing one to four hetero atoms independently selected from nitrogen, oxygen and sulfur, wherein said heterocyclyl is optionally substituted with one to three substituents independently selected from hydroxy, (C₁-C₆)alkyl, NH₂—C(O═)—, [(C₁-C₆)alkyl]-NH—C(═O)—, [(C₁-C₆)alkyl]₂-N—C(═O)—, and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; or
   R^{Y1} and R^{Y2} taken together with the carbon atom to which they are attached form spiropyrrolidinyl or spiropiperidinyl, both of which are optionally N-substituted with a substituent selected from (C₁-C₆)alkyl, (C₁-C₆)alkyl-C(═O)—, [(C₁-C₆)alkyl]-C(═O)—(C₁-C₆)alkyl and aryl-(C═O)— wherein aryl is selected from phenyl and naphthyl; and R^{Y5} is defined as above;
   R^{Y3} is hydrogen;
   R^{Y4} is selected from hydroxy; (C₁-C₆)allyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(O)— and [(C₁-C₆)alkyl]-SO₂—; and
   R^{Y6} and R^{Y7} are independently selected from hydrogen; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; hetrocyclyl-(CH₂)ₙ₆— wherein n6 is an integer selected from 0, 1, 2, 3 and 4 and said heterocyclyl is four to eight membered containing one to three hetero atoms independently selected from nitrogen, oxygen and sulfur, wherein said heterocyclyl is optionally substituted with one to three substituents independently selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; and hetroaryl-(CH₂)ₙ₇— wherein n7 is an integer selected from 0, 1, 2, 3 and 4 and said heteroaryl is five to ten membered containing one to three hetero atoms independently selected from nitrogen, oxygen and sulfur, wherein said heteroaryl is optionally substituted with one to three substituents independently selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; or
   R^{Y6} and R^{Y7} taken together with the nitrogen atom to which they are attached form a four to eight heterocyclyl optionally containing, in addition to the nitrogen atom, one to two additional hetero atoms independently selected from nitrogen, oxygen and sulfur, and said heterocyclyl is optionally substituted with one substituent selected from hydroxy; (C₁-C₆)alkyl; NH₂—C(O═)—; (C₁-C₆)alkyl-NH—C(═O)—; [(C₁-C₆)alkyl]₂—N-C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—;
   R^{Y81}, R^{Y82} and R^{Y83} are independently selected from R^{Y811} and R^{Y812}C(═O)— wherein R^{Y811} and R^{Y812} are independently selected from hydrogen; hydroxy; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₅)alkyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and
said A is optionally substituted in the fused benzene rings with one to four substituents independently selected from halo; hydroxy; mercapto; phenyl; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a1}R^{a4}N—C(═O)—, wherein R^{a1}, R^{a2}, R^{a3} and R^{a4} are independently selected from hydrogen, (C₁C₆)alkyl, [(C₁-C₆)alkyl]-C(═O), [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O), wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and
Z is selected from C(═O); (CH₂)ₙ₈ wherein n8 is an integer selected from 0, 1 and 2; and CHR^{Z1} wherein R^{Z1} is selected from carboxy; (C₁-C₆)alkoxy-C(═O)—; non-, mono- and di-substituted amino wherein the substituents are independently selected from (C₁-C₆)alkyl, [(C₁-C₆)alkyl]-C(═O)—, [(C₁-C₆)alkyl]-C(═O)—O— and [(C₁-C₆)alkyl]-SO₂—; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and [C(═O)NR^{Z11}R^{Z12}] wherein R^{Z11} and R^{Z12} are independently selected from hydrogen and(C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—.

The compounds of the present invention have binding affinity for opioid receptor-like 1 (hereinafter referred to as “ORL-1 receptor”).

It is therefore an object of the present invention to provide a compound of formula I which is useful as a lignad for ORL-1 receptor.

It is another object of the present invention to provide a compound of formula I which is a modulator of ORL-1 receptor.

It is another object of the present invention to provide a compound of formula I having selective affinity for ORL-1 receptor. Preferably, these compounds have selective affinity for ORL-1 receptor than i-receptor.

It is another object of the present invention to provide a compound of formula I having antagonist activity for ORL-1 receptor.

It is another object of the present invention to provide a compound of formula I having selectivity for ORL-1 receptor and antagonist effect for said receptor.

The present invention relates to use of a compound of formula I as a ligand or a modulator for ORL-1 receptor, preferably as a selective ligand for said receptor, more preferably as an antagonist for said receptor, and most preferably as a selective antagonist for said receptor.

### DETAILED DESCRIPTION OF THE INVENTION

The term “pain” as used herein includes acute and chronic pain; neuropathic or inflammatory pain such as post herpetic neuralgia, neuralgia, diabetic neuropathy or post operative pain; osteoarthritis or back pain; pain in pregnancy labor and pains known to those skilled in the art (e.g., the pains described in Advances in Pain Research and Therapy, edited by C. R. Chapman et al., and published by Ravan Press (1989)).

The term “alkyl”, as used herein, means a straight or branched saturated monovalent hydrocarbon radical including, but not limited to, methyl, ethyl, propyl, isopropyl, n-butyl, sec-butyl, tert-butyl and the like.

The term “cycloalkyl”, as used herein, means a saturated carbocyclic radical including, but not limited to, cyclopropyl, cyclobutyl, cyclopentyl, cyclohexyl, cycloheptyl, cyclooctyl, cyclononyl, cyclodecyl and the like.

The term “alkoxy”, as used herein, means an O-alkyl group wherein “alkyl” is defined above.

The term “halo”, as used herein, refers to F, Cl, Br or I, preferably F or Cl.

The term “treating”, as used herein, refers to reversing, alleviating, inhibiting the progress of, or preventing the disorder or condition to which such term applies, or one or more symptoms of such disorder or condition. The term “treatment” as used herein refers to the act of treating, as “treating” is defined immediately above.

A preferred class of compound of formula (I) of this invention is that wherein:
all R¹ are hydrogen
each R² is independently selected from hydrogen and halo;
X¹ is selected from (CH²)ₙ₁ wherein n1 is an integer selected from 1, 2 and 3; O; NH;
S; C(═O); SO₂; and N[(C₁-C₄)alkyl];
X² is selected from CH₂; O; NH; S; C(═O); SO₂; and N[(C₁-C₄)alkyl]; or
X¹ and X² taken together form CH═CH;
W¹ and W² are independently selected from CR^{W1}R^{W2}, wherein
   R^{W1} and R^{W2} are independently selected from hydrogen; halo; hydroxy; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₅)alkyl]-C(═O)—, [(C₁-C₆)alkoxy]-C(═O)— and [(C₁-C₆)alkyl]-SO₂—; (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; C(═O)-[(C₁-C₆)alkyl] wherein said (C₁-C₆)alkyl is optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2, R}^{a3} and R^{a4} are independently selected from hydrogen, (C₁-C₆)alkyl, [(C₁-C₆)alkyl]-C(═O), [(C₁-C₆)alkoxy]-C(═O)— and [(C₁-C₆)alkyl]-SO₂—; C(═O)—NR^{W11}RW¹² wherein R^{W11} and R^{W12} are independently selected from hydrogen and (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2} R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; NR^{W13}R^{W14} wherein R^{W13} and R^{W14} are independently selected from hydrogen and (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; aryl selected from phenyl and naphthyl; and four- to eight-membered heterocyclyl containing one to four hetero atoms independently selected from nitrogen, oxygen and sulfur;
A is AB wherein
   Y^{b} and Y^{c} are independently selected from C(═O); CR^{Y1}R^{Y2}; CR^{Y3}[C(═O)R^{Y4}];
      CR^{Y3}[C(═O)NR^{Y6}R^{Y7}]; CR^{Y3}[NR^{Y6}R^{Y7}]; O; S; SO₂; NH; N[(C₁-C₆)alkyl] wherein said (C₁-C₆)alkyl is optionally substituted with one to three substituents independently selected from halo, hydroxy, carboxy, [(C₁-C₆)alkyl]-C(═O)—, (C₁-C₆)alkoxy, [(C₁-C₆alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; N—(CH₂)ₙ₃— heterocyclyl wherein n3 is an integer selected from 0, 1, 2 and 3, and said heterocyclyl contains from four to eight ring atoms one or two of which are independently selected from nitrogen, oxygen and sulfur, N—(CH₂)ₙ₄-aryl wherein n4 is an integer selected from 0, 1, 2 and 3, and said aryl is selected from phenyl and naphthyl; and N—(CH₂)ₙ₅-heteroaryl wherein n5 is an integer selected from 0, 1, 2 and 3, and said heteroaryl is a five to ten membered aromatic heterocyclyl containing from one to four hetero atoms independently selected from nitrogen, oxygen and sulfur, or
Y^{b} and Y^{c} taken together form a group selected from CR^{Y81}═CR^{Y82}; CR^{Y83}═N and N═N; and yd, ye, yf, Yg and yh are defined as above;
   R^{Y1} and R^{Y2} are independently selected from hydrogen; hydroxy; non-, mono- and di-substituted amino wherein the substituents are independently selected from (C₁-C₆)alkyl; [(C₁-C₆)alkyl]-C(═O)—; [(C₁-C₆)alkoxy]-C(═O)—; [(C₁-C₆)alkyl]-SO₂—; and four- to eight-membered heterocyclyl containing one to four hetero atoms independently selected from nitrogen, oxygen and sulfur, wherein said heterocyclyl is optionally substituted with one to three substituents independently selected from hydroxy, (C₁-C₆)alkyl, NH₂—C(O═)—, [(C₁-C₆)alkyl]-NH—C(═O)—, [(C₁-C₆)alkyl]₂-N—C(═O)—, and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6} R^{a7} and R^{a8} are independently selected from hydrogen, (C₁-C₆alkyl, [(C₁-C₆)alkyl]-C(═O)—, [(C₁-C₆)alkoxy]-C(═O) and [(C₁-C₆)alkyl]-SO₂—; or
      R^{Y1} and R^{Y2} taken together with the carbon atom to which they are attached form spiropyrrolidinyl or spiropiperidinyl, both of which are optionally N-substituted with a substituent selected from (C₁-C₆)alkyl, (C₁-C₆)alkyl-C(═O)—, [(C₁-C₆)alkyl]-C(═O)—(C₁-C₆)alkyl and aryl-(C═O)— wherein aryl is selected from phenyl and naphthyl;
   R^{Y3} is hydrogen;
   R^{Y4} is selected from hydroxy; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O), [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N_C(═O)_, wherein R^{a5}, R^{a6}, R^{a7} and R^{a8} are independently selected from hydrogen, (C₁-C₆)alkyl, [(C₁-C₆)alkyl]-C(═O), [(C₁-C₆)alkoxy]-C(═O)— and [(C₁-C₆)alkyl]-SO₂—; and
   R^{Y5}, R^{Y6} and R^{Y7} are independently selected from hydrogen; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; hetrocyclyl-(CH₂)ₙ₆— wherein n6 is an integer selected from 0, 1, 2, 3 and 4 and said heterocyclyl is four to eight membered containing one to three hetero atoms independently selected from nitrogen, oxygen and sulfur, wherein said heterocyclyl is optionally substituted with one to three substituents independently selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; and hetroaryl-(CH₂)ₙ₇— wherein n7 is an integer selected from 0, 1, 2, 3 and 4 and said heteroaryl is five to ten membered containing one to three hetero atoms independently selected from nitrogen, oxygen and sulfur, wherein said heteroaryl is optionally substituted with one to three substituents independently selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; or
   R^{Y6} and R^{Y7} taken together with the nitrogen atom to which they are attached form a four to eight heterocyclyl optionally containing, in addition to the nitrogen atom, one to two additional hetero atoms independently selected from nitrogen, oxygen and sulfur, and said heterocyclyl is optionally substituted with one substituent selected from hydroxy; (C₁-C₆)alkyl; NH₂—C(O═)—; (C₁-C₆)alkyl-NH—C(═O)—; [(C₁-C₆)alkyl]₂—N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(O)— and [(C₁-C₆)alkyl]-SO₂—;
   R^{Y81}, R^{Y82} and R^{Y83} are independently selected from R^{Y811} and R^{Y812}C(═O)— wherein R^{Y811} and R^{Y812} are independently selected from hydrogen; hydroxy; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and
      said A is optionally substituted in the fused benzene rings with one to four substituents independently selected from halo; hydroxy; mercapto; phenyl; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O) and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and
Z is selected from C(═O); (CH)ₙ₈ wherein n8 is an integer selected from 0, 1 and 2; and CHR^{Z1} wherein
   R^{Z1} is selected from carboxy; (C₁-C₆)alkoxy-C(═O)—; non-, mono- and di-substituted amino wherein the substituents are independently selected from (C₁-C₆)alkyl, [(C₁-C₆)alkyl]-C(═O)—, [(C₁-C₆)alkyl]-C(═O)—O— and [(C₁-C₆)alkyl]-SO₂—; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and
   [C(═O)—NR^{Z11}R^{Z12}] wherein R^{Z11} and R^{Z12} are independently selected from hydrogen and (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—.

A further preferred class of compound of formula (I) of this invention is that

wherein:

all R¹ are hydrogen
each R² is independently selected from hydrogen and halo;
X¹ is selected from (CH₂)ₙ₁ wherein n1 is an integer selected from 1, 2 and 3; O; NH;
S; C(═O); SO₂; and N[(C₁-C₄)alkyl];
X² is selected from CH₂; O; NH; S; C(═O); SO₂; and N[(C₁-C₄)alkyl]; or
X¹ and X² taken together form CH═CH;
W¹ and W² are both CH₂;
A is AB wherein
both Y^{b} and Y^{c} are independently selected from C(═O); CR^{R1}R^{Y2}; CR^{Y3}[C(═O)R^{Y4}];
CR^{Y3}[C(═O)NR^{Y6}R^{Y7}]; and CR^{Y3}[NR^{Y6}R^{Y7}], wherein
   R^{Y1} and R^{Y2} are independently selected from hydrogen; hydroxy; non-, mono- and di-substituted amino wherein the substituents are independently selected from (C₁-C₆)alkyl; [(C₁-C₆)alkyl]-C(═O)—; [(C₁-C₆)alkoxy]-C(═O)—; [(C₁-C₆)alkyl]-SO₂—; and four- to eight-membered heterocyclyl containing one to four hetero atoms independently selected from nitrogen, oxygen and sulfur, wherein said heterocyclyl is optionally substituted with one to three substituents independently selected from hydroxy, (C₁-C₆)alkyl, NH₂—C(O═)—, [(C₁-C₆)alkyl]-NH—C(═O)—, [(C₁-C₆)alkyl]₂-N—C(═O)—, and non-, mono- and di-substituted amino wherein the substituents are independently selected from (C₁-C₆)alkyl, [(C₁-C₆)alkyl]-C(═O)—, [(C₁-C₆)alkoxy]-C(O)— and [(C₁-C₆)alkyl]-SO₂—; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O) and [(C₁-C₆)alkyl]-SO₂—; or
   R^{Y1} and R^{Y2} taken together with the carbon atom to which they are attached form spiropyrrolidinyl or spiropiperidinyl, both of which are optionally N-substituted with a substituent selected from (C₁-C₆)alkyl, (C₁-C₆)alkyl-C(═O)—, [(C₁-C₆)alkyl]-C(═O)(C₁-C₆)alkyl and aryl-(C═O)— wherein aryl is selected from phenyl and naphthyl;
R^{Y3} is hydrogen;
R^{Y4} is selected from hydroxy; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O) and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)allyl, [(C₁-C₆)alkyl]-C(═O)—, [(C₁-C₆)alkoxy]-C(═O)— and [(C₁-C₆)alkyl]-SO₂—; and
R^{Y6} and R^{Y7} are independently selected from hydrogen; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═)—, [(C₁-C₆)alkoxy]-C(═O)— and [(C₁-C₆)alkyl]-SO₂—; hetrocyclyl-(CH₂)ₙ₆— wherein n6 is an integer selected from 0, 1, 2, 3 and 4 and said heterocyclyl is four to eight membered containing one to three hetero atoms independently selected from nitrogen, oxygen and sulfur, wherein said heterocyclyl is optionally substituted with one to three substituents independently selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; and hetroaryl-(CH₂)ₙ₇— wherein n7 is an integer selected from 0, 1, 2, 3 and 4 and said heteroaryl is five to ten membered containing one to three hetero atoms independently selected from nitrogen, oxygen and sulfur, wherein said heteroaryl is optionally substituted with one to three substituents independently selected from hydroxy; (C₁-C₆)alkyl; NH₂—C(O—)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-CE)alkyl]-C(═O)—, [(C₁-C₆)alkoxy]-C(═O)— and [(C₁-C₆)alkyl]-SO₂—; or
   R^{Y6} and R^{Y7} taken together with the nitrogen atom to which they are attached form a four to eight heterocyclyl optionally containing, in addition to the nitrogen atom, one to two additional hetero atoms independently selected from nitrogen, oxygen and sulfur, and said heterocyclyl is optionally substituted with one substituent selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—;
      said A is optionally substituted in the fused benzene rings with one to four substituents independently selected from halo; hydroxy; mercapto; phenyl; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}RN— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and
Z is selected from C(═O); (CH₂)ₙ₈ wherein n8 is an integer selected from 0, 1 and 2; and CHR^{Z1} wherein
   R^{Z1} is selected from carboxy; (C₁-C₆)alkoxy-C(═O)—; non-, mono- and di-substituted amino wherein the substituents are independently selected from (C, —C₆)alkyl, [(C₁-C₆)alkyl]-C(═O)—, [(C₁-C₆)alkyl]-C(═O)—O— and [(C₁-C₆)alkyl]-SO₂—; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and [C(═O)—NR^{Z11}R^{Z12}] wherein R^{Z11} and R^{Z12} are independently selected from hydrogen and (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁—C)alkyl]-SO₂—.

A further preferred class of compound of formula (I) of this invention is that wherein
all R¹ are hydrogen
each R² is independently selected from hydrogen and halo;
X¹ is selected from (CH₂)ₙ₁ wherein n1 is an integer selected from 1, 2 and 3; O; NH;
S; C(═O); SO₂; and N[(C₁-C₄)alkyl];
X² is selected from CH₂; O; NH; S; C(═O); SO₂; and N[(C₁-C₄)alkyl]; or
X¹ and X² taken together form CH═CH;
W¹ and W² are both CH₂;
A is AB wherein
Y^{b} is CR^{Y3}[C(═O)NR^{Y6}R^{Y7}]; and
Y^{c} is selected from CR^{Y1}R^{Y2}; CR^{Y3}[C(═O)R^{Y4}]; CR^{Y3}[C(═O)NR^{Y6}R^{Y7}]; and CR^{Y3}[NR^{Y6}R^{Y7}], wherein
   R^{Y1} and R^{Y2} are independently selected from hydrogen; hydroxy; non-, mono- and di-substituted amino wherein the substituents are independently selected from (C₁-C₆)alkyl; [(C₁-C₆)alkyl]-C(═O)—; [(C₁-C₆)alkoxy]-C(═O)—; [(C₁-C₆)alkyl]-SO₂—; and four- to eight-membered heterocyclyl containing one to four hetero atoms independently selected from nitrogen, oxygen and sulfur, wherein said heterocyclyl is optionally substituted with one to three substituents independently selected from hydroxy, (C₁-C₆)alkyl, NH₂—C(O═)—, [(C₁-C₆)alkyl]-NH—C(═O)—, [(C₁-C₆)alkyl]₂-N—C(═O)—, and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O) and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a1}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; or
   R^{Y1} and R^{Y2} taken together with the carbon atom to which they are attached form spiropyrrolidinyl or spiropiperidinyl, both of which are optionally N-substituted with a substituent selected from (C₁-C₆)alkyl, (C₁-C₆)alkyl-C(═O)—, [(C₁-C₆)alkyl]-C(═O)—(C₁-C₆)alkyl and aryl-(C═O)— wherein aryl is selected from phenyl and naphthyl;
R^{Y3} is hydrogen;
R^{Y4} is selected from hydroxy; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a1}N—C(═O)—, wherein R^{a5}, R^{a6}, R^{a7} and R^{a8} are independently selected from hydrogen, (C₁-C₆)alkyl, [(C₁-C₆)alkyl]-C(═O), [(C₁-C₆)alkoxy]-C(═O)— and [(C₁-C₆)alkyl]-SO₂—; and
R^{Y5}, R^{Y6} and R^{Y7} are independently selected from hydrogen; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; hetrocyclyl-(CH₂)ₙ₆— wherein n6 is an integer selected from 0, 1, 2, 3 and 4 and said heterocyclyl is four to eight membered containing one to three hetero atoms independently selected from nitrogen, oxygen and sulfur, wherein said heterocyclyl is optionally substituted with one to three substituents independently selected from hydroxy; (C₁-C₆)alkyl; NH₂—C(O═)—; (C₁-C₆)alkyl-NH—C(═O)—; [(C₁-C₆)alkyl]₂-N—C(═O)— and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; and hetroaryl-(CH₂)ₙ₇— wherein n7 is an integer selected from 0, 1, 2, 3 and 4 and said heteroaryl is five to ten membered containing one to three hetero atoms independently selected from nitrogen, oxygen and sulfur, wherein said heteroaryl is optionally substituted with one to three substituents independently selected from hydroxy; (C₁-C₆)alkyl; NH₂—C(O—)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; or
   R^{Y6} and R^{Y7} taken together with the nitrogen atom to which they are attached form a four to eight heterocyclyl optionally containing, in addition to the nitrogen atom, one to two additional hetero atoms independently selected from nitrogen, oxygen and sulfur, and said heterocyclyl is optionally substituted with one substituent selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—;
      said A is optionally substituted in the fised benzene rings with one to four substituents independently selected from halo; hydroxy; mercapto; phenyl; (C₁-C₆)alkyl optionally substituted with one to three substituents independently selected from halo, hydroxy, carboxy, (C₁-C₆)alkoxy, (C₁-C₆)alkoxy-C(═O)— and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, (C₁-C₆)alkoxy, (C₁-C₆)alkoxy-C(═O)— and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; and
Z is selected from C(═O); (CH₂)ₙ₈ wherein n8 is an integer selected from 0, 1 and 2; and CHR^{Z1} wherein
   R^{Z1} is selected from carboxy; (C₁-C₆)alkoxy-C(═O)—; non-, mono- and di-substituted amino wherein the substituents are independently selected from (C₁-C₆)alkyl, [(C₁-C₆)alkyl]-C(═O)—, [(C₁-C₆)alkyl]-C(═O)—O— and [(C₁-C₆)alkyl]-SO₂—; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and [C(═O)—NR^{Z11}R^{Z12}] wherein R^{Z11} and R^{Z12} are independently selected from hydrogen and (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—.

A further preferred class of compound of formula (I) of this invention is that wherein,
all R¹ are hydrogen
each R² is independently selected from hydrogen and halo;
X¹ is selected from (CH₂)ₙ₁ wherein n1 is an integer selected from 1, 2 and 3; O; NH;
S; C(═O); SO₂; and N[(C₁-C₄)alkyl];
X² is selected from CH₂; O; NH; S; C(═O); SO₂; and N[(C₁-C₄)alkyl]; or
X¹ and X² taken together form CH═CH;
W¹ and W² are both CH₂;
A is AB wherein
Y^{b} is CR^{Y3}[C(═O)NR^{Y6}R^{Y7}]; and
Y^{c} is selected from CR^{Y1}R^{Y2}; CR^{Y3}[C(═O)R^{Y4}]; CR^{Y3}[C(═O)NR^{Y6}R^{Y7}]; and
CR^{Y3}[NR^{Y6}N^{Y6}R^{Y7}]; wherein
   R^{Y1} and R^{Y2} are independently selected from hydrogen; hydroxy; non-, mono- and di-substituted amino wherein the substituents are independently selected from (C₁-C₆)alkyl; [(C₁-C₆)alkyl]-C(═O)—; [(C₁-C₆)alkoxy]-C(═O); [(C₁-C₆)alkyl]-SO₂—; and four- to eight-membered heterocyclyl containing one to four hetero atoms independently selected from nitrogen, oxygen and sulfur, wherein said heterocyclyl is optionally substituted with one to three substituents independently selected from hydroxy, (C₁-C₆)alkyl, NH₂—C(O═)—, [(C₁-C₆)alkyl]-NH—C(═O)—, [(C₁-C₆)alkyl]₂-N—C(═O)—, and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; or
      R^{Y1} and R^{Y2} taken together with the carbon atom to which they are attached form spiropyrrolidinyl or spiropiperidinyl, both of which are optionally N-substituted with a substituent selected from (C₁-C₆)alkyl, (C₁-C₆)alkyl-C(═O)—, [(C₁-C₆)alkyl]-C(═O)—(C₁-C₆)alkyl and aryl-(C═O)— wherein aryl is selected from phenyl and naphthyl;
   R^{Y3} is hydrogen;
   R^{Y4} is selected from hydroxy; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a5}N— and R^{a7}R^{a8}N—C(═O)—, wherein R^{a5}, R^{a5}, R^{a6} R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and
   R^{Y5}, R^{Y6} and R^{Y7} are independently selected from hydrogen; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; hetrocyclyl-(CH₂ₓ₎ₙ₆— wherein n6 is an integer selected from 0, 1, 2, 3 and 4 and said heterocyclyl is four to eight membered containing one to three hetero atoms independently selected from nitrogen, oxygen and sulfur, wherein said heterocyclyl is optionally substituted with one to three substituents independently selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; and hetroaryl-(CH₂)ₙ₇— wherein n7 is an integer selected from 0, 1, 2, 3 and 4 and said heteroaryl is five to ten membered containing one to three hetero atoms independently selected from nitrogen, oxygen and sulfur, wherein said heteroaryl is optionally substituted with one to three substituents independently selected from hydroxy; (C₁-C₆)alkyl; NH₂—C(O═)—; (C₁-C₆)alkyl-NH—C(═O)—; [(C₁-C₆)alkyl]₂-N—C(O)—; and non-, mono- and di-substituted amino wherein the substituents are independently selected from (C₁-C₆)allyl, [(C₁-C₆)alkyl]-C(═O)—, [(C₁-C₆)alkoxy]-C(═O)— and [(C₁-C₆)alkyl]-SO₂—; or
   R^{Y6} and R^{Y7} taken together with the nitrogen atom to which they are attached form a four to eight heterocyclyl optionally containing, in addition to the nitrogen atom, one to two additional hetero atoms independently selected from nitrogen, oxygen and sulfur, and said heterocyclyl is optionally substituted with one substituent selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—;
      said A is optionally substituted in the fused benzene rings with one to four substituents independently selected from halo; hydroxy; mercapto; phenyl; (C₁-C₆)alkyl optionally substituted with one to three substituents independently selected from halo, hydroxy, carboxy, (C₁-C₆)alkoxy, (C₁-C₆)alkoxy-C(═O)— and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, (C₁-C₆)alkoxy, (C₁-C₆)alkoxy-C(═O)— and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; and
Z is C(═O).

Individual preferred compounds of this invention include
2,3-dihydro-1′-{3-[2-(N-methylaminocarbonyl)indolin-1-yl]-3-oxopropyl}spiro[1H-indene-1,4′-piperidine];
2,3-dihydro-1′-[3-(2-N,N-dimethylaminocarbonylindolin-1-yl)-3-oxopropyl]spiro[1H-indene-1,4′-piperidine];
2,3-dihydro-1′-[3-(2-morpholinocarbonylindolin-1-yl)-3-oxopropyl]spiro[1H-indene-1,4′-piperidine];
2,3-dihydro-1′-[3-(2-carbamoylindolin-1-yl)-3-oxopropyl]spiro[1H-indene-1,4′-piperidine]hydrochloride;
2,3-dihydro-1′-{3-[2-(1-ethylprrolydin-3-yl)aminocarbonylindolin-1-yl]-3-oxopropyl}spiro[1H-indene-1,4′-piperidine];
2,3-dihydro-1′-{3-[2-(S)₄N,N-diethylaminoethyl)aminocarbonylindolin-1-yl]-3-oxopropyl} spiro[1H-indene-1,4′-piperidine];
2,3-dihydro-1′-{3-[2-(S(2-hydroxyethyl)aminocarbonylindolin-1-yl]-3-oxopropyl}spiro[1H-indene-1,4′-piperidine];
2,3-dihydro-1′-{3-[2-(S)-(2-aminoethyl)aminocarbonylindolin-1-yl]-3-oxopropyl}spiro[1H-indene-1,4′-piperidine];
2,3-dihydro-1′-{3-[2-(S)-(2-acetamidoethyl)aminocarbonylindolin-1-yl]-3-oxopropyl}spiro[1H-indene-1,4′-piperidine];
2,3-dihydro-1′-{3-[2-(S)-(2-methanesulfonamidoethyl)aminocarbonylindolin-1-yl]-3-oxopropyl}spiro[1H-indene-1,4′-piperidine];
2,3-dihydro-1′-[3-(2-(S)-N-methylaminocarbonylindolin-1-yl)-3-oxopropyl]spiro[1H-indene-1,4′-piperidine];
2,3-dihydro-1′-[3-(2-(S)-N,N-dimethylaminocarbonylindolin-1-yl)-3-oxopropyl]spiro[1H-indene-1,4′-piperidine];
2,3-dihydro-1′-{3-[2-(S)-(4-morpholinecarbonyl)indolin-1-yl]-3-oxopropyl}spiro[1H-indene-1,4′-piperidine]; and
2,3-dihydro-1′-[3-(2-(S)-aminocarbonylindolin-1-yl)-3-oxopropyl]spiro[1H-indene-1,4′-piperidine], or a salt thereof.

Another preferred class of compounds of formula (I) of this invention is that wherein
all R¹ are hydrogen
each R² is independently selected from hydrogen and halo;
X¹ is selected from (CH₂)ₙ₁, wherein n1 is an integer selected from 1, 2 and 3; O; NH;
S; C(═O); SO₂; and N[(C₁-C₄)alkyl];
X² is selected from CH₂; O; NH; S; C(═O); SO₂; and N[(C₁-C₄)alkyl]; or
X¹ and X² taken together form CH═CH;
W¹ and W² are both CH₂;
A is AB wherein
   Y^{b} is CR^{Y1}R^{Y2}; and
   Y^{c} is selected from CR^{Y1}R^{Y2}; CR^{Y3}[C(═O)R^{Y4}]; CR^{Y3}[C(═O)NR^{Y6}]; and CR^{Y3}[NR^{Y6}R^{Y7}]; or
   Y^{b} and Y^{c} taken together form a group selected from CH₂—CH₂ and CH₂═CH₂;
      R^{Y1} and R^{Y2} are independently selected from hydrogen; hydroxy; non-, mono- and di-substituted amino wherein the substituents are independently selected from (C₁-C₆)alkyl; [(C₁-C₆)alkyl]-C(═O)—; [(C₁-C₆)alkoxy]-C(═O}; [(C₁-C₆)alkyl]-SO₂—; and four- to eight-membered heterocyclyl containing one to four hetero atoms independently selected from nitrogen, oxygen and sulfur, wherein said heterocyclyl is optionally substituted with one to three substituents independently selected from hydroxy, (C₁-C₆)alkyl, NH₂—C(O═)—, [(C₁-C₆)alkyl]-NH—C(═O)—, [(C₁-C₆)alkyl]₂-N—C(═O)—, and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(O)—, R^{a1}R^{a2}N— and R^{a3}R^{a4}N—C(═O)—, wherein R^{a1}, R^{a2}, R^{a3}, and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; or
         R^{Y1} and R^{Y2} taken together with the carbon atom to which they are attached form spiropyrrolidinyl or spiropiperidinyl, both of which are optionally N-substituted with a substituent selected from (C₁-C₆)alkyl, (C₁-C₆)alkyl-C(—O)—, [(C₁-C₆)alkyl]-C(═O)—(C₁-C₆)alkyl and aryl-(C═O)— wherein aryl is selected from phenyl and naphthyl;
      R^{Y3} is hydrogen;
      R^{Y4} is selected from hydroxy; (C₁-C₆)alkyl optionally substituted with one to three substituents independently selected from halo, hydroxy, carboxy, [(C₁C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O), [(C₁-C₆alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a1}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and
      R^{Y6} and R^{Y7} are independently selected from hydrogen; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; hetrocyclyl-(CH₂)ₙ₆— wherein n6 is an integer selected from 0, 1, 2, 3 and 4 and said heterocyclyl is four to eight membered containing one to three hetero atoms independently selected from nitrogen, oxygen and sulfur, wherein said heterocyclyl is optionally substituted with one to three substituents independently selected from hydroxy; (C₁-C₆)alkyl; NH₂—C(O═)—; (C₁-C₆)alkyl-NH—C(═O)—; [(C₁-C₆)alkyl]₂—N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; and hetroaryl-(CH)ₙ₇— wherein n7 is an integer selected from 0, 1, 2, 3 and 4 and said heteroaryl is five to ten membered containing one to three hetero atoms independently selected from nitrogen, oxygen and sulfur, wherein said heteroaryl is optionally substituted with one to three substituents independently selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; or
         R^{Y6} and R^{Y7} taken together with the nitrogen atom to which they are attached form a four to eight heterocyclyl optionally containing, in addition to the nitrogen atom, one to two additional hetero atoms independently selected from nitrogen, oxygen and sulfur, and said heterocyclyl is optionally substituted with one substituent selected from hydroxy; [C₁-C₆)alkyl]; NH₂—C(O═)—; (C₁-C₆)alkyl-NH—C(═O)—; [(C₁-C₆)alkyl]₂—N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—;
            said A is optionally substituted in the fused benzene rings with one to four substituents independently selected from halo; hydroxy; mercapto; phenyl; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N_C(═O)_, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and
Z is C(═O).

Individual preferred compounds of this invention include
2,3-dihydro-1′-[3-(2-methoxycarbonylindolin-1-yl)-3-oxopropyl]spiro[1H-indene-1,4′-piperidine];
2,3-dihydro-1′-[3-(indolin-1-yl)-3-oxopropyl]spiro[1H-indene-1,4′-piperidine];
2,3-dihydro-1′-[3-(2-(S)-methoxycarbonylindolin-1-yl)-3-oxopropyl]spiro[1H-indene-1,4′-piperidine];
2,3-dihydro-1′-indolyl-3-oxopropylspiro[1H-indene-1,4′-piperidine];
2,3-dihydro-1′-[3-(2-hydroxymethylindolin-1-yl)-3-oxopropyl]spiro[1H-indene-1,4′-piperidine]; and
2,3-dihydro-1′-[3-(2-methoxymethylindolin-1-yl)-3-oxopropyl]spiro[1H-indene-1,4′-piperidine], or a salt thereof.

Another preferred class of compound of formula (I) is that wherein
all R¹ are hydrogen
each R² is independently selected from hydrogen and halo;
X¹ is selected from (CH₂)ₙ₁ wherein n1 is an integer selected from 1, 2 and 3; O; NH;
S; C(═O); SO₂; and N[(C₁-C₄)alkyl];
X² is selected from CH₂; O; NH; S; C(O); SO₂; and N[(C₁-C₄)alkyl]; or
X¹ and X² taken together form CH═CH;
W¹ and W² are both CH₂;
A is AB wherein
Y^{b} is selected from C(═O); CR^{Y1}R^{Y2}; CR^{Y3}[C(═O)R^{Y4}]; CR^{Y3}[NR^{Y5}C(═O)R^{Y4}];
CR^{Y3}[C(═O)NR^{Y6}R; and CR^{Y3}[NR^{Y6}R^{Y7}];
Y^{c} is selected from O; S; SO₂; NH; N[(C₁-C₆)alkyl] wherein said (C₁-C₆)alkyl is optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; N—(CH₂)ₙ₃-heterocyclyl wherein n3 is an integer selected from 0, 1, 2 and 3, and said heterocyclyl contains from four to eight ring atoms one or two of which are independently selected from nitrogen, oxygen and sulfur; N—(CH₂)ₙ₄-aryl wherein n4 is an integer selected from 0, 1, 2 and 3, and said aryl is selected from phenyl and naphthyl; and N—(CH₂)ₙ₅-heteroaryl wherein n5 is an integer selected from 0, 1, 2 and 3, and said heteroaryl is a five to ten membered aromatic heterocyclyl containing from one to four hetero atoms independently selected from nitrogen, oxygen and sulfur; wherein
   R^{Y1} and R^{Y2} are independently selected from hydrogen; hydroxy; non-, mono- and di-substituted amino wherein the substituents are independently selected from (C₁-C₆)alkyl; [(C₁-C₆)alkyl]-C(═O)—; [(C₁-C₆)alkoxy]-C(═O)—; [(C₁-C₆)alkyl]-SO₂—; and four- to eight-membered heterocyclyl containing one to four hetero atoms independently selected from nitrogen, oxygen and sulfur, wherein said heterocyclyl is optionally substituted with one to three substituents independently selected from hydroxy, (C₁-C₆)alkyl, NH₂—C(O═)—, [(C₁-C₆)alkyl]-NH—C(═O)—, [(C₁-C₆)alkyl]₂-N—C(═O)—, and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl)-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6, R}^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; or
      R^{a1} and R^{Y2} taken together with the carbon atom to which they are attached form spiropyrrolidinyl or spiropiperidinyl, both of which are optionally N-substituted with a substituent selected from (C₁-C₆)alkyl, (C₁-C₆)alkyl-C(═O)—, [(C₁-C₆)alkyl]-C(═O)—(C₁-C₆)alkyl and aryl-(C═O)— wherein aryl is selected from phenyl and naphthyl;
   R^{Y3} is hydrogen;
   R^{Y4} is selected from hydroxy; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2, R}^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a1} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and
      R^{Y5}, R^{Y6} and R^{Y7} are independently selected from hydrogen; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; hetrocyclyl-(CH₂)ₙ₆— wherein n6 is an integer selected from 0, 1, 2, 3 and 4 and said heterocyclyl is four to eight membered containing one to three hetero atoms independently selected from nitrogen, oxygen and sulfur, wherein said heterocyclyl is optionally substituted with one to three substituents independently selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; and
      hetroaryl-(CH₂)ₙ₇— wherein n7 is an integer selected from 0, 1, 2, 3 and 4 and said heteroaryl is five to ten membered containing one to three hetero atoms independently selected from nitrogen, oxygen and sulfur, wherein said heteroaryl is optionally substituted with one to three substituents independently selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; or
      R^{Y6} and R^{Y7} taken together with the nitrogen atom to which they are attached form a four to eight heterocyclyl optionally containing, in addition to the nitrogen atom, one to two additional hetero atoms independently selected from nitrogen, oxygen and sulfur, and said heterocyclyl is optionally substituted with one substituent selected from hydroxy; (C₁-C₆)alkyl; NH₂—C(O═)—; (C₁-C₆)alkyl-NH—C(═O)—; [(C₁-C₆)alkyl]; N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—;
         said A is optionally substituted in the fused benzene rings with one to four substituents independently selected from halo; hydroxy; mercapto; phenyl; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₉)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O), (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and
Z is selected from C(═O); (CH₂)ₙ₈ wherein n8 is an integer selected from 0, 1 and 2;
   and CHR^{Z1} wherein
      R^{Z1} is selected from carboxy; (C₁-C₆)alkoxy-C(═O)—; non-, mono- and di-substituted amino wherein the substituents are independently selected from (C₁-C₆)alkyl, [(C₁-C₆)alkyl]-C(═O)—, [(C₁-C₆)alkyl]-C(═O)—O— and [(C₁-C₆)alkyl]-SO₂—; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and [C(═O)—NR^{Z11}R^{Z12}] wherein R^{Z11} and R^{Z12} are independently selected from hydrogen and (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{z4}N—C(═O)—, wherein R^{z1}, R^{a2}, R^{a3} and R^{z4} are independently selected from hydrogen, (C₁-C₆)alkyl, [(C₁-C₆)alkyl]-C(═O)—, [(C₁-C₆)alkoxy]-C(═O) and [(C₁-C₆)alkyl]-SO₂—.

Individual preferred compounds of this invention include
2,3-dihydro-1′-[3-(benzimidazol-2-one-1-yl)propyl]spiro[1H-indene-1,4′-piperidine];
2,3-dihydro-1′-[3-(benzothiazol-2-one-1-yl)propyl]spiro[1H-indene-1,4′-piperidine];
2,3-dihydro-1′-[3-(2-oxo-1,3-benzoxazol-3(2H)-yl)propyl]spiro[1H-indene-1,4′-piperidine];
2,3-dihydro-1′-[3-(2-hydroxymethylbenzimidazol-1-yl)₃-oxopropyl]spiro[1H-indene 1,4′-piperidine];
2,3-dihydro-1′-[3-(3-ethylbenzimidazol-2-one-1-yl)propyl]spiro[1H-indene-1,4′-piperidine];
2,3-dihydro-1′-[3-(2-acetamidobenzimidazol-1-yl)propyl]spiro[1H-indene-1,4′-piperidine];
2,3-dihydro-1′-{3-[3-(2-hydroxyethyl)benzimidazol-2-one-1-yl]propyl}spiro[1H-indene-1,4′-piperidine];
2,3-dihydro-1′-{3-[3-(2-aminoethyl)benzimidazol-2-one-1-yl)propyl} spiro[1H-indene-1,4′-piperidine]; and
2,3-dihydro-1′-{3-[3-(2-acetamidoethyl)benzimidazol-2-one-1-yl]propyl}spiro[1H-indene-1,4′-piperidine], or a salt thereof.

Another preferred class of compound of formula (I) of this invention is that wherein
all R¹ are hydrogen
each R² is independently selected from hydrogen and halo;
X¹ is selected from (CH₂)ₙ₁ wherein n1 is an integer selected from 1, 2 and 3; O; NH;
S; C(═O); SO₂; and N[(C₁-C₄)alkyl];
X² is selected from CH₂; O; NH; S; C(═O); SO₂; and N[(C₁-C₄)alkyl]; or
X¹ and X² taken together form CH═CH;
W¹ and W² are independently selected from CR^{W1}R^{W2},
   wherein
   R^{W1} and R^{W2} are independently selected from hydrogen; halo; hydroxy; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a2}R^{a6}N— and R^{a7}R^{a8}N—C(O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; C(═O)—[(C₁-C₆)alkyl]wherein said (C₁-C₆)alkyl is optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; C(═O)—NR^{W11}R^{W12} wherein R^{W11} and R^{W12} are independently selected from hydrogen and (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; NR^{W13}R^{W14} wherein R^{W13} and R^{W14} are independently selected from hydrogen and (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{z3} and R^{z4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; aryl selected from phenyl and naphthyl; and four- to eight-membered heterocyclyl containing one to four hetero atoms independently selected from nitrogen, oxygen and sulfur;
A is AC wherein
   Y_{d}, Y^{e} and Y^{f} are independently selected from C(═O); CR^{Y1}R^{Y2}; CR^{Y3}[C(═O)R^{Y4}]; CR^{Y3}NR^{Y5}C(═O)R^{Y4}; CR^{Y3}[C(═O)NR^{Y6}R^{Y7}]; CR^{Y3}[NR^{Y6}R^{Y7]}; O; S; SO₂; NH; N[(C₁-C₆)alkyl] wherein said (C₁-C₆)alkyl is optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; N—(CH₂)ₙ₃-heterocyclyl wherein n3 is an integer selected from 0, 1, 2 and 3, and said heterocyclyl contains from four to eight ring atoms one or two of which are independently selected from nitrogen, oxygen and sulfur N—(CH₂)ₙ₄-aryl wherein n4 is an integer selected from 0, 1, 2 and 3, and said aryl is selected from phenyl and naphthyl; and N—(CH₂)ₙ₅-heteroaryl wherein n5 is an integer selected from 0, 1, 2 and 3, and said heteroaryl is a five to ten membered aromatic heterocyclyl containing from one to four hetero atoms independently selected from nitrogen, oxygen and sulfur,
      R^{Y1} and R^{Y2} are independently selected from hydrogen; hydroxy; non-, mono- and di-substituted amino wherein the substituents are independently selected from (C₁-C₆)alkyl; [(C₁-C₆)alkyl]-C(═O)—; [(C₁-C₆)alkoxy]-C(═O)—; [(C₁-C₆)alkyl]-SO₂—; and four- to eight-membered heterocyclyl containing one to four hetero atoms independently selected from nitrogen, oxygen and sulfur, wherein said heterocyclyl is optionally substituted with one to three substituents independently selected from hydroxy, (C₁-C₆)alkyl, NH₂—C(O═)—, [(C₁-C₆)alkyl]-NH—C(═O)—, [(C₁-C₆)alkyl]₂—N—C(═O)—, and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and (C₁-Cs)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; or
         R^{Y1} and R^{Y2} taken together with the carbon atom to which they are attached form spiropyrrolidinyl or spiropiperidinyl, both of which are optionally N-substituted with a substituent selected from (C₁-C₆)alkyl, (C₁-C₆)alkyl-C(═O)—, [(C₁-C₆)alkyl]-C(═O)—(C₁-C₆)alkyl and aryl-(C═O)— wherein aryl is selected from phenyl and naphthyl;
      R^{Y3} is hydrogen;
      R^{Y4} is selected from hydroxy; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and
      R^{Y5}, R^{Y6} and R^{Y7} are independently selected from hydrogen; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a1}, R^{a2} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; hetrocyclyl-(CH₂)ₙ₆— wherein n6 is an integer selected from 0, 1, 2, 3 and 4 and said heterocyclyl is four to eight membered containing one to three hetero atoms independently selected from nitrogen, oxygen and sulfur, wherein said heterocyclyl is optionally substituted with one to three substituents independently selected from hydroxy; (C₁-C₆)alkyl; NH₂—C(O═)—; (C₁-C₆)alkyl-NH—C(═O)—; [(C₁-C₆)alkyl]₂—N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O) and [(C₁-C₆)alkyl]-SO₂—; and hetroaryl-(CH₇)ₙ₇— wherein n7 is an integer selected from 0, 1, 2, 3 and 4 and said heteroaryl is five to ten membered containing one to three hetero atoms independently selected from nitrogen, oxygen and sulfur, wherein said heteroaryl is optionally substituted with one to three substituents independently selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; or
         R^{Y6} and R^{Y7} taken together with the nitrogen atom to which they are attached form a four to eight heterocyclyl optionally containing, in addition to the nitrogen atom, one to two additional hetero atoms independently selected from nitrogen, oxygen and sulfur, and said heterocyclyl is optionally substituted with one substituent selected from hydroxy; (C₁-C₆)alkyl; NH₂—C(O═)—; (C₁-C₆)alkyl-NH—C(═O)—; [(C₁-C₆)alkyl]₂—N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; and
            said A is optionally substituted in the fused benzene rings with one to four substituents independently selected from halo; hydroxy; mercapto; phenyl; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and
Z is selected from C(═O); (CH₂)ₙ₈ wherein n8 is an integer selected from 0, 1 and 2; and CHR^{Z1} wherein
   R^{Z1} is selected from carboxy; (C₁-C₆)alkoxy-C(═O)—; non-, mono- and di-substituted amino wherein the substituents are independently selected from (C₁-C₆)alkyl, [(C₁-C₆)alkyl]-C(═O)—, [(C₁-C₃alkyl]-C(═O)—O— and [(C₁-C₆)alkyl]-SO₂—; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and [C(═O)_NR^{Z11}R^{Z12}] wherein R^{Z11} and R^{Z12} are independently selected from hydrogen and (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—.

Individual preferred compounds of this invention include 2,3-dihydro-1′-[3-(2-oxo-3,4-dihydro-[(2H)-quinolinyl)propyl]spiro[1H-indene-1,4′-piperidine] and 2,3-dihydro-1′-[3-(3-methyl-2-oxo-3,4-dihydro-[(2H)-quinazolinyl)propyl]spiro[1H-indene-1,4′-piperidine]; or a salt thereof.

Another preferred class of compound of formula (I) of this invention is that wherein
all R¹ are hydrogen
each R² is independently selected from hydrogen and halo;
X¹ is selected from (CH₂)ₙ₁ wherein n1 is an integer selected from 1, 2 and 3; O; NH;
S; C(O); SO₂; and N[(C₁-C₄)alkyl];
X² is selected from CH₂; O; NH; S; C(═O); SO₂; and N[(C₁-C₄)alkyl]; or
X¹ and X² taken together form CH═CH;
W¹ and W² are independently selected from CR^{W1}R^{W2},
   wherein
   R^{W1} and R^{W2} are independently selected from hydrogen; halo; hydroxy; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N and R^{a7}R^{a8}N_C(O)_, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; C(═O)—[(C₁-C₆)alkyl] wherein said (C₁-C₆)alkyl is optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a2} and R^{a4} are independently selected from hydrogen, (C₁-C₆)alkyl, [(C₁-C₆alkyl)]-C(═O)—, [(C₁-C₆)alkoxy]-C(═O)— and [(C₁-C₆)alkyl]-SO₂—; C(═O)—NR^{W1}R^{W12} wherein R^{W11} and R^{W12} are independently selected from hydrogen and (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O), wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; NR^{W13}R^{W14} wherein R^{W13} and R^{W14} are independently selected from hydrogen and (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; aryl selected from phenyl and naphthyl; and four- to eight-membered heterocyclyl containing one to four hetero atoms independently selected from nitrogen, oxygen and sulfur;
A is AE wherein
   Yⁱ, Y^{j}, Y^{k} and Y^{m} are independently selected from C(═O); CR^{Y1}R^{Y2}; CR^{Y3}[C(═O)R^{Y4}]; CR^{Y3}[NR^{Y5}C(═O)R^{Y4}]; CR^{Y3}[C(═O)NR^{Y6}R^{Y7}]; CR^{Y3}[NR^{Y6}R^{Y7}]; O; S; SO₂; NH; N[(C₁-C₆)alkyl] wherein said (C₁-C₆)alkyl is optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{z4} are independently selected from hydrogen, (C₁-C₆)alkyl, [(C₁-C₆)alkyl]-C(═O)—, [(C₁-C₆)alkoxy]-C(═O) and [(C₁-C₆)alkyl]-SO₂—; N—(CH₂)ₙ₃-heterocyclyl wherein n3 is an integer selected from 0, 1, 2 and 3, and said heterocyclyl contains from four to eight ring atoms one or two of which are independently selected from nitrogen, oxygen and sulfur; N—(CH₂)ₙ₄-aryl wherein n4 is an integer selected from 0, 1, 2 and 3, and said aryl is selected from phenyl and naphthyl; and N—(CH₂)ₙ₅— heteroaryl wherein n5 is an integer selected from 0, 1, 2 and 3, and said heteroaryl is a five to ten membered aromatic heterocyclyl containing from one to four hetero atoms independently selected from nitrogen, oxygen and sulfur,
      R^{Y1} and R^{Y2} are independently selected from hydrogen; hydroxy; non-, mono- and di-substituted amino wherein the substituents are independently selected from (C₁-C₆)alkyl; [(C₁-C₆)alkyl]-C(═O)—; [(C₁-C₆)alkoxy]-C(═O)—; [(C₁-C₆)alkyl]-SO₂—; and four- to eight-membered heterocyclyl containing one to four hetero atoms independently selected from nitrogen, oxygen and sulfur, wherein said heterocyclyl is optionally substituted with one to three substituents independently selected from hydroxy, (C₁-C₆)alkyl, NH₂—C(O═)—, [(C₁-C₆)alkyl]-NH—C(═O)—, [(C₁-C₆)alkyl]₂—N—C(═O)—, and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O}, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; or
         R^{Y1} and R^{Y2} taken together with the carbon atom to which they are attached form spiropyrrolidinyl or spiropiperidinyl, both of which are optionally N-substituted with a substituent selected from (C₁-C₆)alkyl, (C₁-C₆)alkyl-C(═O)—, [(C₁-C₆)alkyl]-C(═O)—(C₁-C₆)alkyl and aryl-(C═O)— wherein aryl is selected from phenyl and naphthyl;
      R^{Y3} is hydrogen;
      R^{Y4} is selected from hydroxy; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N_C(═O)_, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and
      R^{Y5}, R^{Y6} and R^{Y7} are independently selected from hydrogen; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; hetrocyclyl-(CH₆)ₙ₆— wherein n6 is an integer selected from 0, 1, 2, 3 and 4 and said heterocyclyl is four to eight membered containing one to three hetero atoms independently selected from nitrogen, oxygen and sulfur, wherein said heterocyclyl is optionally substituted with one to three substituents independently selected from hydroxy; (C₁-C₆)alkyl; NH₂—C(O═)—; (C₁-C₆)alkyl-NH—C(═O)—; [(C₁-C₆)alkyl]2-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; and hetroaryl-(CH₂)ₙ₇— wherein n7 is an integer selected from 0, 1, 2, 3 and 4 and said heteroaryl is five to ten membered containing one to three hetero atoms independently selected from nitrogen, oxygen and sulfur, wherein said heteroaryl is optionally substituted with one to three substituents independently selected from hydroxy; (C₁-C₆)alkyl; NH₂—C(O═)—; (C₁-C₆)allyl-NH—C(═O)—; [(C₁-C₆)alkyl]₂—N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; or
      R^{Y6} and R^{Y7} taken together with the nitrogen atom to which they are attached form a four to eight heterocyclyl optionally containing, in addition to the nitrogen atom, one to two additional hetero atoms independently selected from nitrogen, oxygen and sulfur, and said heterocyclyl is optionally substituted with one substituent selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; and
         said A is optionally substituted in the fused benzene rings with one to four substituents independently selected from halo; hydroxy; mercapto; phenyl; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O), R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O), wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and
Z is selected from C(═O); (CH₂)ₙ₈ wherein n8 is an integer selected from 0, 1 and 2; and
CHR^{Z1} wherein
   R^{Z1} is selected from carboxy; (C₁-C₆)alkoxy-C(═O)—; non-, mono- and di-substituted amino wherein the substituents are independently selected from (C₁-C₆)alkyl, [(C₁-C₆)alkyl]-C(═O)—, [(C₁-C₆)alkyl]-C(═O)—O— and [(C₁-C₆)alkyl]-SO₂—; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O), wherein R^{a1}, R^{a2}, R^{a3} and R^{a4} are independently selected from hydrogen, (C₁-C⁶)alkyl, [(C₁-C₆)alkyl]-C(═O)—, [(C₁-C₆)alkoxy]-C(═O)— and [(C₁-C₆)alkyl]-SO₂—; and [C(═O)—NR^{Z11}R^{Z12}] wherein R^{Z11} and R^{Z12} are independently selected from hydrogen and (C₁-C₆)alkyl optionally substituted with one to three substituents independently selected from halo, hydroxy, carboxy, [(C₁-C₆)alkyl]-C(═O), (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—.

Individual preferred compounds of this invention include 2,3-dihydro-1′-[3-oxo-3-(2,3,4,5-tetrahydro-1H-benzazepin-1-yl)propyl]spiro[1H-indene-1,4′-piperidine] or a salt thereof.

Another preferred class of compounds of this invention is that wherein
all R¹ are hydrogen
each R² is independently selected from hydrogen and halo;
X¹ and X² are independently selected from the group consisting of C[(C₁-C₆)alkyl] and
C—OH;
W¹ and W² are both CH₂;
A is AB wherein
Y^{b} is selected from C(═O); CR^{Y1}R^{Y2}; CR^{Y3}[C(═O)R^{Y4}]; CR^{Y3}[NR^{Y5}C(═O)R^{Y4}]; CR^{Y3}[C(═O)NR^{Y6}R^{Y7}]; and CR^{Y3}[NR^{Y6}R^{Y7}];
Y^{c} is selected from O; S; SO₂; NH; N[(C₁-C₆)alkyl] wherein said (C₁-C₆)alkyl is optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; N—(CH₂)ₙ₃-heterocyclyl wherein n3 is an integer selected from 0, 1, 2 and 3, and said heterocyclyl contains from four to eight ring atoms one or two of which are independently selected from nitrogen, oxygen and sulfur; N—(CH₂)ₙ₄-aryl wherein n4 is an integer selected from 0, 1, 2 and 3, and said aryl is selected from phenyl and naphthyl; and N—(CH₂)ₙ₅-heteroaryl wherein n5 is an integer selected from 0, 1, 2 and 3, and said heteroaryl is a five to ten membered aromatic heterocyclyl containing from one to four hetero atoms independently selected from nitrogen, oxygen and sulfur; wherein
   R^{Y1} and R^{Y2} are independently selected from hydrogen; hydroxy; non-, mono- and di-substituted amino wherein the substituents are independently selected from (C₁-C₆)alkyl; [(C₁-C₆)alkyl]-C(═O)—; [(C-C₆)alkoxy]-C(═O); [(C₁-C₆)alkyl]-SO₂—; and four- to eight-membered heterocyclyl containing one to four hetero atoms independently selected from nitrogen, oxygen and sulfur, wherein said heterocyclyl is optionally substituted with one to three substituents independently selected from hydroxy, (C₁-C₆)alkyl, NH₂—C(O═)—, [(C₁-C₆)alkyl]-NH—C(═O)—, [(C₁-C₆)alkyl]₂-N—C(═O)—, and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; or
      R^{Y1} and R^{Y2} taken together with the carbon atom to which they are attached form spiropyrrolidinyl or spiropiperidinyl, both of which are optionally N-substituted with a substituent selected from (C₁-C₆)alkyl, (C₁-C₆)alkyl-C(═O)—, [(C₁-C₆)alkyl]-C(═O)—(C₁-C₆)alkyl and aryl-(C═O)— wherein aryl is selected from phenyl and naphthyl;
   R^{Y3} is hydrogen;
      R^{Y4} is selected from hydroxy; (C₁-C₆)allyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O), R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O), [(C₁-C₆)alkoxy]-C(═O)— and [(C₁-C₆)alkyl]-SO₂—; and
   R^{Y5}, R^{Y6} and R^{Y7} are independently selected from hydrogen; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{z4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; hetrocyclyl-(CH₂)ₙ₆— wherein n6 is an integer selected from 0, 1, 2, 3 and 4 and said heterocyclyl is four to eight membered containing one to three hetero atoms independently selected from nitrogen, oxygen and sulfur, wherein said heterocyclyl is optionally substituted with one to three substituents independently selected from hydroxy; (C₁-C₆)allyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; and hetroaryl-(CH₂)ₙ₇— wherein n7 is an integer selected from 0, 1, 2, 3 and 4 and said heteroaryl is five to ten membered containing one to three hetero atoms independently selected from nitrogen, oxygen and sulfur, wherein said heteroaryl is optionally substituted with one to three substituents independently selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; or
      R^{Y6} and R^{Y7} taken together with the nitrogen atom to which they are attached form a four to eight heterocyclyl optionally containing, in addition to the nitrogen atom, one to two additional hetero atoms independently selected from nitrogen, oxygen and sulfur, and said heterocyclyl is optionally substituted with one substituent selected from hydroxy; (C₁-C₆)alkyl; NH₂—C(O═); (C₁-C₆)alkyl-NH—C(═O)—; [(C₁-C₆)alkyl]₂-N—C(═O); and non-, mono- and di-substituted amino wherein the substituents are independently selected from (C₁-C₆)alkyl, [(C₁-C₆)alkyl]-C(═O)—, [(C₁-C₆)alkoxy]-C(O)—and [(C₁-C₆)alkyl]-SO₂—;
         said A is optionally substituted in the fused benzene rings with one to four substituents independently selected from halo; hydroxy; mercapto; phenyl; (C₁-C₆)alkyl optionally substituted with one to three substituents independently selected from halo, hydroxy, carboxy, [(C₁-C₆)alkyl]-C(═O), (C₁-C₆)alkoxy, [(C₁-C₆)alkoxy]-C(═O)—, R^{a1}R^{a2}N— and R^{a3}R^{a4}N—C(═O)—, wherein R^{a1}, R^{z2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and
Z is selected from C(═O); (CH₂)ₙ₈, wherein n8 is an integer selected from 0, 1 and 2; and
CHR^{Z1} wherein
   R^{Z1} is selected from carboxy; (C₁-C₆)alkoxy-C(═O)—; non-, mono- and di-substituted amino wherein the substituents are independently selected from (C₁-C₆)alkyl, [(C₁-C₆)alkyl]-C(═O)—, [(C₁-C₆)alkyl]-C(═O)—O— and [(C₁-C₆)alkyl]-SO₂—; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆alkyl, [(C₁-C₆)alkyl]-C(═O)—, [(C₁-C₆)alkoxy]-C(═O)— and [(C₁-C₆)alkyl]-SO₂—; and [C(═O)—NR^{Z11}R^{Z12}] wherein R^{Z11} and R^{A12} are independently selected from hydrogen and (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—.

Individual preferred compounds of this invention include 1′-[3-[(2S)-2-[(dimethylamino)carbonyl]-2,3-dihydro-1H-indol-1-yl]-3-oxopropyl]spiro[(2-hydroxy)indane-1,4′-piperidine] and 1′-[3-[(2S)₂-[(Dimethylamino)carbonyl]-2,3-dihydro-1H-indol-1-yl]-3-oxopropyl]spiro[(3-methyl)indane-1,4′-piperidine] or a salt thereof.

Accordingly, this invention relates to a pharmaceutical composition comprising an effective amount of a compound of formula I defined as above and a pharmaceutically acceptable carrier for treating a disease or medical condition mediated by ORL1-receprot and its endogeneous ligand in a mammal including a human.

A preferred pharmaceutical composition of this invention comprises a compound of formula I defined as above having selectivity for ORL-1 receptor.

A further preferred pharmaceutical composition of this invention comprises a compound of formula I defined as above having antagonist effect for ORL-1 receptor.

A further preferred pharmaceutical composition of this invention comprises a compound of formula I defined as above which is a selective antagonist for ORL-1 receptor.

Therefore, a pharmaceutical composition of this invention comprising a compound of formula I defined as above is useful for treating or preventing a disease or medical condition selected from pain; eating disorders including anorexia and bulimia; anxiety and stress conditions; immune system diseases; locomotor disorder; eating disorder; memory loss, cognitive disorders and dementia including senile dementia and those diseases caused by Alzheimer's disease, Perkinson's disease or other neurodegenerative pathologies; epilepsy or convulsion and symptoms associated therewith; a central nervous system disorder related to glutamate release action, anti-epileotic action, disruption of spatial memory, serotonin release, anxiolytic action, mesolimbic dopaminergic transmission, rewarding propaerties of drug of abuse, modulation of striatal and glutamate effects on locomotor activity; cardiovascular disorders hypotension, bradycardia and stroke; renal disorders including water excretion, sodium ion excretion and syndrome of inappropriate secretion of antidiuretic hormone (SIADH); gastrointestinal disoders; airway disorders including adult respiratory distress syndrome (ARDS); autonomic disorders including suppression of micturition reflex; metabolic disorders including obesity; cirrhosis with ascites; sexsual dysfunctions; and altered pulmonary function including obstructive pulmonary disease.

This invention also relates to a method for treating or preventing a disease or condition in a mammal including a human, which disease or condition is mediated by ORL-1 receptor and its endogeneous ligand, comprising administering an effective amount of a compound of formula I defined as above to a mammal including a human, which suffered from such disease or condition.

More specifically, this invention relates to a method for treating or preventing the aforementioned disease or medical condition, wherein said compound has selectivity for ORL-1 receptor.

More specifically, this invention relates to a method of treating or preventing the aforementioned disease or medical condition, wherein said compound has antagonist effect for ORL-1 receptor.

More specifically, this invention relates to a method for treating or preventing the aforementioned disease or medical condition, wherein said compound is a selective antagonist for ORL-1 receptor.

Accordingly, this invention relates to a method for treating or preventing the aforementioned disease or medical condition wherein said disease or condition is selected from pain; eating disorders including anorexia and bulimia; anxiety and stress conditions; immune system diseases; locomotor disorder; eating disorder; memory loss, cognitive disorders and dementia including senile dementia and those diseases caused by Alzheimer's disease, Perkinson's disease or other neurodegenerative pathologies; epilepsy or convulsion and symptoms associated therewith; a central nervous system disorder related to gulutamate release action, anti-epileotic action, disruption of spatial memory, serotonin release, anxiolytic action, mesolimbic dopaminergic transmission, rewarding propaerties of drug of abuse, modulation of striatal and glutamate effects on locomotor activity; cardiovascular disorders hypotension, bradycardia and stroke; renal disorders including water excretion, sodium ion excretion and syndrome of inappropriate secretion of antidiuretic hormone (SIADH); gastrointestinal disoders; airway disorders including adult respiratory distress syndrome (ARDS); autonomic disorders including suppression of micturition reflex; metabolic disorders including obesity; cirrhosis with ascites; sexsual dysfunctions; and altered pulmonary function including obstructive pulmonary disease.

General Synthesis:

The compounds of formula I of the present invention may be prepared according to known preparation methods, or General Procedures or preparation methods illustrated in the following reaction Schemes. Unless otherwise indicated R¹, R², X¹, X², W¹, W², A and Z, and groups or substituents thereof, in the reaction Schemes and discussion that follow are defined as above. Unless otherwise indicated, reactions in this specification may be earred out at about ambient pressure (i.e., 760 mmHg) and about room temperature (i.e., 25° C.).

Typical preparation procedures for compounds of formula I of the present invention are as follow:

Protecting Groups:

Amino, hydroxy, mercapto or the like may be protected with a protecting group, and the protectinng group may be subsequently removed in an appropriate reaction step according to a known procedure (e.g., Protective Groups in Organic Synthesis edited by T. W. Greene et al. (John Wiely & Sons, 1991)). For example, a primary or a secondary amine may be typically protected by reaction with benzyl chloride in K₂CO₃ solution, and the benzyl group (abbreviated as Bn) may be removed by catalytic hydrogenation over palladium-carbon. Introduction for t-butoxycarbonyl (abbreviated as Boc) to amino group may be carried out using (BOC)₂O under basic condition, and the protecting group may be removed in HCl/EtOAc. Hydroxy may protected with t-butyldimethylsilyl (abbreviated as TBS or TBDMS) in alkylation using NaH. The protecting group may be introduced with TBDMSCl in imidazole and DMF and removed using an appropriate reagent such as tetrabutylammonium fluoride.

Leaving Groups/Introductions of Sulfonyl Groups:

Leaving group used in a reaction described hereafter are known to those skilled in the art. These leaving groups include halo such as C₁, Br and I; sulfonic esters such as TfO (triflates), MsO (mesylates), TsO (tosylates); and the like. These groups may be introduced to an appropriate compound according to methods known to those skilled in the art (e.g., (a) halogenation using triphenylphosphine/CX₄ wherein X is halo (PPh₃/CX₄); (b) reaction with TsCl; and (c) reaction with MsCl).

Halogenations:

Halogenations may be used for displacement of hydroxy group by a halogen atom. These halogenations are typically carried out using halogenating reagents such as hydrogen halogenide (e.g., HCl, HBr or HI), sulfinyl halogenide (e.g., SOCl₂ or SOBr₂), phosphorous halides (PCl₃, PCl, PBr₃ or PBrs), phosphoryl chloride (POCl₃), Ph₃PCl₂, Ph₃P—CCl₄ system, a combination of N-bromosuccinimide (NBS) or 1,3-dibromo-5,5-dimethylhydanton with Ph₃P in DMF, Ph₃PBr₂, system of Ph₃P-diethyl azodicarboxylate-hydroxy commpound-LiBr, trimethylsilyl bromide (Me₃SiBr) or trimethylsilyl chloride (Me₃SiCl) and LiBr, white or red phosphorous and I₂, diphosphorous tetraiodide (P214), trimethylsilyl iodide (Me₃SiI) and sodium iodide (NaI), trimethylsilyl polyphosphate (PPSE), a fluorobenzothiazolium or fluoropyridinium salt, carbodiimidinium iodide or the like. If appropriate, these halogenations may be carried out in a reaction inert solvent such as DMF, hexamethylphosphoric triamide (HMPA), or the like. These halogenations may be typically carried out at a temperature from about 0° C. to about the reflux temperature of the reaction mixture from about 1 minutes to about 10 hours.

Alkylations:

Alkylations may be carried out according to a procedure known to those skilled in the art. More specifically, a primary or secondary amine may be alkylated to a secondary or tertialy amine with a halo alkyl in the presence of an alkali metal ion such as potassium ion, base or a mixture thereof. This alkylation may be also carried out using a nucleophilic strong base that serves to remove the proton of the secondary amine radical. Instead of halides, sulfates or sulfonates may be used in these reactions. Alkylations of alcohols may be carried out using diazo compounds preferably in the presence of a catalyst such as fluoboric acid (HBF₄) or silica gel. For the alkylations, suitable solvents include polar aprotic solvents such as dimethylformamide (DMF), dimethylsulfoxide, acetonitrile (MeCN), acetone, sulfur dioxide, dichloromethane, hexane and the like; and protic solvents such as water, alcohols such as methanol (MeOH) and ethanol (EtOH), ethylene glycol and the like, or a combination thereof. These reactions may be typically carried out at a temperature from about 0° C. to the reflux temperature of a solvent to be used for from about 1 minute to 30 hours.

Michael Reaction may be carried out in the presence of a base. Suitable bases for this reaction include NaOC₂H₅, KOH, KOC(CH₃)₃, triethylamine (Et₃N), NaH, BuLi, lithium diisopropylamide (LDA) and the like.

Alkylation of cyclic amines may be carried out using metal hydride reagents. Suitable hydride reagents for this reaction include borohydrides such as NaBH₄, NaBH(OAc)₃ and NaBH₃CN. This reaction may be preferably carried out under mildly acidic conditions. For example, alkylation of a cyclic amine with an aldehyde or ketone compound may be typically carried out using NaBH(OAc)₃ or NaBH₃CN and an acid such as acetic acid or HCl in a reaction inert solvent such as CH₂Cl₂, an alcohol (e.g., MeOH, EtOH or i-PrOH), THF, MeCN or the like.

Aminations:

Aminations of alkanols or alkyl halides may be carried out by reactions with cyclic imide compounds such as N-phthalimides followed by hydrazinolysis or hydrolysis. If required, the reactions with phthalimides may be carried out using organophosphorous reagents with or without azo compounds.

Amidations:

Amidation 1—Dehydration of Ammonium Salts:

Amidations of carboxylic acids and amines may be carried out at elevated temperatures. This reaction may be catalyzed by acid or by cation exchange resin.

Amidation 2—Acylayion of Amines by Acyl Halides:

Acyl halids may be treated with ammonia or amines for the preparation of amides. This reaction is usually carried out in the presence of a base such as triethylamine or potassium carbonate to take up the evolving hydrogen halide. If appropriate, a coupling agent such as carbodiimide may be used. The reaction temperature may be controlled by cooling or dilution. Acyl halide may also be reacted with arylamines, hydrazine or hydroxylamine under the similar conditions. Amino protections using carbobenzoxy group (abbreviated as Cbz) or t-butoxycarbonyl group (abbreviated as Boc) may be carried out in this way.

Amidation 3—Acylation of Amines by Carboxylic Acid Anhydrides:

This reaction may be carried out with ammonia or primary or secondary amines according to a similar procedure for acylation of amines by acyl halides.

Amidation 4—Acylation of Amines by Carboxylic Acids:

Carboxylic acids may be treated with ammonia or amine compounds to give amides. This amidation may be carried out in the presence of a coupling agent with or without an additional base at about room temperature. Suitable coupling agents include carbodiimides such as dicyclohexylcarbodiimide (DCC) used in a peptide synthesis. Other suitable coupling agents used in these amidations include N,N′-carbonyldiimidazole (CDI), diisopropylcarbodiimide (DIPC), 1-ethyl-3-(3-dimethylaminopropyl)carbodiimide (WSC, water soluble carbodiimide), benzotriazole-1-yloxy-tris(dimethylamino)phosphonium hexafluorophosphate (BOP), diphenylphosphorylazide (DPPA) and the like. A cyclic amine may be acylated according to a method analogous to these amidations. If amines are subjected to this reaction in its halogen salt forms, additional amines may be used for trapping hydrogen halides formed.

Amidation 5—Acylation of Amines by Carboxylic Esters:

Carboxylic esters may be converted to unsubstituted, N-substituted or N,N-disubstituted amides. This reaction may be carried out in the presence of a strong base catalysis as well as catalysis by cyanide ion under a high pressure. Hydrazides and hydroxamic acids may be prepared from carboxylic esters with hydrazine and hydroxylamine respectively under similar reaction conditions.

Amidation 6—Acylation of Amines by Amides or Other Acid Derivatives:

A salt of an amine may be subjected to this reaction. In this reaction, NH₂ usually acts as a leaving group. Secondary and primary amines (in the form of their salts) are the most common reagents in this reaction. Acid derivatives, which may be converted to amides, include thiol acids, thio]ethers, acyloxyboranes, 1,1,1-trihalo ketones, α-keto nitrils, acyl azides and the like.

These amidations may be carried out in a reaction inert solvent such as dichloromethane (CH₂Cl₂), alcohols such as methanol, ethanol or buthanol (BtOH), acetonitrile, tetrahydrofiran (THF), dimethyfuran (DMF), or pyridine or a combination thereof, at a temperature from about 0° C. to the reflux temperature of a solvent, for from about 5 minutes to 48 hours.

Hydrolysis of Esters:

Hydrolysis of esters may be carried out in the presence of an acid, base, metal ion, enzyme or nucleophile according to a method known to those skilled in the art. The hydrolysis of esters may be carried out in a reaction inert solvent at a temperature from about 0° C. to the reflux temperature of the solvent for from about 1 to 24 hours. Suitable solvents for the reactions include alcohols such as methanol, ethanol, tetrahydrofuran, acetic acid and the like.

Esterifications:

Carboxylic acids and alcohols afford esters using acid catalysis. Typical catalysis for this reaction include conc. HCl, anhydrous sulfuric acid, p-toluenesulfonic acid and the like. The alcohol generally servers as the solvent, but other reaction inert solvent such as toluene or xylene may be used. The alcohol may be used in large excess, and the water from the reaction mixture may be removed.

Reductions:

Reductions may be carried out using reducing agents such as hydride reagents. Typical reducing regents are lithium aluminum hydride (LiAlH₄), lithium triethylborohydride (LiEt₃BH), lithium trialkoxyaluminum hydride (e.g., LiAlH(OMe)₃ and LiAlH(OBu-tert)₃), LiAlH₄-AlCl₃, diisobutylaluminum hydride (DIBAL-H), NaBH₄, NaBH(OAC)₃, Me₄NBH(OAc)₃, NaBH₃CN, LiBH₄, LiR₃BH, [(C₂Hs)₃SiH], B₂H₆, dialkylboron (R₂BH) or the like. Other reducing agents are zinc with acid or base, SnCl₂, chromium(II) ion and the like. This reaction may be carried out in an inert solvent at a temperature from about −78° C. to about the reflux temperature of the solvent. For example, reduction using LiAiH₄ may be carried out in tetrahydrofiran, and reduction using NaBH₄ may be carried out in an alcohol such as methanol (MeOH) or ethanol (EtOH).

Schemes 1-1,1-2 and 1-3 illustrate embodiments of preparation process for a compound of formula (I).

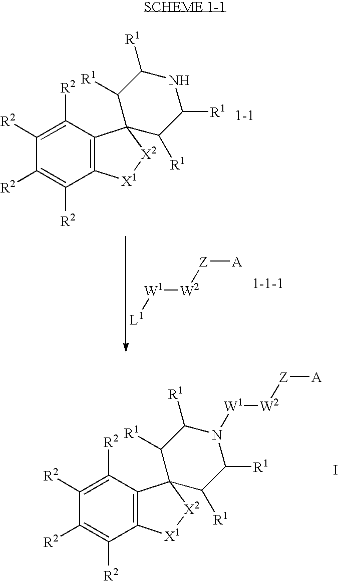

Scheme 1-1 illustrates a preparation method of a compound of formula I of the present invention. This method comprises alkylation of a spiro-piperidine compound of formula 1-1 by a compound of formula 1-1-1 wherein L¹ is a leaving group. This reaction may be carried out according to an alkylation of an amine compound. In a preferred embodiment of this reaction, a compound of formula 1-1 may be used as potassium salt, then reacted with a compound of formula 1-1-1 wherein the leaving group L¹ may be halo. The potassium salt of a compound formula 1-1 may be prepared by treating said compound with a potassium salt such as potassium carbonate, potassium hydroxide or a combination thereof. The following alkylation may be carried out at an elevated temperature, for example at about the reflux temperature of a reaction inert solvent used. Typically, this reaction may be carried out in acetonitrile (MeCN) using potassium carbonate (K₂CO₃) and potassium iodide (KI).

Scheme 1-2 illustrates another preparation method of a compound of formula (I).

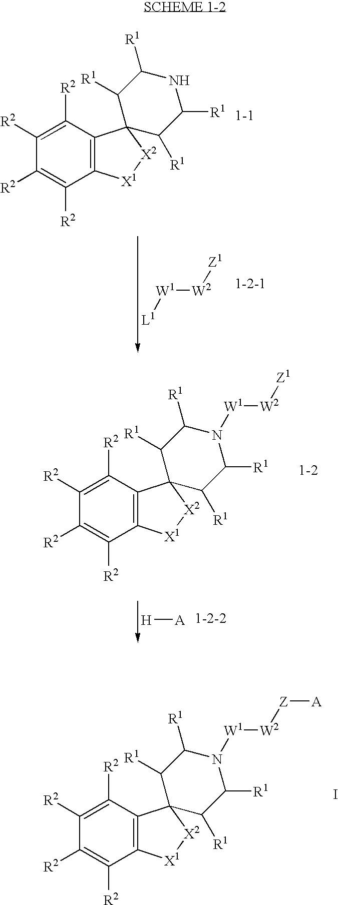

A compound of formula I may be prepared from a compound of formula 1-1 by alkylation with a compound of formula 1-2-1 followed by an amination with a compound of formula 1-2-2. In formula 1-2-1, Z¹ is Z as defined in formula (1) or its analogous group comprising a leaving group, carbonyl, hydroxy or carboxy; and L¹ is a leaving group similar to L¹ in formula 1-1-1 described in Scheme 1-1. Formula 1-2-2 means either of formulae AA-H, AB-H and AC-H as described below.

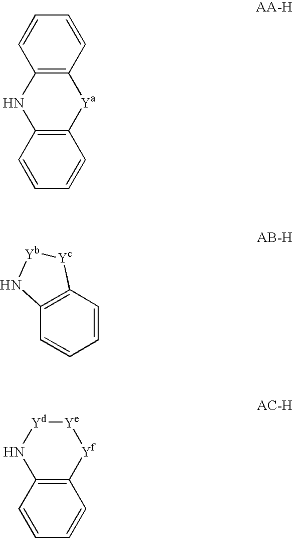

Namely, these compounds are reduced forms of substituent represented by “A” in formula (I) in this specification.

Alkylation of a compound of formula 1-1 with a compound of formula 1-2-1 may be carried out under similar conditions described in Scheme 1-1 in this specification to afford a compound of formula 1-2.

Then, the compound of formula 1-2 thus obtained may be reacted with a compound of formula 1-2-2. A compound of formula 1-2 wherein Z¹ comprises a leaving group may be coupled with a compound of formula 1-2-2 by alkyklation under similar reaction conditions as described in Scheme 1-1 or 1-2 in this specification. A compound of formula 1-2 wherein Z¹ comprises carboxy may be coupled with a compound of formula 1-2-2 by amidation by a peptide formation known to those skilled in the art.

A compound of formula I of the present application wherein A is AB as defined above may be also prepared according to a preparation method described in Scheme 1-3.

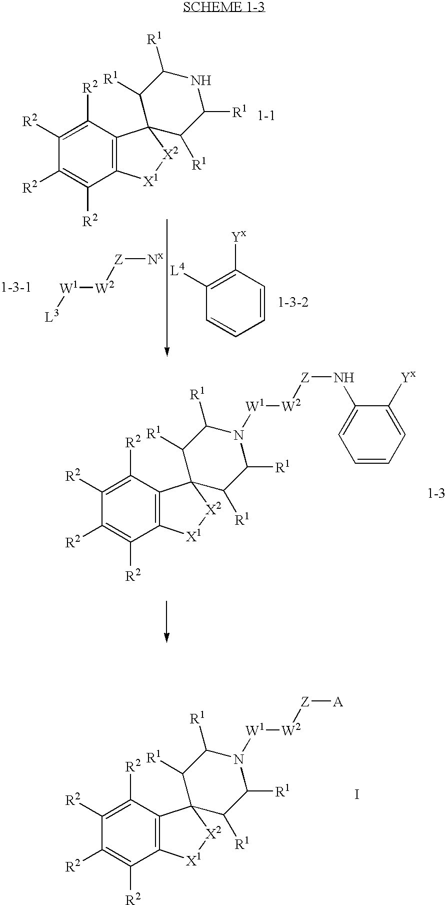

Preparation processes in Scheme 1-3 is preferably useful for compounds of formula I wherein in A is an optionally substituted benzofuzed heteroaryl ring containing a nitrogen atom and additional hetero atoms. A typical benzofuzed ring in the compounds is benzimidazolyl, benzothiazolyl or benzoxazolyl ring.

As shown in Scheme 1-3 the preparation process comprises:
Step 1—reaction between compounds of formula 1-1 may be reacted with compounds of formula 1-3-1, wherein L³ is a leaving group such as halo and N^{x} is amino, phthalnimdo or the like;
Step 2—reaction between compounds obtained in Step 1 with compounds of formula 1-3-2 to give compounds of formula 1-3; and
Step 3—cyclization of compounds of formula 1-3 to yield compounds of formula 1.

The reactions in Step 1 and 2 are alkylations of amine compounds. These reactions may be typically carried out in the presence of potassium ion. Resulting compounds in Step 1 wherein N^{x} is phthalimido may be converted to amine by deprotection with hydrazine prior to Step 2. The reaction in Step 3 may be carried out using carboxylic acids optionally in the presence of acid or a cyano halide.

The subject invention also includes isotopically-labelled compounds, which are identical to those recited in formula (1), but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assay. Tritiated, i.e., ³H, and carbon-14, i.e., ¹⁴C, isotopes are particularly preferred for their ease of presentation and detectability. Further, substitution with heavier isotopes such as deutrium, i.e., ²H, can afford therapeutic advantage resulting from greater metabolic stability, for example increased in Yivo half-life or reduced dosage requirement and, hence, may be preferred in some circumstances. Isotopically labelled compounds of formula (I) of this invention and prodrugs thereof can generally be prepared by carrying out the procedure disclosed in above-disclosed Schemes and/or Examples and Preparations below, by submitting a readily available isotopically labelled reagent for a non-isotopically labelld reagent.

The compounds of Formula (I) of this invention are basic, therefore they will form acid-addition salts. All such salts are within the scope of this invention. However, it is necessary to use an acid addition salt which is pharmaceutically-acceptable for administration to a mammal. The acid-addition salts can be prepared by standard methods. For example, the salts may be prepared by contacting the basic compounds with acid in substantially equivalent proportions in water or an organic solvent such as methanol or ethanol, or a mixture thereof. The salts can be isolated by crystallization from or evaporation of the solvent. Typical salts which can be formed are the hydrochloride, nitrate, sulfate, bisulfate, phosphate, acetate, lactate, citrate, tartrate, succinate, maleate, fumarate, gluconate, saccharate, benzoate, methanesulfonate, ethanesulfonate, benzenesulfonate, p-toluenesulfonate, oxalate and pamoate (1,1′-methylene-bis-(2-hydroxy-3-naphtoate)) salts.

In addition, when the compounds of this invention form hydrates or solvates they are also within the scope of this invention.

The compounds of Formula (I) have been found to possess selective affinity for ORL1-receptors and ORL-1 receptor antagonist activity. Thus, these compounds are useful as an analgesic, anti-inflammatory, diuretic, anesthetic, neuroprotective, anti-hypertensive and anti-anxiety agent, and the like, in mammalian subjects, especially humans in need of such agents. The affinity, antagonist activities and analgesic activity can be demonstrated by the following tests respectively.

Selective Affinity for ORL1-Receptors:

ORL1-Receptor Binding Assay:

The human ORL1 receptor transfected HEK-293 cell membranes were incubated for 45 min at 22° C. with 0.4 nM [³ H]nociceptin, 1.0 mg of wheat germ agglutinin-coated SPA beads and various concentrations of test compounds in a final volume of 200 μl of 50 mM HEPES buffer pH7.4 containing 10 mM MgCl₂ and 1 mM EDTA. Non-specific binding was determined by the addition of 1 μM unlabeled nociceptin. After the reaction, the assay plate was centrifuged at 1,000 rpm for 1 min and then the radioactivity was measured by a Liquid Scintillation Counter.

μ-Receptor Binding Assay:

The human Mu receptor transfected CHO-K1 cell membranes were incubated for 45 min at 22° C. with 1.0 nM [³H]DAMGO, 1.0 mg of wheat germ agglutinin-coated SPA beads and various concentrations of test compounds in a final volume of 200 g 1 of 50 mM Tris-HCl buffer pH7.4 containing 5 mM MgCl₂. Non-specific binding was determined by the addition of 1 μM unlabeled DAMGO. After the reaction, the assay plate was centrifuged at 1,000 rpm for 1 min and then the radioactivity was measured by a Liquid Scintillation Counter.

Each percent non specific binding thus obtained is graphed as a function of compound concentration. A sigmoidal curve is used to determine 50% bindings (i.e., IC₅₀ values).

In this testing, the preferred compounds prepared in the working examples appearing hereafter demonstrated higher binding affinity for ORL1-receptors than for mu-receptors.
IC₅₀(ORL1-receptors)nM/IC₅₀(mu-receptors)nM<1.0
ORL1 Receptor Functional Assay:

The human ORL1 receptor transfected HEK-293 cell membranes were incubated with 400 pM [³⁵S]GTPγS, 50 nM nociceptin and various concentrations of test compounds in assay buffer (20 mM HEPES, 100 mM NaCl, 5 mM MgCl₂, 1 mM EDTA, 5 mM GDP, 1 mM DTT, pH7.4) containing 1.5 mg of wheat germ agglutinin-coated SPA beads for 60 or 90 min at 25° C. in a final volume of 200 μl. Basal binding was assessed in the absence of nociceptin and non-specific binding was defined by the addition of unlabelled 10 mM GTPγS. Membrane-bound radioactivity was detected by at Liquid Scintillation Counter.

Analgesic Tests:

Tail Flick Test in Mice:

The latency time to withdrawal f the tail from radiant heat stimulation is recorded before and after administration of test compounds. Cut-off time is set to 8 sec.

Acetic Acid Writhing Test in Mice:

Acetic acid saline solution of 0.7% (v/v) is injected intraperitoneally (0.16 ml/10 g body weight) to mice. Test compounds are administered before acetic acid injection. As soon as acetic acid injection, animals are placed in a i liter beaker and writhing is recorded for 15 min.

Formalin Licking Test in Mice:

Formalin-induced hind paw licking is initiated by a 20 micro liters subcutaneous injection of a 2% formaline solution into a hind paw of mice. Test compounds are administered prior to formalin injection. Total licking time is recorded for 45 min after formalin injection.

Carrageenan-Induced Mechanical Hyperalgesia Test in Rats:

The response to mechanical nociceptive stimulus is measured using an algesiometer (Ugo Basile, Italy). The pressure is loaded to the paw until rats withdrawal the hind paw. Lambda-Carrageenan saline solution of 1% (w/v) is injected subcutaneously into the hind paw and the withdrawal response is measured before and after the injection. Test compounds are administered at appropriate time point.

Carrageenan-Induced Thermal Hyperalgesia Test in Rats:

The response to thermal nociceptive stimulus is measured using an plantar test apparatus (Ugo Basile, Italy). The radiant heat stimuli is applied to the paw until rats withdrawal the hind paw. Lambda-Carrageenan saline solution of 2% (w/v) is injected subcutaneously into the hind paw and the withdrawal response is measured before and after the injection. This testing method is described in K Hargreaves, et al., Pain 32:77-88, 1988.

Chronic Contriction Injury Model (CCI Model):

Chronic contriction injury is made according to Bennett's method (Bennett, et al., Pain 83:169-182, 1999). Tactile allodynia in rats is assessed using the von Frey hairs (Stoelting, Ill.) before and after administration with test compounds.

The compounds of Formula (I) of this invention can be administered by conventional pharmaceutical practice via either the oral, parenteral or topical routes to mammals, for the treatment of the indicated diseases. For administration to human patient by either route, the dosage is in the range of about 0.01 mg/kg to about 3000 mg/kg body weight of the patient per day, preferably about 0.01 mg/1 g to about 1000 mg/kg body weight per day administered singly or as a divided dose. However, variations will necessarily occur depending upon the weight and condition of the subject being treated, compound employed, the disease state being treated and the particular route of administration chosen.

The compounds of the present invention may be administered alone or in combination with pharmaceutically acceptable carriers by either of the above routes previously indicated, and such administration can be carried out in single or multiple doses. Generally, the compounds can be combined with various pharmaceutically acceptable carriers in the form of tablets, powders, capsules, lozenges, trochees, hard candies, powders, sprays, creams, salves, suppositories, jellies, gels, pastes, lotions, ointments, suspensions, solutions, elixirs, syrups or the like. Such pharmaceutical carriers include solvents, excipients, coating agents, bases, binders, lubricants, disintegrants, solubilizing agents, suspending agents, emulsifing agents, stabilizers, buffering agents, tonicity agents, preservatives, flavorating agents, aromatics, coloring agents and the like.

For example, the tablets can contain various excipients such as starch, lactose, glucose, microcrystalline cellulose, calcium sulfate, calcium carbonate, talc, titanium oxide and the like, coating agents such as gelatin, hydroxypropylcellulose and the like, binding agents such as gelatin, gum arabic, methylcellulose and the like, and the disintegrating agents such as starch, agar, gelatine, sodium hydrogencarbonate and the like. Additionally, lubricating agents such as magnesium stearate and talc are often very useful for tabletting purposes. Solid compositions of a similar type may also be employed as fillers in gelatine capsules; preferred materials in this connection also include lactose as well as high molecular weight polyethylene glycols. When aqueous suspensions and/or elixirs are desired for oral administration, the active ingredient may be combined with various sweetening or flavoring agents, coloring matter or dyes, and, if so desired, emulsifying and/or suspending agents as well, together with diluents such as water, ethanol, propylene glycol, glycerin and various like combinations thereof.

In general, the therapeutically-effective compounds of this invention are present in such oral dosage forms at concentration levels ranging 5% to 70% by weight, preferably 10% to 50% by weight.

The compounds of the present invention in the form of a solution may be injected parenterlly such as intradermaly, subcutaneously, intravenously or intramuscularly. For example the solutions are sterile aqueous solutions, aqueous suspensions and an edible oil solutions. The aqueous solutions may be suitably buffered (preferably pH>8), and may contain enough salts or glucose to make the solution isotonic with blood. The aqueous solutions are suitable for intravenous injection purposes. The aqueous suspensions may contain a suitable dispersing or suspending agents such as sodium carboxymethylcellulose, methylcellulose, polyvinylpyrrolidone or gelatin. The aqueous suspensions can be used for subcutaneous or intramuscular injections. The edible oil such as cottonseed oil, sesame oil, coconut oil or peanut oil can be employed for the edible oil solutions. The oil solutions are suitable for intra-articular, intramuscular and subcutaneous injection. The preparation of all these solutions under sterile conditions is readily accomplished by standard pharmaceutical techniques well-known to those skilled in the art.

It is also possible to administer the compounds of the present invention topically when treating inflammatory conditions of the skin and this may preferably be done by way of creams, jellies, gels, pastes, ointments and the like, in accordance with standard pharmaceutical practice.

### EXAMPLES AND PREPARATIONS

The present invention is illustrated by the following examples and preparation. However, it should be understood that the invention is not limited to the specific details of these examples and preparations. Melting points were taken with a Buchi micro melting point apparatus and is not corrected. Infrared Ray absorption spectra (IR) were measured by a Shimadzu infrared spectrometer (IR-470). ¹H and ¹³C nuclear magnetic resonance spectra (NMR) were measured in CDCl₃ by a JEOL NMR spectrometer (JNM-GX270, 270 MHz) unless otherwise indicated and peak positions are expressed in parts per million (ppm) downfield from tetramethylsilane. The peak shapes are denoted as follows: s, singlet; d, doublet; t, triplet; m, multiplet; br, broad.

Analytical data of compounds, which can be prepared according to General Procedures A and B or were prepared in Examples hereinafter disclosed, can be taken by utilizing Waters LC-MS system (LC as 2690, ZMD as MS).

Analytical condition for LC-MS: Column YMC CombiScreen basic 4.6 mm×50 mm, Flow rate 1 mL/min.; Mobile phase 20% MeOH/80% 0.1%/HCO₂H in H₂O programmed over 5 min to 90% MeOH/10% 0.1% HCO₂H in H₂O. Hold for 5 min.; Wave length 220400 nm. MS detector ApcI Cone 30 Volts.

### Preparation 1

### 2,3-Dihydro-1′-[2-(ethoxycarbonyl)ethyl]spiro[1H-indene-1,4′-piperidine]

A mixture of 2,3-dihydrospiro[1H-indene-1,4′-piperidine]hydrochloride (1.00 g, 4.47 mmol, this was prepared according to known procedure:M. S. Chambers et al, J. Med. Chem. 1992, 35, 2033), ethyl 3-bromopropionate (1.62 g, 8.94 mmol) and NjV-diisopropylethylamine (1.73 g, 13.4 mmol) in EtOH (20 ml) was stirred at 65° C. for 18 h. Then the reaction mixture was concentrated, basified with NaHCO3 solution, and extracted with CH₂Cl₂. The extracts combined were dried (MgSO4), filtered, and concentrated. The residue was purified by silica gel column chromatography (CH₂Cl₂/MeOH: 40/1 as eluent) to give 1.28 g (99%) of title compound as colorless oil.

¹H NMR (300 MHz, CDCl₃) δ 7.22-7.12 (4H, m), 4.46 (2H, q, J=7.2 Hz), 2.95-2.83(6H, m), 2.80-2.73 (2H, m), 2.60-2.52(2H, m), 2.28-2.18 (2H, m), 2.03-1.87 (4H, m), 1.60-1.50 (2H, m), 1.28 (3H, t, J=7.2 Hz).

MS(EI direct) m/z: 287(M)⁺.

### Preparation 2

### 2,3-Dihydro-1′-[2-(carboxy)ethyl]spiro[1H-indene-1,4′-piperidine]hydrochloride

A mixture of 2,3-dihydro-1′-[2-(ethoxycarbonyl)ethyl]spiro[1H-indene-1,4′-piperidine] (1.28 g, 4.45 mmol), 2N HCl (10 ml) and AcOH (10 ml) was stirred at 100° C. for 20h. After cooling down to 0° C., the resulting white solid appeared was collected by filtration, washed with AcOEt, and dried to afford 1.13 g (86%) of title compound as a white solid.

¹H NMR (300 MHz, DMSO-d6) δ 10.20 (1H, br.s), 7.25-7.10 (4H, m), 3.50-3.00 (6H, m), 2.89-2.82 (4H, m), 2.23-2.08 (2H, m), 2.04 (2H, t, J=7.2 Hz), 1.70-1.60 (2H, m). MS(ESI positive) m/z: 260(M+H)⁺.

### Preparation 3

### 2,3-Dihydro-1′-[2-(chloroformyl)ethyl]spiro[1H-indene-1,4′-piperidine]hydrochloride

To a stirred suspension of 2,3-dihydro-1′-[2-(carboxy)ethyl]spiro[1H-indene-1,4′-piperidine]hydrochloride (0.80 g, 2.70 mmol) in thionyl chloride (6 ml) was added DMF (0.2 ml) at room temperature. After 1 h stirring, the reaction mixture was diluted with mixed solvents (CH₂Cl₂/hexane: 1/1). The resulting solid appeared was collected by filtration and dried to give 0.77 g (91%) of title compound as white solid.

¹H NMR (300 MHz, DMSO-d6) δ 10.81 (1H, br.s), 7.25-7.09 (4H, m), 3.52-3.42 (2H, m), 3.36-3.27 (2H, m), 3.17-3.01 (2H, m), 2.94-2.86 (4H, m), 2.31-2.18 (2H, m), 2.06 (2H, t, J=7.2 Hz), 1.69-1.59 (2H, m). MS(EI direct) m/z: 277(M)⁺.

### Example 1

### 2,3-Dihydro-1′-[3-(2-methoxycarbonylindolin-1-yl)-3-oxopropyl]spiro[1H-indene-1,4′-piperidine]hydrochloride

To a stirred solution of methyl indoline-2-carboxylate (152 mg, 0.86 mmol) and triethylamine (0.36 ml, 2.58 mmol) in CH₂Cl₂ (5 ml) was added 2,3-dihydro-1′-[2-(chloroformyl)ethyl]spiro[1H-indene-1,4-piperidine]hydrochloride (270 mg, 0.86 mmol) at room temperature and the resulting reaction mixture was stirred for 5 h. The reaction mixture was poured into a saturated aqueous NaHCO3 solution and extracted with CH2Cl2. The extracts combined were washed with brine, dried (MgSO4), filtered, and concentrated. The residue was purified by silica gel column chromatography (CH2Cl2/MeOH: 30/1 as an eluent) to give 160 mg (44%) of title product as colorless amorphous solid.

¹H NMR (270 MHz, CDCl3) δ 8.28-8.19 (0.5H, m), 7.26-7.10 (6.5H, m), 7.07-7.00 (1H, m), 5.25-5.00(1H, m), 3.77 (3H, br.s), 3.70-3.40 (1H, m), 3.35-2.80 (8H, m), 2.75-2.50 (1H, m), 2.37-2.20 (2H, m), 2.07-1.40 (41, m), 1.62-1.50 (2H, m). 33 mg of this solid was dissolved in HCl solution in MeOH (1 ml), concentrated, solidified with CH2Cl2/hexane, washed with ether, and collected by filtration to give 29 mg of title compound as white amorphous solid.

¹H NMR (270 MHz, CDCl3) δ 12.40 (1H, br.s), 8.18 (0.75H, d, J=8.2 Hz), 7.43-7.30 (1.25H, m), 7.26-7.15 (5H, m), 7.07 (1H, t, J=7.2 Hz), 5.25-5.10 (1H, m), 3.85 (2.25H, s), 3.74 (0.75H, s), 3.72-3.32 (6H, m), 3.20-2.60 (6H, m), 2.07 (2H, t, J=7.1 Hz), 1.80-1.50 (4H, m).

MS (ESI positive) m/z: 419 (M+H)⁺.

IR(KBr): 3310, 2934, 2561, 1744, 1655, 1481, 1418, 1207, 758 cm⁻¹

Anal. Calcd for C26H30N2O3-HCl-0.8H2O:C, 66.53; H, 7.00; N, 5.97. Found: C, 66.55; H, 7.00; N, 5.97.

### Preparation 4

### 2,3-Dihydro-1′-[2-(2-hydroxyethoxycarbonyl)ethyl]spiro[1H-indene-1,4′-piperidine]

A mixture of 2,3-dihydrospiro[1H-indene-1,4′-piperidine]hydrochloride (0.31 g, 1.39 mmol, this was prepared according to known procedure:M. S. Chambers et al, J. Med. Chem. 1992, 35, 2033), ethyl 3-bromopropionate (0.50 g, 2.77 mmol) and N,N-diisopropylethylamine (0.54 g, 4.17 mmol) in ethylene glycol (10 ml) was stirred at 80° C. for 16 h. Then the reaction mixture was poured into a saturated aqueous NaHCO3 solution, and extracted with AcOEt. The extracts combined were dried (MgSO4), filtered, and concentrated. The residue was purified by silica gel column chromatography (CH2Cl2/MeOH: 20/1 as an eluent) to give 0.37 g (88%) of title compound as colorless oil.

¹H NMR (300 MHz, CDCl3) δ 7.25-7.15 (4H, m), 4.37-4.33 (2H, m), 3.84-3.78 (2H, m), 3.01-2.94 (2H, m), 2.94 (2H, t, J=8.1 Hz), 2.78-2.72 (211, m), 2.64-2.58 (21, m), 2.14-2.05 (2H, m), 2.04-1.91 (4H, m, including 211, t, J=8.1 Hz at 2.00 ppm), 1.60-1.50 (8H, m). MS(EI direct) m/z: 303(M)⁺.

### Preparation 5

### 2,3-Dihydro-1′-12-(carboxy)ethyl]spiro[1H-indene-1,4′-piperidine]

A mixture of 2,3-dihydro-1′-[2-(2-hydroxyethoxycarbonyl)ethyl]spiro[1H-indene-1,4′-piperidine] (0.37 g, 1.22 mmol), 2N NaOH (4 ml) and EtOH (10 ml) was refluxed with stirring for 16 h. After cooling down to 0° C., the resulting mixture was neutralized with a 2N HCl solution and extracted with CH2Cl2 and AcOEt. The extracts combined were dried (MgSO4), filtered, and concentrated to give 120 mg (38%) of title compound as an yellow solid.

¹H NMR (270 MHz, CDCl3) δ 7.26-7.20 (4H, m), 3.52-3.43 (2H, m), 3.25-3.15 (2H, m), 2.96 (2H, t, J=8.1 Hz), 2.91-2.81 (2H, m), 2.70-2.63 (2H, m), 2.33-2.19 (2H, m), 2.08 (2H, t, J=8.1 Hz), 1.81-1.70 (2H, m).

### Example 2

### 2,3-Dihydro-1′-[3-(indolin-1-yl)-3-oxopropyl]spiro[1H-indene-1,4′-piperidine]hydrochloride

A mixture of 2,3-dihydro-1′-[2-(carboxy)ethyl]spiro[1H-indene-1,4′-piperidine] (14 mg, 0.054 mmol), indoline (12 ll, 0.108 mmol), WSC (21 mg, 0.108 mmol), HOBt (15 mg, 0.108 mmol), and triethylamine (23 μl, 0.162 mmol) in CH2Cl2 (3 ml) was stirred at room temperature overnight. A saturated aqueous NaHCO3 solution was added to the reaction mixture and aqueous layer was removed by decantation. The separated organic layer was dried (MgSO4), filtered, and concentrated. The resulting residue was purified by preparative TLC (1 mm thick silica gel plate: CH2Cl2/MeOH:10/1) to afford 12 mg (62%) of colorless oil.

¹H NMR (270 MHz, CDCl3) δ 8.24 (1H, d, J=8.1 Hz), 7.24-7.12 (6H, m), 7.05-6.98 (1H, m), 4.10 (2H, t, J=8.4 Hz), 3.21 (2H, t, J=8.4 Hz), 3.00-2.86 (6H, m), 2.7&2.68 (2H, m), 2.36-2.24 (2H, m), 2.03 (2H, t, J=7.2 Hz), 2.03-1.90 (2H, m), 1.63-1.53 (2H, m). This was converted to HCl salt similar to that described in Example 1 to afford 12 mg of title compound as white solid. MS (ESI positive) m/z: 361 (M+H)⁺.

### Example 3

2,3-Dihydro-1′-[3-(benzimidazol-2-one-1-yl)propyl]spiro[1H-indene-1,4′-piperidine]formate

In a one-dram vial were mixed a solution of 1-(3-bromopropyl)benzimidazol-2-one (38 mg, 0.15 mmol, this was reported in EP181793) in ethyleneglycol (1 ml) and a solution of 2,3-dihydrospiro[1H-indene-1,4′-piperidine]hydrochloride (11 mg, 0.05 mmol) and N,N-diisopropylethylamine (17 μl, 0.1 mmol) in ethyleneglycol (1 ml), and the mixture was agitated by shaking at 100° C. After 24 h, the reaction mixture was loaded onto a BondElute® SCX cartridge (500 mg/3 ml) which was preconditioned with MeOH (1 ml). The solid-phase matrix was washed with MeOH (5 ml) and then eluted with 2M ammonia/MeOH solution (2 ml). The eluate was concentrated under reduced pressure to give an oil, to which were added CH2Cl2 (1 ml) and PS-NCO (1.3 mmol/g; 75 mg, 0.1 mmol). The resulting suspension was shaken at room temperature for 2 h. Insoluble polymers were removed by filtration, and the filtrate was concentrated to dryness by vacuum centrifuge to give an amorphous solid, which was purified with reverse-phase preparatory HPLC (0.1% HCO2H-MeOH) to give the title compound as a formic acid salt (6.2 mg; 27% yield).

ESI-MS (LC/MS): Calcd. for C₂₃H₂₇N₃O: [M+H]⁺=362.22. Found: 362.58

HPLC purity: 97.8% (UV 210-400 nm); retention time: 3.58 min

### Preparation 6

### 2,3-Dihydro-1′-(3-hydroxypropyl)spiro[1H-indene-1,4′-piperidine]

A mixture of 2,3-dihydrospiro[1H-indene-1,4′-piperidine]hydrochloride (0.5 g, 2.23 mmol, this was prepared according to known procedure:M. S. Chambers et al, J. Med. Chem. 1992, 35, 2033), 3-bromopropanol (0.3 ml, 3.35 mmol), K2CO3 (924.6 mg, 6.69 mmol), and KI (185.9 mg, 1.12 mmol) in MeCN (30 ml) was refluxed with stirring for 18 h. After cooling down to room temperatute, water (30 ml) was added to the reaction mixture and extracted with CH2Cl2 (20 ml×3). The extracts combined were dried (Na2SO4), filtered, and concentrated to give 574.7 mg of crude product. This was purified by silica gel column chromatography (CH2Cl2/MeOH: 15/1 as an eluent) to afford 288.7 mg (53%) of title compound as pale yellow white solid.

¹H NMR (270 MHz, CDCl3) δ 7.26-7.12 (4H, m), 3.86 (2H, t, J=5.3 Hz), 3.34-3.24 (2H, m), 2.95-2.88 (4H, m), 2.56-2.42 (2H, m), 2.26-2.10 (2H, m), 2.03 (2H, t, J=7.3 Hz), 1.96-1.85 (2H, m), 1.71-1.60 (2H, m).

MS(EI direct) m/z: 245(M)⁺.

### Preparation 7

### 2,3-Dihydro-1′-(3-mesyloxypropyl)spiro[1H-indene-1,4′-piperidine]

To a stirred solution of 2,3-dihydro-1′-(3-hydroxypropyl)spiro[1H-indene-1,4′-piperidine] (288.7 mg, 1.18 mmol) in CH2Cl2 (10 ml) was added triethylamine (0.3 ml, 2.12 mmol) followed by dropwise addition of mesyl chloride (0.11 ml, 1.42 mmol) at 0° C. After 1 h stirring at 0° C., the reaction mixture was poured into a saturated aqueous NaHCO3 solution and extracted with CH2Cl2 (30 ml×3). The extracts combined were washed with brine, dried (Na2SO4), filtered, and concentrated to give 330.4 mg of title compound as yellow oil, which was used for the next reaction without purification.

¹H NMR (270 MHz, CDCl3) δ 7.26-7.11 (4H, m), 4.34 (2H, t, J=6.4 Hz), 3.03 (3H, s), 2.96-2.80 (4H, m), 2.51 (2H, t, J=7.2 Hz), 2.24-2.12 (2H, m), 2.05-1.84 (6H, m), 1.62-1.50 (2H, m).

MS(EI direct) m/z: 323(M)⁺.

### Example 4

### 2,3-Dihydro-1′-[3-(benzothiazol-2-one-1-yl)propyl]spiro[1H-indene-1,4′-piperidine]hydrochloride

To a stirred solution of NaH (13.6 mg, 0.34 mmol, 60% oil dispersion in mineral oil, which was removed by washing with n-hexane (2 ml×2) before use) and benzothiazol-2-one (46.9 mg, 0.31 mmol) in DMF (1 ml) was added a solution of 2,3-dihydro-1′-(3-mesyloxypropyl)spiro[1H-indene-1,4′-piperidine] (50 mg, 0.155 mmol) in DMF (1.5 ml) at 0° C. The reaction mixture was heated to 100° C. with stirring for 21 h. The reaction mixture was cooled to 0° C. and NaHCO3 solution was added to the reaction mixture, then extracted with CH2Cl2 (15 ml×3). The extracts combined were washed with brine, dried (Na2SO4), and filtered. The filtrate was evaporated in vacuo to afford 87 mg of crude product, which was purified by preparative TLC (1 mm thick silica gel plate: CH2Cl2/MeOH:20/1, 2 times developed) to give the product. It was purified again by preparative TLC (1 mm thick silica gel plate: n-hexane/AcOEt:2/1, 2 times developed) to give 36.4 mg (62%) of free form of the title compound as pale yellow oil.

¹H NMR (270 MHz, CDCl3) δ 7.45-7.41 (1H, m), 7.35-7.28 (1H, m), 7.24-7.12 (6H, m), 4.05 (2H, t, J=6.9 Hz), 2.92-2.80 (4H, m), 2.46 (2H, t, 3=6.9 Hz), 2.19-2.08 (2H, m), 2.04-1.83 (6H, m), 1.58-1.48 (2H, m).

MS (ESI positive) m/z: 379 (M+H)⁺.

This was converted to HCl salt similar to that described in Example 1 to give 24.7 mg of HCl salt as white solid.

IR(KBr): 3416, 2939, 2500, 1678, 1474, 748 cm⁻¹

Anal. Calcd for C23H26N2OS—HCl-0.4H2O: C, 65.43; H, 6.64; N, 6.63. Found: C, 65.66; H, 6.81; N, 6.36.

### Preparation 8

### 2,3-Dihydro-1′-[3-(2-carboxyindolin-1-yl)-3-oxopropyl]spiro[1H-indene-1,4′-piperidine]

A mixture of 2,3-dihydro-1′-[3-(2-methoxycarbonylindolin-1-yl)-3-oxopropyl]spiro[1H-indene-1,4′-piperidine] (42 mg, 0.092 mmol, this was prepared in Example 1) and 2N HCl (1 ml) in acetic acid (3 ml) was heated at 90° C. with stirring for 16 h. The reaction mixture was concentrated to give solid which was triturated in AcOEt. The solid was collected by filtration to afford 30 mg as a pale red solid. This showed no methyl singlet peak of methyl ester in starting material in 1H NMR spectroscopy. This was used for the next reaction without purification.

### Example 5

### 2,3-Dihydro-1′-{3-[2-(N-methylaminocarbonyl)indolin-1-yl]-3-oxopropyl}spiro[1H-indene-1,4′-piperidine]hydrochloride

A mixture of 2,3-dihydro-1′-[3-(2-carboxyindolin-1-yl)-3-oxopropyl]spiro[1H-indene-1,4′-piperidine] (30 mg, 0.068 mmol), methylamine hydrochloride (10 mg, 0.136 mmol), WSC (26 mg, 0.136 mmol), HOBt (19 mg, 0.136 mmol), and triethylamine (47 μl, 0.34 mmol) in CH2Cl2 (4 ml) was stirred at room temperature for 16 h. The reaction mixture was poured into saturated aqueous NaHCO3 solution, extracted with CH2Cl2, dried (MgSO4), filtered, and concentrated. The residue was purified by preparative TLC (1 mm thick silica gel plate, CH2Cl2/MeOH: 10/1) to afford 6 mg (21%) of free form of the title compound as white solid.

¹H NMR (270 MHz, CDCl3) δ 8.20 (1H, br.s), 7.26-7.00 (7H, m), 6.40 (11, br.s), 5.30-4.90 (1H, m), 3.75-3.20 (2H, m), 3.10-2.90 (4H, m), 2.90 (2H, t, J=7.4 Hz), 2.79 (3H, d, 3=4.8 Hz), 2.45-2.25 (4H, m), 2.02 (2H, t, J=7.4 Hz), 2.09-1.90 (2H, m), 1.63-1.53 (2H, m).

MS (ESI positive) m/z: 418 (M+H)⁺.

This was converted to HCl salt similar to that described in Example 1 to give 6 mg of HCl salt as a pale gray solid.

MS (ESI positive) m/z: 418 (M+H)⁺.

### Example 6

### 2,3-Dihydro-1′-[2-(1,1-dioxido-3-oxo-1,2-benzisotiazol-2(3R)-yl)ethyl]spiro[1H-indene-1,4′-piperidine]

A mixture of 2,3-dihydrospiro[1H-indene-1,4′-piperidine]hydrochloride (80 mg, 0.357 mmol), N-2-(mesyloxy)ethylsaccharin (130.7 mg, 0.428 mmol), K2CO3 (148 mg, 1.07 mmol) and KI (29.7 mg, 0.179 mmol) in MeCN (6 ml) was refluxed with stirring for 18 h. After cooling down to room temperature, the reaction mixture was poured into aqueous NaHCO3 solution and extracted with CH2Cl2 (20 ml×3). The extracts combined were washed with brine, dried (Na2SO4), filtered, and concentrated to give 191.7 mg of crude product, which was purified by preparative TLC (1 mm thick silica gel plate, CH2Cl2/MeOH: 25/1). Then extracted product was purified again by preparative TLC (n-hexane/AcOEt:1/1, 2 times developed) to give 31.6 mg (22%) of title compound as pale yellow oil.

¹H NMR (270 MHz, CDCl3) δ 8.10-8.05 (1H, m), 7.96-7.80 (3H, m), 7.24-7.12 (4H, m), 3.96 (2H, dd, J=7.2, 7.6 Hz), 3.04-2.95 (2H, m), 2.89 (2H, t, J=7.4 Hz), 2.85 (2H, t, J=7.6 Hz), 2.41-2.28 (2H, m), 2.06-1.88 (4H, m), 1.96-1.88 (2H, m).

MS (ESI positive) m/z: 397 (M+H)⁺.

IR(r): 2924, 1734, 1327, 1180, 752 cm⁻¹

Anal. Calcd for C22H24N2O3S-0.2H2O: C, 66.04; H, 6.15, N, 7.00. Found: C, 66.06; H, 6.27; N, 6.73.

### Example 7

### 2,3-Dihydro-1′-[3-(2-oxo-3,4-dihydro-[(2B)quinolinyl)propyl]spiro[1H-indene-1,4′-piperidine]citrate

This was prepared according to the procedure described in Example 4 using 3,4-dihydro-2(1H)-quinolinone instead of benzothiazol-2-one. Yield was 38.1 mg (66%). Product was pale yellow oil.

¹H NMR (270 MHz, CDCl3) δ 7.28-7.10 (7H, m), 6.99 (1H, ddd, 3=1.2, 7.2, 7.4 Hz), 4.02 (2H, dd, J=7.3, 7.6 Hz), 2.95-2.84 (6H, m), 2.68-2.61 (2H, m), 2.52-2.45 (2H, m), 2.26-2.12 (2H, m), 2.03-1.84 (6H, m), 1.60-1.50 (2H, m).

To a stirred solution of this oil (36.3 mg, 0.097 mmol) in MeOH (1.5 ml) was added citric acid (18.6 mg, 0.097 mmol) at room temperature. After 2 h stirring, the solvent was evaporated to give 45 mg of citric acid salt as white amorphous solid.

MS (ESI positive) m/z: 375 (M+H)⁺.

IR(KBr): 3402, 2945, 2600, 1728, 1657, 1601, 1387, 1190, 758 cm⁻¹

Anal. Calcd for C25H30N20—C6H8O7-H2O: C, 63.68; H, 6.90; N, 4.79. Found: C, 63.90; H, 6.86; N, 4.63.

### Example 8

### 2,3-Dihydro-1′-[3-(3-methyl-2-oxo-3,4dihydro-1 (2H)-quinazolinyl)propyl]spiro[1H-indene-1,4′-piperidine]citrate

This was prepared according to the procedure described in Example 4 using 3,4-dihydro-3-methyl-2(1H)-quinazolinone instead of benzothiazol-2-one. Yield was 28 mg (46%). Product was pale yellow oil.

¹H NMR (270 MHz, CDCl3) δ 7.28-7.10 (5H, m), 7.08-6.91 (3H, m), 4.37 (2H, s), 3.94 (2H, dd, J=7.4, 7.6 Hz), 3.02 (3H, s), 3.01-2.86 (4H, m), 2.58-2.50 (2H, m), 2.29-2.16 (2H, m), 2.06-1.88 (6H, m), 1.62-1.50 (2H, m).

To a stirred solution of this oil (28 mg, 0.072 mmol) in MeOH (1.5 ml) was added citric acid (13.8 mg, 0.072 mmol) at room temperature. After 1 h stirring, the solvent was evaporated to give 36.8 mg of citric acid salt as white amorphous solid.

MS (ESI positive) m/z: 390 (M+H)⁺.

IR(KBr): 3416, 2939, 2600, 1728, 1657, 1641, 1605, 1489, 1213,758 cm⁻¹

Anal. Calcd for C25H31N30—C6H8O7-H2O: C, 62.09; H. 6.89; N, 7.01. Found: C, 62.26; H, 6.88; N, 6.75.

### Example 9

### 2,3-Dihydro-1′-[3-(2-oxo-1,3-benzoxazol-3(21H)-yl)propyl]spiro[1H-indene-1,4′-piperidine]citrate

This was prepared according to the procedure described in Example 4 using benzoxazol-2-one instead of benzothiazol-2-one. Yield was 29.4 mg (52%). Product was reddish brown oil.

¹H NMR (300 MFz, CDCl3) δ 7.26-7.06 (8H, m), 3.94 (2H, t, J=6.8 Hz), 2.88 (2H, t, J=7.3 Hz), 2.45 (2H, t, J=6.8 Hz), 2.16-2.06 (2H, m), 2.05-1.94 (4H, m), 1.90-1.78 (2H, m), 1.55-1.47 (2H, m).

To a stirred solution of this oil (29.4 mg, 0.081 mmol) in MeOH (1.5 ml) was added citric acid (15.6 mg, 0.081 mmol) at room temperature. After 1 h stirring, the solvent was evaporated to give 32.5 mg of citric acid salt as red amorphous solid.

MS (ESI positive) m/z: 363 (M+H)⁺.

IR(KBr): 3437, 2939, 2544, 1771, 1732, 1589, 1487, 1371, 1254, 756 cm⁻¹

Anal. Calcd for C23H26N2O2-C6H8O7-0.5H2O: C, 61.80; H, 6.26; N, 4.97. Found: C, 61.41; H, 6.24; N, 4.88.

### Example 10

### 2,3-Dihydro-1′-[3-(2-carboxyindolin-1-yl)-3-oxopropyl]spiro[1H-indene-1,4′-piperidine]

To a stirred solution of 2,3-dihydro-1′-[3-(2-methoxycarbonylindolin-1-yl)-3-oxopropyl]spiro[1H-indene-1,4′-piperidine] (125 mg, 0.3 mmol, this was prepared in Example 1) in THF (3 ml) and MeOH (1 ml) was added 2N NaOH (0.6 ml, 1.2 mmol) at room temperature. After 16 h stirring at room temperature, the reaction mixture was neutralized with 2N HCl (0.6 ml) and 4 drops of saturated aqueous NaHCO3 solution, diluted with water (5 ml), and extracted with CH2Cl2. The extracts combined were dried (MgSO4), filtered, and concentrated to give 105 mg (87%) of title product as white solid.

¹H NMR (270 MHz, DMSO-d6) δ 8.09 (1H, L, J=8.4 Hz), 7.30-6.80 (8H, m), 5.35-5.15 (1H, m), 3.70-2.75 (12H, m), 2.10-1.95 (4H, m), 1.70-1.55 (2H, m).

MS (ESI positive) m/z: 405 (M+H)⁺.

### Example 11

### 2,3-Dihydro-1′-[3-(2-N,N-dimethylaminocarbonylindolin-1-yl)-3-oxopropyl]spiro[1H-indene-1,4′-piperidine]hydrochloride

A mixture of 2,3-dihydro-1′-[3-(2-carboxyindolin-1-yl)-3-oxopropyl]spiro[1H-indene-1,4′-piperidine] (23 mg, 0.057 mmol, this was prepared in Example 10), dimethylamine hydrochloride (14 mg, 0.17 mmol), WSC (22 mg, 0.114 mmol), HOBt (16 mg, 0.114 mmol), and triethylamine (40 L, 0.29 mmol) in CH2Cl2 (3 ml) was stirred at room temperature for 20 h. The reaction mixture was diluted with saturated aqueous NaHCO3 solution and extracted with CH2Cl2. The extracts combined were dried (MgSO4), filtered, and concentrated. The residue was purified by preparative TLC (1 mm thick plate, CH2Cl2/MeOH: 10/1) to give 20 mg (81%) of free form of title product as colorless oil.

¹H NMR (270 MHz, CDCl3) δ 8.29 (0.5H, d, J=7.9 Hz), 7.65-6.95 (7.5H, m), 5.50-5.40 (0.5H, m), 5.35-5.25 (0.5H, m), 3.77-3.60 (0.5H, m), 3.53-3.35 (0.5H, m), 3.22-2.20 (17H, m, including 1.5H, s at 3.19 ppm, 1.5H, s at 3.16 ppm, 1.5H, s at 3.01 ppm, 1.5H, s at 2.98 ppm, 2H, t, J=7.4 Hz at 2.90 ppm), 2.15-1.90 (4H, m, including 2H, t, J=7.4 Hz at 2.02 ppm), 1.75-1.50 (2H, m).

This was converted to HCl salt similar to that described in Example 1 to give 15 mg of HCl salt as a white solid.

¹H NMR (270 MHz, CDCl3) δ 12.13 (1H, br.s), 8.25 (1H, d, J=8.2 Hz), 7.40-7.00 (7H, m), 5.65-5.50 (1H, m), 3.85-2.50 (18H, m including 3H, s at 3.28 ppm, 3H, s at 3.05 ppm, and 2H, t, J=7.4 Hz at 2.95 ppm), 2.04 (2H, t, J=7.4 Hz), 1.80-1.50 (4H, m).

MS (ESI positive) m/z: 432 (M+H)⁺.

IR(KBr): 3446, 2936, 2561, 1653, 1483, 1458, 1398, 1271, 758 cm⁻¹

Anal. Calcd for C27H33N3O2-HCl-H2O: C, 66.72; H, 7.47; N, 8.65. Found: C, 66.48; H, 7.48; N, 8.56.

### Example 12

### 2,3-Dihydro-1′-[3-(2-morpholinocarbonylindolin-1-yl)-3-oxopropyl]spiro[1H-indene-1,4′-piperidine]hydrochloride

This was prepared according to the procedure described in Example 11 using morpholine instead of dimethylamine hydrochloride. 23 mg (86%) of free form of title compound was obtained as colorless oil.

¹H NMR (270 MD, CDCl3) δ 8.35-8.23 (0.4H, m), 7.33-7.05 (6.6H, m), 7.01 (1H, br.dd, J=7.4, 8.4 Hz), 5.50-5.40 (0.6H, m), 5.37-5.25 (0.4H, m), 3.90-3.35 (9H, m), 3.13-2.20 (11H, m, including 2H, t, J=7.5 Hz at 2.90 ppm), 2.10-1.90 (4H, m, including 2H, t, J=7.4 Hz at 2.02 ppm), 1.65-1.50 (2H, m).

This was converted to HCl salt similar to that described in Example 1 to give 18 mg of HCl salt as a white solid.

¹H NMR (270 MHz, CDCl3) δ 8.25 (1H, d, J=7.9 Hz), 7.40-7.00 (8H, m), 5.80-5.70 (1H, m), 4.08-3.35 (13H, m), 3.13-2.50 (7H, m, including 2H, t, J=7.4 Hz at 2.95 ppm), 2.04 (2H, t, J=7.6 Hz), 1.80-1.50 (4H, m).

MS (ESI positive) m/z: 474 (M+H)⁺.

IR(KBr): 2928, 2550, 1655, 1119, 752 cm⁻¹

Anal. Calcd for C29H35N3O3-HCl-0.7H2O: C, 66.64; H, 7.21; N, 8.04. Found: C, 66.85; H, 7.32; N, 7.89.

### Example 13

### 2,3-Dihydro-1′-[3-[2-(aminocarbonyl)-2,3-dihydro-1H-indol-1-yl]-3-oxopropyl]spiro[1H-indene-1,4′-piperidine]hydrochloride

To a stirred suspension of 2,3-dihydro-1′-[3-(2-carboxyindolin-1-yl)-3-oxopropyl]spiro[1H-indene-1,4′-piperidine] (20 mg, 0.049 mmol, this was prepared in Example 10) in MeCN (4 ml) was added 1,1′-carbonyldiimidazole (9 mg, 0.054 mmol) at room temperature and resulting mixture was refluxed for 0.5 h. Triethylamine (10 μl) was added to the reaction mixture and reflux was continued for 2 h. To a reaction mixture was added 25% NH4OH (2 ml) and reflux was continued for 2 h. Then the reaction mixture was concentrated, diluted with saturated aqueous NaHCO3 solution, and extracted with CH2Cl2. The extracts combined were dried (MgSO₄), filtered, and concentrated. The residue was purified by preparative TLC (1 mm thick plate, CH2Cl2/MeOH: 10/1) to afford 9 mg (45%) of free form of title compound as colorless amorphous solid.

This compound showed broadened spectra in proton NMR

This was converted to HCl salt similar to that described in Example 1 to give 8 mg of HCl salt as a white solid.

¹H NMR (270 MHz, CDCl3+CD₃OD) δ 8.17 (1H, d, I=7.6 Hz), 7.38-7.03 (8H, m), 5.35-5.10 (1H, m), 3.85-3.20 (10H, m), 3.15-2.35 (6H, m, including 2H, t, J=7.3 Hz at 3.00 ppm), 2.10 (2H, t, J=7.3 Hz), 1.83-1.70 (2H, m).

MS (ESI positive) m/z: 404 (M+H)⁺.

### Example 14

### 2,3-Dihydro-1′-[3-(2-(S)-methoxycarbonylindolin-1-yl)-3-oxopropyl]spiro[1H-indene-1,4′-piperidine]hydrochloride

To a stirred suspension of (2S)-methyl indoline-2-carboxylate hydrochloride (520 mg, 2.43 mmol) in CH2Cl2 (10 ml) was added triethylamine (1.13 ml, 8.1 mmol) at 0° C. After 10 minutes stirring, 2,3-dihydro-1′-[2-(chloroformyl)ethyl]spiro[1H-indene-1,4′-piperidine]hydrochloride (510 mg, 1.62 mmol) was added to the reaction mixture at 0° C. and the resulting reaction mixture was stirred at 0° C. for 4 h. The reaction mixture was quenched with a saturated aqueous NaHCO3 solution and extracted with CH2Cl2. The extracts combined were washed with brine, dried (MgSO4), filtered, and concentrated. The residue was purified by silica gel column chromatography (CH2Cl2/MeOH: 20/1 as an eluent) to give 345 mg (49%) of colorless amorphous solid.

¹H NMR (270 MHz, CDCl3) δ 8.30-8.15 (0.5H, m), 7.35-7.07 (6.5H, m), 7.05-6.95 (1H, m), 5.25-4.98(1H, m), 3.74 (3H, br.s), 3.70-3.35 (1H, m), 3.35-2.45 (9H, m), 2.35-2.15 (2H, m), 2.05-1.85 (4H, m), 1.65-1.48 (2H, m).

24 mg of this solid was dissolved in HCl solution in MeOH (0.5 ml), concentrated, solidified with ether, and collected by filtration to give 22 mg of title compound as white amorphous solid.

MS (ESI positive) m/z: 419 (M+H)⁺.

IR(KBr): 3420, 2951, 2563, 1744, 1661, 1481, 1418, 1207, 758 cm⁻¹

Anal. Calcd for C26H30N2O3-HCl-0.6H2O: C, 67.04; H, 6.97; N, 6.01. Found: C, 67.07; H, 7.10; N, 5.78.

### Example 15

### 2,3-Dihydro-1′-{3-[2-(1-ethylpyrrolydin-3-yl)aminocarbonylindolin-1-yl]-3-oxopropyl}spiro[1H-indene-1,4′-piperidine]dihydrochloride

A mixture of 2,3-dihydro-1′-[3-(2-carboxyindolin-1-yl)-3-oxopropyl]spiro[1H-indene-1,4′-piperidine] (35 mg, 0.087 mmol, this was prepared in Example 10), 3-amino-1-benzylpyrrolidine (31 mg, 0.17 mmol), WSC (33 mg, 0.17 mmol), HOBt (23 mg, 0.17 mmol), and triethylamine (36 μl, 0.26 mmol) in CH2Cl2 (4 ml) was stirred at room temperature for 18 h. The reaction mixture was diluted with saturated aqueous NaHCO3 solution and extracted with CH2Cl2. The extracts combined were dried (MgSO4), filtered, and concentrated. The residue was purified by preparative TLC (1 mm thick plate, CH2Cl2/MeOH: 7/1) to give 28 mg (57%) of amide product as colorless oil.

MS (ESI positive) m/z: 563 (M+H)⁺.

A suspension mixture of this oil (28 mg, 0.05 mmol), 10% palladium on activated carbon (10 mg) and EtOH (6 ml) was stirred under hydrogen atmosphere at room temperature for 24 h. Then 5 mg of 10% palladium on activated carbon was added to the reaction mixture and continued the hydrogenation for 24 h. After the removal of the catalyst by filtration, the filtrate was concentrated. The resulting crude oil was purified by preparative TLC (1 mm thick plate, CH2Cl2tMeOH: 7/1) to give 15 mg (64%) of pale brown oil as free form of title compound. This compound showed broadened spectra in proton NMR. This was converted to HCl salt similar to that described in Example 1 to give 15 mg of HCl salt as a white solid.

MS (ESI positive) m/z: 501 (M+H)⁺.

### Example 16

### 2,3-Dihydro-1′-[3-(indol-1-yl)-3-oxopropyl]spiro[1H-indene-1,4′-piperidine]citrate

To a stirred suspension of 2,3-dihydro-1′-[2-(chloroformyl)ethyl]spiro[1H-indene-1,4′-piperidine]hydrochloride (100 mg, 0.32 mmol), indole (75 mg, 0.64 mmol), tetrabutylammonium hydrogen sulfate (54 mg, 0.16 mmol) and powdered NaOH (51 mg, 1.28 mmol) in CH2Cl2 (4 ml) was added triethylamine (67 μl, 0.48 mmol) at room temperature. After 45 minutes stirring, the reaction mixture was quenched with a saturated aqueous NaHCO3 solution and extracted with CH2Cl2. The extracts combined were washed with brine, dried (MgSO4), filtered, and concentrated. The residue was purified by preparative TLC (1 mm thick plate, CH2Cl2/MeOH: 10/1, then purified again using 0.5 mm thick plate, ethyl acetate) to give 7 mg (6%) of colorless oil.

¹H NMR (270 MHz, CDCl3) δ 8.47 (1H, d, J=8.2 Hz), 7.57 (1H, d, J=8;2 Hz), 7.51 (1H, d, J=3.8 Hz), 7.40-7.12 (6H, m), 6.66 (1H, d, J=3.8 Hz), 3.20 (2H, t, J=6.9 Hz), 3.06-2.87 (611, m), 2.40-2.28 (21, m), 2.07-1.91 (4H, m), 1.64-1.54 (2H, m). 7 mg (0.02 mmol) of this oil and citric acid (3.8 mg, 0.02 mmol) was dissolved in CH2Cl2 (1 ml) and MeOH (1 ml) mixture. After 1 h stirring, the mixture solution was concentrated, solidified with ether, and collected by filtration to give 6 mg of title compound as white amorphous solid.

MS (ESI positive) m/z: 359 (M+H)⁺.

### Preparation 9

### 2,3-Dihydro-1′-[3-(2-(S)-carboxyindolin-1-yl)-3-oxopropyl]spiro[1H-indene-1,4′-piperidine]

This was prepared according to the procedure described in Example 10 using 2,3-dihydro-1′-[3-(2-(S)-methoxycarbonylindolin-1-yl)-3-oxopropyl]spiro[1H-indene-1,4′-piperidine] instead of 2,3-dihydro-1′-[3-(2-methoxycarbonylindolin-1-yl)-3-oxopropyl]spiro[1H-indene-1,4′-piperidine]. 300 mg (100%) of title compound was obtained as white solid.

¹H NMR (270 MHz, CDCl3) δ 8.22 (1H, d, J=7.9 Hz), 7.24-7.08 (6H, m), 7.04-6.97 (1H, m), 6.94-6.86 (1H, m), 5.06-4.97 (1H, m), 3.70-3.06 (8H, m), 3.00-2.76 (4H, m), 2.33-2.13 (2H, m), 2.0671.94 (2H, m), 1.68-1.44 (2H, m).

MS (ESI positive) m/z: 405 (M+H)⁺.

### Example 17

### 2,3-Dihydro-1′-{3-[2S)-[[[2-(dimethylamino)ethyl]amino]carbonyl]indolin-1-yl]-3-oxopropyl}spiro[1H-indene-1,4′-piperidine]dicitrate

A mixture of 2,3-dihydro-1′-[3-(2-(S)carboxyindolin-1-yl)-3-oxopropyl]spiro[1H-indene-1,4′-piperidine] (50 mg, 0.124 mmol, this was prepared in Preparation 9), N,N-dimethylethylenediamine (41 μl, 0.37 mmol), WSC (48 mg, 0.25 mmol), HOBt (34 mg, 0.25 mmol), and triethylamine (86 μl, 0.62 mmol) in CH2Cl2 (3 ml) was stirred at room temperature for 18 h. The reaction mixture was diluted with saturated aqueous NaHCO3 solution and extracted with CH2Cl2. The extracts combined were dried (MgSO4), filtered, and concentrated. The residue was purified by preparative TLC (1 mm thick plate, CH2Cl2/MeOH: 5/1) to give 37 mg (63%) of free form of title compound as colorless oil. This compound showed broadened spectra in proton NMR This oil was converted to citric acid salt by mixing with 2 equivalent of citric acid in mixed solvent of CH2Cl2-MeOH followed by concentration.

MS (ESI positive) m/z: 475 (M+H)⁺.

IR(KBr): 3398, 2941, 2712, 1728, 1655, 1595, 1483, 1418, 1215, 760 cm⁻¹

Anal. Calcd for C29H38N4O2-2C6H8O7-H2O: C, 56.16; H, 6.44; N, 6.39. Found: C, 55.82; H, 6.44; N, 6.22.

### Preparation 10

### 2,3-Dihydro-1′-(3-phthalimidopropyl)spiro[1H-indene-1,4′-piperidine]

This was prepared according to the procedure described in Preparation 6 using N-(3-bromopropyl)phthalimide instead of 3-bromopropanol. 1184 mg (71%) of title compound was obtained as yellow solid.

¹H NMR (270 MHz, CDCl3) δ 7.91-7.83 (2H, m), 7.77-7.70 (2H, m), 7.20-7.08(3H, m), 6.97-6.88 (1H, m), 3.80 (2H, t, J=6.8 Hz), 2.88-2.78 (4H, m), 2.47 (2H, t, J=6.9 Hz), 2.11-2.00 (2H, m), 1.98-1.88 (4H, m), 1.74-1.60 (2H, m), 1.48-1.38 (2H, m).

MS (EI, direct) m/z: 374 (M)⁺.

### Preparation 11

### 2,3-Dihydro-1′-[3-(2-nitroanilino)propyl]spiro[1H-indene-1,4′-piperidine]

A mixture of 2,3-dihydro-1′-(3-phthalimidopropyl)spiro[1H-indene-1,4′-piperidine] (1.184 g, 3.16 mmol, this was prepared in preparation 10) and hydrazine hydrate (0.348 g, 6.95 mmol) in MeOH (35 ml) was refluxed with stirring for 2 h. After concentration, the reaction mixture was diluted with aqueous NaHCO3 solution (80 ml) and extracted with CH2Cl2 (50 ml×3). The extracts combined were washed with water (50 ml), dried (NaSO4), filtered, and concentrated to give 381.4 mg (crude yield was 49%) of amine derivative as yellow oil.

¹H NMR (270 MHz, CDCl3) δ 7.23-7.10 (4H, m), 2.93-2.55 (6H, m), 2.50-2.41 (2H, m), 2.20-2.08 (2H, m), 2.05-1.88 (4H, m), 1.75-1.63 (2H, m), 1.60-1.50 (2H, m), 1.40 (2H, br.s).

A mixture of above amine derivative (607 mg, 2.48 mmol), 2-fluoronitrobenzene (0.39 ml, 3.72 mmol), and K2CO3 (514 mg, 3.72 mmol) in MeCN (10 ml) was refluxed with stirring for 16 h. 0.26 ml (2.48 mmol) of 2-fluoronitrobenzene and 342.8 mg (2.48 mmol) of K2CO3 was added to the reaction mixture and reflux was continued for 5 h. The reaction mixture was diluted with water (30 ml) and extracted with CH2Cl2 (40 ml×3). The extracts combined were dried (Na2SO4), filtered, and concentrated to give 1356 mg of crude product which was purified by silica gel column chromatography (n-hexane/acetone: 4/1) to afford 836 mg (92%) of title compound as yellow oil.

¹H NMR (270 MHz, CDCl3) δ 8.32 (1H, br.s), 8.18 (1H, dd, J=1.5, 8.4 Hz), 7.47-7.39 (1H, m), 7.30-7.12 (4H, m), 6.91 (1H, br.d, J=8.4 Hz), 6.63 (1H, ddd, J=1.2, 7.2, 8.4 Hz), 3.46-3.37 (2H, m), 2.96-2.86 (4H, m), 2.53 (2H, t, J=6.8 Hz), 2.23-2.12 (2H, m), 2.07-1.88 (6H, m), 1.60-1.50 (2H, m).

### Example 18

### 2,3-Dihydro-1′-[3-(2-hydroxymethylbenzimidazol-1-yl)-3-oxopropyl]spiro[1H-indene-1,4′-piperidine]citrate

To a stirred solution of nitroaniline derivative (836.3 mg, 2.29 mmol, this was prepared in preparation 11) in mixed solvent of MeOH (4.8 ml), THF (14.4 ml), and water (1.2 ml) was added NH4Cl (367 mg, 6.9 mmol) and Zn powder (1048 mg, 16 mmol) at 0° C. and resulting reaction mixture was stirred at room temperature for 1.5 h. After Celite filtration, the filtrate was concentrated. The resulting residue was diluted with aqueous NaHCO3 solution (50 ml), extracted with CH2Cl2 (40 ml×4). The extracts combined were washed with brine, dried (Na2SO4), filtered, and concentrated to give 797.9 mg of crude phenylenediamine derivative as reddish brown oil.

¹H NMR (270 MHz, CDCl3) δ 7.24-7.10 (4H, m), 6.88-6.63 (4H, m), 3.43 (1H, br.s), 3.22 (211, t, J=6.3 Hz), 3.03-2.94 (2H, m), 2.90 (2H, t, J=7.4 Hz), 2.58 (2H, t, J=6.4 Hz), 2.24-2.11 (2H, m), 2.07-1.84 (8H, m), 1.62-1.50 (2H, m).

A mixture of this phenylenediamine derivative (50.3 mg, 0.15 mmol) and glycolic acid (22.8 mg, 0.3 mmol) in 4N HCl (3 ml) was refluxed with stirring for 22.5 h After cool down to room temperature, the reaction mixture was basified with aqueous 25% NH3 solution and extracted with CH2Cl2 (20 ml×3). The extracts combined were washed with water, dried (Na2SO4), filtered, and concentrated to give 51.6 mg of crude product, which was purified by preparative TLC (CH2Cl2/MeOH: 15/1, 3 times developped) to afford 25.8 mg of product. As this included some impurity, this was purified again by preparative TLC (AcOEt/1-PrOH/25% NH3: 50/10/1) to give 18.8 mg (33%) of free form of title product as pale yellow oil.

¹H NMR (270 MHz, CDCl3) δ 7.79-7.70 (1H, m), 7.44-7.36 (1H, m), 7.31-7.15 (6H, m), 5.01 (2H, s), 4.48 (2H, t, J=6.3 Hz), 3.43 (1H, br.s), 2.87 (2H, t, J=7.3 Hz), 2.82-2.72 (2H, m), 2.34-1.89 (11H, m), 1.57-1.45 (2H, m).

This oil was converted to citric acid salt by mixing with 1 equivalent of citric acid in MeOH (1.5 ml) followed by concentration.

MS (ESI positive) m/z: 376 (M+H)⁺.

IR(KBr): 3396, 2937, 2600, 1717, 1589, 1458, 1209, 1045, 758 cm⁻¹

Anal. Calcd for C24H29N30—C6H8O7-2H2O: C, 59.69; H, 6.85; N, 6.96. Found: C, 59.90; H, 6.51; N, 6.56.

### Example 19

### 2,3-Dihydro-1′-[3-(2-hydroxymethylindolin-1-yl)-3-oxopropyl]spiro[1H-indene-1,4′-piperidine]citrate

This was prepared according to the procedure described in Example 1 using 2-hydroxymethylindoline instead of methyl indoline 2-carboxylate. 126.3 mg (55.9%) of free base as amorphous solid.

This compound showed broadened spectra in proton N except for the following peaks.

¹H NMR (300 MHz, CDCl3) δ 2.89 (2H, t, J=7.3 Hz), 2.40-2.15 (2H, m), 2.05-1.80 (4H, m, including 2H, t, J=7.3 Hz at 2.00 ppm), 1.60-1.45 (2H, m).

This solid was converted to citric acid salt by mixing with 1 equivalent of citric acid in mixed solvent of CH2Cl2 and MeOH, followed by concentration to afford the title product.

¹H NMR (270 MHz, DMSO-d6) δ 8.00 (1H, br.d, J=7.3 Hz), 7.30-7.12 (6H, m), 7.03 (1H, br.t, J=7.3 Hz), 4.704.55 (1H, m), 3.55-2.75 (14H, m, including 2H, t, J=7.1 Hz at 2.89 ppm), 2.63 (2H, d, J=15.2 Hz), 2.53 (2H, d, J=14.5 Hz), 2.13-1.95 (4H, m, including 2H, t, J=7.1 Hz at 2.06 ppm), 1.70-1.60 (2H, m).

MS (ESI positive) m/z: 391 (M+H)⁺.

IR(KBr): 3350, 2941, 2600, 1728, 1641, 1595, 1481, 1420, 1211, 758 cm⁻¹

Anal. Calcd for C25H30N2O2-C6H8O7-2H2O: C, 60.18; H, 6.84; N, 4.53. Found: C, 60.52; H, 6.49; N, 4.49.

### Example 20

### 2,3-Dihydro-1′-[3-(2-methoxymethylindolin-1-yl)-3-oxopropyl]spiro[1H-indene-1,4′-piperidine]hydrochloride

To a stirred mixture of 2,3-dihydro-1′-[3-(2-hydroxymethylindolin-1-yl)-3-oxopropyl]spiro[1H-indene-1,4′-piperidine] (23.7 mg, 0.0607 mmol) and fluobolic acid (48% solution in water, 8.7 μl, 0.0668 mmol) in CH2Cl2 (2 ml) was added dropwise trimethylsilyldiazomethane (2 M solution in hexane, 30.3 μl, 0.0668 mmol) at 0° C. and stirred for 1 h. Then fluobolic acid (48% solution in water, 8.7 μL 0.0668 mmol) and trimethylsilyldiazomethane (2 M solution in hexane, 30.3 μl, 0.0668 mmol) were added to the reaction mixture and stirred at room temperature for 1 h. The reaction mixture was quenched with water and extracted with CH2Cl2. The extracts combined were dried (Na2SO4), filtered, and concentrated. The residue was purified by preparative TLC (acetone/hexane: 1/1) to give 11.2 mg (45.5%) of free form of title compound as an yellow oil.

¹H NMR (300 MHz, CDCl3) δ 8.13 (1H, br.s), 7.25-7.12 (6H, m), 7.04 (1H, dd, J=7.5, 8.4 Hz), 4.65 (1H, br.s), 3.50-3.25 (5H, m, including 3H, s, at 3.31 ppm), 3.03-2.75 (10H, m, including 2H, t, J=7.3 Hz at 2.90 ppm), 2.36-2.24 (2H, m), 2.06-1.93 (4H, m, including 2H, t, J=7.3 Hz at 2.03 ppm), 1.63-1.54 (2H, m).

This was converted to HCl salt similar to that described in Example 1 to give 12.2 mg of HCl salt as a white solid.

MS (ESI positive) m/z: 405 (M+1)⁺.

IR(IBr): 3400, 2900, 2600, 1649, 1597, 1481, 1460, 1420, 1275, 1119, 758 cm⁻¹

### Example 21

### 2,3-Dihydro-1′-{3-[2-(S)-(2-hydroxyethyl)aminocarbonylindolin-1-yl]-3-oxopropyl}spiro[1H-indene-1,4′-piperidine]hydrochloride

This was prepared according to the procedure described in Example 17 using 2-hydroxyethylamine instead of N,N-dimethylethylenediamine and additionally DMF was added as solvent. Solvent ratio of CH2Cl2/THF/DMF was 2/2/1. 10.1 mg (30.4%) of free from of title compound was obtained as amorphous solid.

¹H NMR (270 MHz, CDCl3) δ 8.17 (1H, br.s), 7.26-6.80 (8H, m), 4.94 (1H, br.s), 3.75-2.50 (15H, m), 2.45-2.20 (2H, m), 2.07-1.85 (4H, m, including 2H, t, J=7.1 Hz at 2.01 ppm), 1.63-1.50 (2H, m).

This was converted to HCl salt similar to that described in Example 1 to give 12.2 mg of HCl salt as a white solid.

MS (ESI positive) m/z: 448 (M+H)⁺.

IR(KBr): 3400, 2934, 2700, 1655, 1597, 1481, 1460, 1420, 1271, 1067, 758 cm⁻¹

### Example 22

### 2,3-Dihydro-1′-[3-(2-aminomethylindolin-1-yl)-3-oxopropyl]spiro[1H-indene-1,4′-piperidine]hydrochloride

A mixture of 2,3-dihydro-1′-[3-(2-hydroxymethylindolin-1-yl)-3-oxopropyl]spiro[1H-indene-1,4′-piperidine] (this was prepared in Example 19, 37.5 mg, 0.096 mmol), phthalimide (56.5 mg, 0.384 mmol), N,N,N′-tetramethylazodicarboxamide (66.1 mg, 0.384 mmol) and tributylphosphine (95.7 p, 0.384 mmol) in THF (2 ml) was stirred at room temperature for 1 day. The reaction mixture was concentrated and the residue was purified by preparative TLC (1 mm thick plate×2, CH2Cl2/MeOH: 10:1) to give 106 mg of brown oil. This was purified again by preparative TLC (1 mm thick plate×2, AcOEt/i-PrOH/NH3 solution in EtOH: 100/5/2) to give 57.5 mg of phthalinide derivative as brown oil. A mixture of this oil (57.5 mg) and hydrazine hydrate (18.7 μl, 0.384 mmol) in MeOH (3 ml) was refluxed with stirring for 4 h. After cool down to room temperature, the reaction mixture was concentrated. The resultant solid appeared was removed by filtration. The filtrate was concentrated and the residue was purified by silica gel column chromatography (EtOAc/hexane: 1/5) to give 13.1 mg (35%) of free from of title compound.

¹H NMR (270 MHz, CDCl3) δ 8.90-8.75 (1H, m), 7.25-6.95 (5H, m), 6.72-6.65 (1H, m), 6.60 (1H, d, J=7.8 Hz), 4.16-4.05 (1H, m), 3.52-3.45 (2H, m), 3.25-3.13 (11, m), 2.95-2.75 (4H, m), 2.60-2.50 (2H, m), 2.42-2.35 (2H, m), 2.22-2.09 (2H, m), 1.99 (2H, t, J=7.4 Hz), 1.92-1.77 (2H, m),1.63-1.35 (5H, m).

This was converted to HCl salt similar to that described in Example 1 to give 13.1 mg of HCl salt as a white solid.

MS (ESI positive) m/z: 390 (M+H)⁺.

IR(KBr): 3420, 3269, 2930, 2575, 2480, 1655, 1545, 1466, 1248, 756 cm⁻¹

### Example 23

### 2,3-Dihydro-1′-{3-[2-(S)-(2-aminoethyl)aminocarbonylindolin-1-yl]-3-oxopropyl}spiro[1H-indene-1,4′-piperidine]dihydrochloride

This was prepared according to the procedure described in Example 21 using 2-t-butoxycarbonylaminoethylamine instead of 2-hydroxyethylamine followed by removal of Boc group by treatment of HCl solution in MeOH and basic workup. 18.1 mg (53.1%) of free base was obtained as white amorphous solid.

This compound showed broadened spectra in proton NMR except for the following peaks.

¹H NMR (300 MHz, CDCl3) δ 2.90 (2H, t, J=7.2 Hz), 2.01 (2H, t, J=7.3 Hz), 1.63-1.50 (2H, m).

This was converted to HCl salt similar to that described in Example 1 to give 18 mg of HCl salt as a white solid.

¹H NMR (300 MHz, DMSO-d6) δ 10.50 (1H, br.s), 8.75 (1H, br.s), 8.25-7.85 (4H, m, including 1H, d, J=7.9 Hz), 7.35-7.00 (7H, m), 5.20-5.12 (1H, m), 3.75-2.70 (16H, m), 2.35-2.15 (2H, m), 2.09 (2H, t, J=7.2 Hz), 1.73-1.62 (2H, m).

MS (ESI positive) m/z: 447 (M+H)⁺.

IR(KBr): 3400, 3236, 2941, 2700, 2575, 1655, 1597, 1541, 1481, 1462, 1416, 1269, 970, 758 cm⁻¹.

Anal. Calcd for C27H34N4O2-2HCl-2.9H2O: C, 56.72; H, 7.37; N, 9.80. Found: C, 56.97; H, 7.35; N, 9.75.

### Example 24

### 2,3-Dihydro-1′-{3-[2-(S)-(2-acetamidoethyl)aminocarbonylindolin-1-yl]-3-oxopropyl}spiro[1H-indene-1,4′-piperidine]hydrochloride

A mixture of 2,3dihydro-1′-{3-[2-(S)-(2-aminoethyl)aminocarbonylindolin-1-yl]-3-oxopropyl}spiro[1H-indene-1,4′-piperidine] (this was prepared in Example 23, 55 mg, 0.053 mmol), acetic anhydride (15.1 μl, 0.16 mmol), and 4-dimethylaminopyridine (1.3 mg, 0.011 mmol) in pyridine (3 ml) was stirred at room temperature for 4 h. After evaporation of the pyridine, the residue was diluted with 2N HCl and CH2Cl2. The mixture was extracted with CH2Cl2. The extracts combined were washed with saturated aqueous NaHCO3 solution and brine, dried (Na2SO4), filtered, and concentrated. The residue was purified by preparative TLC (CH2Cl2/MeOH:10/1) to give 23.2 mg (89.2%) of free base as amorphous solid.

This compound showed broadened spectra in proton NMR except for the following peaks.

¹H NMR (270 MHz, CDCl3) δ 7.06 (1H, dd, J=7.0, 7.3 Hz), 2.92 (2H, t, J=7.4 Hz), 2.03 (2H, t, J=7.4 Hz), 1.75-1.50 (2H, m).

This was converted to HCl salt similar to that described in Example 1 to give 23 mg of HCl salt as a white solid.

¹H NMR (300 MHz, DMSO-d6) δ 8.52 (1H, br.s), 8.08 (1H, d, J=7.9 Hz), 7.30-6.95 (8H, m), 5.13-5.05 (1H, m), 3.65-2.45 (17H, m), 2.30-2.00 (4H, m), 1.82 (3H, s), 1.75-1.60 (2H, m).

MS (ESI positive) m/z: 489 (M+H)⁺.

IR(KBr): 3400, 3267, 2936, 2700, 2573, 1655, 1545, 1481, 1416, 1246, 746 cm⁻¹.

Anal. Calcd for C29H36N4O3-HCl-2.2H2O: C, 61.68; H, 7.39; N, 9.92. Found: C, 61.60; H, 7.33; N, 9.89.

### Example 25

### 2,3-Dihydro-1′-{3-[2-(S)-(2-methanesulfonamidoethyl)aminocarbonylindolin-1-yl-3-oxopropyl}spiro[1H-indene-1,4′-piperidine]hydrochloride

A mixture of 2,3-dihydro-1′-{3-[2-(S)-(2-aminoethyl)aminocarbonylindolin-1-yl]-3-oxopropyl}spiro[1H-indene-1,4′-piperidine] (this was prepared in Example 23, 55.2 mg, 0.052 mmol), mesyl chloride (6 μl, 0.077 mmol), and triethylamine (21.6 μl, 0.155 mmol) in CH2Cl2 (2 ml) was stirred at room temperature for 1 day. The reaction mixture was diluted with saturated NaHCO3 aqueous solution and extracted with CH2Cl2. The extracts combined were dried (Na2SO4), filtered, and concentrated. The residue was purified by preparative TLC (CH2Cl2/MeOH:10/1) to give 10.5 mg (38.7%) of free base as amorphous solid.

This compound showed broadened spectra in proton NMR except for the following peaks.

¹H NMR (270 MHz, CDCl3) δ 7.06 (1H, dd, J=7.3, 7.8 Hz), 2.90 (3H, s), 2.03 (2H, t, J=7.4 Hz), 1.75-1.50 (2H, m).

This was converted to HCl salt similar to that described in Example 1 to give 10.5 mg of HCl salt as a white solid.

¹H NMR (300 MHz, CDCl3), 10.22 (1H, br.s), 8.15 (1H, d, J=7.2 Hz), 7.90-7.00 (10H, m), 5.30-5.05 (1H, m), 4.30-2.85 (17H, m, including 3H, s, at 2.96 ppm), 2.75-2.45 (2H, m), 2.40-1.90 (3H, m), 1.85-1.65 (2H, m).

MS (ESI positive) m/z: 525 (M+H)⁺.

IR(KBr): 3400, 2936, 2700, 2573, 1655, 1483, 1313, 1151, 758 cm⁻¹.

### Preparation 12

### Methyl 2-(benzothiazol-2-one-1-yl)-4-hydroxybutyrate

To a stirred solution of 2-hydroxybenzothiazole (300 mg, 1.98 mmol) in DMF (5 ml) was added NaH (60% oil suspension, 160 mg, 3.97 mmol) at room temperature. To this mixture was added α-bromo-γ-butyrolactone (660 mg, 3.97 mmol) and resulting reaction mixture was stirred at room temperature for 1 h, and at 60° C. for 30 minutes. Then NaH (80 mg, 1.98 mmol) and α-bromo-γ-butyrolactone (330 mg, 1.98 mmol) was added to the reaction mixture and stirred at 60° C. for 1 h. The reaction mixture was poured into aqueous NaHCO3 solution and extracted with ethyl acetate. The extracts combined were dried (MgSO4) and concentrated. The residue was purified by silica gel column chromatography (hexane/ethyl acetate:3/2) to give 0.35 g (75%) of lactone derivative as white solid.

¹H NMR (300 MHz, CDCl3) δ 7.47 (1H, dd, J=0.9, 7.6 Hz), 7.32 (1H, ddd, J=1.3, 7.5, 7.7 Hz), 7.20 (1H, ddd, J=1.1, 7.7, 7.7 Hz), 6.93 (1H, d, J=8.0 Hz), 5.45-5.30 (1H, m), 4.71 (1H, ddd, J=2.4, 9.2, 9.3 Hz), 4.46 (1H, ddd, J=7.0, 9.3, 10.1 Hz), 2.88-2.62 (2H, m).

To a stirred suspension of the above lactone derivative (0.39 g, 1.66 mmol) in MeOH (12 ml) was added c-H2SO4 (1 ml) and the reaction mixture was stirred at 60° C. for 2 h. The reaction mixture was poured into water and extracted with ethyl acetate. The extracts combined were washed with aqueous NaHCO3 solution and brine, dried (MgSO4), filtered, and concentrated. The residue was purified by silica gel column chromatography (CH2Cl2/MeOH: 10/1) followed by preparative TLC (1 mm thick plate, CH2Cl2/MeOH: 20/1) to give 173 mg (39%) of the title compound as a colorless oil.

¹H NMR (270 MHz, CDCl3) δ 7.48 (1H, dd, J=1.3, 7.7 Hz), 7.30 (1H, ddd, J=1.5, 7.7, 7.9 Hz), 7.19 (1H, ddd, J=1.1, 7.6, 7.7 Hz), 7.00 (1H, d, J=7.9 Hz), 5.47 (1H, dd, J=4.6, 10.7 Hz), 3.80-3.74 (1H, m), 3.74 (3H, s), 3.50-3.40 (1H, m), 2.67-2.53 (1H, m), 2.35-2.22 (1H, m), 2.06-1.97 (1H, m).

### Preparation 13

### 2,3-Dihydro-1′-[3-(benzothiazol-2-one-1-yl)-3-methoxycarbonylpropyl]spiro[1H-indene-1,4′-piperidine]

To a stirred solution of methyl 2-(benzothiazol-2-one-1-yl)-4-hydroxybutyrate (0.21 g, 0.79 mmol) and triethylamine (0.14 ml, 1.03 mmol) in CH2Cl2 (5 ml) was added mesyl chloride (67 μl, 0.86 mmol) at 0° C. After 15 min stirring, the reaction mixture was poured into aqueous NaHCO3 solution and extracted with CH2Cl2. The extracts combined were dried (MgSO4), filtered, and concentrated. To this residue was added toluene and concentrated again to give 0.30 g of crude mesylate as colorless oil.

¹H NMR (270 MHz, CDCl3) δ 7.47 (1H, br.d, J=7.7 Hz), 7.35-7.15 (2H, m), 7.19 (1H, br.d, J=8.2 Hz), 5.37-5.27 (1H, m), 4.454.35 (1H, m), 4.17-4.07 (1H, m), 3.75 (3H, s), 2.94 (3H, s), 2.90-2.78 (1H, m), 2.65-2.50 (1H, m).

A mixture of this oil (0.30 g, 0.79 mmol), 2,3-dihydrospiro[1H-indene-1,4′-piperidine]hydrochloride (0.194 g, 0.87 mmol), and diisopropylethylamine (0.31 g, 2.37 mmol) in MeOH (10 ml) was stirred at 60° C. for 14 h and at 80° C. for4 h. The reaction mixture was concentrated, then diluted with CH2Cl2, wasahed with aqueous NaHCO3 solution, dried (MgSO4), filtered, and concentrated. The residue was purified by silica gel column chromatography (CH2Cl2/MeOH: 30/1) to give 165 mg (48%) of title compound as colorless oil.

¹H NMR (270 MHz, CDCl3) δ 7.45 (1H, dd, J=1.6, 8.2 Hz), 7.33-7.26 (1H, m), 7.22-7.12 (6H, m), 5.47-5.36 (1H, m), 3.74 (3H, s), 2.90-2.82 (3H, m, including 2H, t, J=7.1 Hz at 2.86 ppm), 2.65-2.50 (2H, m), 2.42-2.25 (3H, m), 2.15-2.05 (2H, m), 1.95 (2H, t, 3=7.3 Hz), 1.92-1.65 (2H, m), 1.60-1.37 (2H, m).

### Example 26

### 2,3-Dihydro-1′-[3-(benzothiazol-2-one-1-yl)-3-hydroxymethylpropyl]Spiro[1H-indene-1,4′-piperidine]hydrochloride

To a stirred solution of 2,3-dihydro-1′-[3-(benzothiazol-2-one-1-yl)-3methoxycarbonyl-propyl]spiro[1H-indene-1,4′-piperidine] (40 mg, 0.092 mmol) in THF (2 ml) was added LiAlH4 (3.5 mg, 0.092 mmol) at 0° C. After 30 min stirring, LiAlH4 (7 mg, 0.184 mmol) was added to the reaction mixture and stirring was continued another 10 min at 0° C. The reaction mixture was quenched with 15 μl of water, 15 μl of 2N NaOH solution, and 45 μl of water, then the resulting mixture was stirred for 20 min at room temperature. After Celite filtration, the filtrate was concentrated. The residue was purified by preparative TLC (CH2Cl2/MeOH: 10/1, then ethyl acetate) to give 8 mg (22%) of free form of title compound as white solid.

¹H NMR (270 MHz, CDCl3) δ 7.44-7.40 (1H, m), 7.34-7.30 (2H, m), 7.24-7.12 (6H, m), 4.65-4.40 (1H, m), 4.20 (1H, dd, J=6.4, 11.7 Hz), 3.95 (1H, dd, J=7.6, 11.8 Hz), 3.16-3.02 (1H, m), 2.90 (2H, t, J=7.2 Hz), 2.85-2.75 (1H, m), 2.62-2.48 (3H, m), 2.39-2.26 (1H, m), 2.20-2.08 (1H, m), 2.08-1.84 (5H, m, including 2H, t, J=7.4 Hz at 2.00 ppm), 1.65-1.50 (2H, m).

This was treated with HCl solution in MeOH followed by concentration to give 8 mg of HCl salt as white amorphous solid.

MS (ESI positive) m/z: 409 (M+H)⁺.

### Preparation 14

### 2,3-Dihydro-1′-[3-(benzothiazol-2-one-1-yl)-3-carboxypropyl]spiro[1H-indene-1,4′-piperidine]

A mixture of 2,3-dihydro-1′-[3-(benzothiazol-2-one-1-yl)-3-methoxycarbonylpropyl]spiro[1H-indene-1,4′-piperidine] (110 mg, 0.25 mmol) and 2N NaOH solution (0.5 ml, 1 mmol) in THF (2 ml) and MeOH (1 ml) was stirred at room temperature for 16 h. The reaction mixture was diluted with ethyl acetate, washed with HCl solution and brine, dried (MgSO4), filtered, and concentrated to give 103 mg (96%) of title compound as white solid.

¹H NMR (300 MHz, DMSO-d6) δ 7.73 (1H, d, J=7.9 Hz), 7.46-7.36 (2H, m), 7.30-7.05 (5H, m), 5.45-5.35 (1H, m), 3.55-2.95 (9H, m), 2.86 (2H, t, J=7.1 Hz), 2.80-2.63 (1H, m), 2.25-1.95 (4H, m, including 2H, t, J=7.5 Hz at 2.02 ppm), 1.70-1.56 (2H, m).

MS(EI direct) m/z: 422(M)⁺.

### Example 27

### 2,3-Dihydro-1′-[3-(benzothiazol-2-one-1-yl)-3-(N,N-dimethylaminocarbonyl) propyl]spiro[1H-indene-1,4′-piperidine]hydrochloride

This was prepared according to the procedure described in Example 11 using 2,3-dihydro-1′-[3-(enzothiazol-2-one-1-yl)₃-carboxypropyl]spiro[1H-indene-1,4′-piperidine] instead of 2,3-dihydro-1′-[3-(2-carboxyindolin-1-yl)-3-oxopropyl]spiro[1H-indene-1,4′-piperidine]. Yield was 30 mg (71%). Product was colorless amorphous solid.

¹H NMR (270 MHz, CDCl3) δ 7.55-7.49 (1H, m), 7.46-7.41 (1H, m), 7.30-7.09 (6H, m), 5.72-5.62 (1H, m), 2.96 (3H, s), 2.95 (3H, s), 2.88-2.73 (4H, m, including 2H, t, J=7.2 Hz at 2.85 ppm), 2.50-2.22 (4H, m), 2.20-1.80 (5H, m, including 2H, t, J=7.4 Hz at 1.93 ppm), 1.70-1.55 (1H, m), 1.50-1.35 (2H, m).

This was treated with HCl solution in MeOH followed by concentration to give 30 mg of HCl salt as white amorphous solid.

MS (ESI positive) m/z: 450 (M+H)⁺.

IR(KBr): 3439, 2932, 2563, 1655, 1589, 1472, 758 cm⁻¹

Anal. Calcd for C26H31N3O2S—HCl—H2O: C, 61.95; H, 6.80; N, 8.34. Found: C, 62.33; H, 7.00; N, 7.89.

### Example 28

### 2,3-Dihydro-1′-[3-(benzothiazol-2-one-1-yl)-3-(2-N,N-dimethylaminoethylaminocarbonyl)propyl]spiro[1H-indene-1,4′-piperidine]hydrochloride

This was prepared according to the procedure described in Example 27 using N,N-dimethylethylenediamine instead of dimethylamine hydrochloride. Yield was 30 mg (80%). Product was colorless oil.

¹H NMR (270 MHz, CDCl3) δ 7.45 (1H, br.d, J=7.7 Hz), 7.32-7.10 (7H, m), 6.77 (1H, br.s), 5.41 (1H, dd, J=5.3, 9.0 Hz), 3.40-3.20 (2H, m), 2.90-2.75 (3H, m, including 2H, t, J=7.4 Hz at 2.85 ppm), 2.70-2.50 (2H, m), 2.45-1.75 (16H, m, including 6H, s at 2.05 ppm and 2H, t, J=7.2 Hz at 1.93 ppm), 1.70-1.30 (3H, m).

This was treated with HCl solution in MeOH followed by concentration to give 32 mg of HCl salt as white amorphous solid.

MS (ESI positive) m/z: 493 (M+H)⁺.

IR(KBr): 3408, 2934, 2691, 1670, 1537, 1472, 758 cm⁻¹

Anal. Calcd for C28H36N4O2S-2HCl-1.2H2O: C, 57.27; H. 6.93; N, 9.54. Found: C, 57.623; H, 7.31; N, 9.07.

### Example 29

### 2,3-Dihydro-1-[3-(3-ethylbenzimidazol-2-one-1-yl)propyl]spiro[1H-indene-1,4′-piperidine]hydrochloride

NaH (60% oil suspension, 11.7 mg, 0.293 mmol) was washed with hexane (2 ml×2) and decanted, then DMF (1 ml) was added. To a stirred this suspension was added a solution of 2,3-dihydro-1′-[3-(benzimidazol-2-one-1-yl)propyl]spiro[1H-indene-1,4′-piperidine] (66.1 mg, 0.193 mmol) in DMF (1.5 ml) at room temperature. After stirring for 0.5 h, a solution of iodoethane (57.1 mg, 0.366 mmol) was dropwisely added to the reaction mixture at 0° C. and the resulting mixture was stirred at room temperature for 19 h. The reaction mixture was diluted with saturated aqueous NaHCO3 solution and extracted with ethyl acetate. The extracts combined were washed with water, dried (Na2SO4), filtered, and concentrated to give 67.5 mg of crude product, which was purified by preparative TLC (CH2Cl2/MeOH: 15/1) to give 30.5 mg (43%) of free form of title compound as pale yellow oil.

¹H NMR (270 MHz, CDCl3) δ 7.25-6.98 (8H, m), 4.01-3.91 (4H, m), 2.92-2.82 (4H, m), 2.46 (2H, t, =6.9 Hz), 2.20-2.07 (2H, m), 2.06-1.76 (6H, m), 1.58-1.48 (2H, m), 1.35 (3H, t, J=7.2 Hz).

This was converted to citric acid salt according to the procedure described in Example 34 to give 38.3 mg of citrate as white amorphous solid.

MS (ESI positive) m/z: 390 (M+H)⁺.

IR(KBr): 3416, 2937, 2584, 1686, 1492, 1420, 1192, 756 cm⁻¹

Anal. Calcd for C25H31N30-C6H8O7-1.2H2O: C, 61.72; H, 6.92; N, 6.97. Found: C, 61.83; H, 6.94; N, 6.51.

### Example 30

### 2,3-Dihydro-1′-[3-(2-acetamidobenzimidazol-1-yl)propyl]spiro[1H-indene-1,4′-piperidine]citrate

To a stirred solution of 2,3-Dihydro-1′-[3-(2-aminoanilino)propyl]spiro[1H-indene-1,4′-piperidine] (this was prepared in the first step of Example 18, 105.7 mg, 0.315 mmol) in THF (1 ml) was added a solution of cyanogen bromide (33.4 mg, 0.315 mmol) in mixed solvent of THF (1 ml) and water (1 ml) at room temperature. After 16.5 h, the reaction mixture was basified by 25% NH3 solution in water at 0° C. and extracted with CH2Cl2. The extracts combined were dried (Na2SO4), filtered, and concentrated to give 114.3 mg of crude product. To a solution of this compound (53.1 mg, 0.147 mmol) in CH2Cl2 (1.5 ml) was added catalytic amount of 4-dimethylaminopyridine, triethylamine (41 μl, 0.726 mmol), and a solution of acetyl chloride (17.3 mg, 0.221 mmol) in CH2Cl2 (1.5 ml) at 0° C. After 2 h stirring, the reaction mixture was warmed to room temperature and stirred another 3 h. The reaction mixture was quenched with saturated aqueous NaHCO3 solution (10 ml) and extracted with CH2Cl2. The extracts combined were washed with brine, dried (Na2SO4), filtered, and concentrated. The residue was purified by preparative LC (CH2Cl2/MeOH: 15/1) to afford 7.6 mg (13%) of free form of title compound as pale yellow oil.

¹H NMR (270 MHz, CDCl3) δ 7.35-7.10 (8H, m), 4.25-4.15 (4H, m), 2.96-2.82 (8H, m), 2.22-1.96 (7H, m, including 3H, s, at 2.17 ppm), 1.75-1.50 (3H, m).

MS (EI direct) m/z: 402 (M⁺), 227, 189.

This was converted to citric acid salt according to the procedure described in Example 34 to give 4.6 mg of citrate as white amorphous solid.

Anal. Calcd for C25H30N40-C6H8O7-1.5H2O: C, 59.89; H, 6.65; N, 9.01. Found: C, 60.15; H, 6.58; N, 8.76.

### Example 31

### 2,3-Dihydro-1′-{3-[3-(2-hydroxyethyl)benzimidazol-2-one-1-yl]propyl}spiro[1H-indene-1,4′-piperidine]citrate

This was prepared according to the procedure described in Example 29 using t-butyldimethylsilyloxyethyl bromide instead of iodoethane followed by deprotection using tetrabutylammonium fluoride in THF. Yield was 48.4 mg (57%). Product was colorless oil.

¹H NMR (270 MHz, CDCl3) δ 7.23-6.99 (8H, m), 4.09-3.92 (6H, m), 2.92-2.80 (4H, m, including 2H, t, J=7.2 Hz), 2.45 (2H, t, J=7.1 Hz), 2.19-2.07 (2H, m), 2.05-1.83 (6H, m), 1.75 (1H, br.s), 1.58-1.46 (2H, m).

MS (EI direct) m/z: 405 (M⁺), 375, 275, 200.

This was converted to citric acid salt according to the procedure described in Example 34 to give 11.6 mg of citrate as white amorphous solid.

IR(KBr): 3406, 2939, 2579, 1686, 1495, 1416, 1192, 756 cm⁻¹

Anal. Calcd for C25H31N3O2-C6H8O7-2H2O: C, 58.76; H, 6.84; N, 6.63. Found: C, 58.93; H, 6.62; N, 6.33.

### Example 32

### 2,3-Dihydro-1′-{3-[3-(2-aminoethyl)benzimidazol-2-one-1-yl]propyl}spiro[1H-indene-1,4′-piperidine]citrate

This was prepared according to the procedure described in Example 29 using N-(2-bromoethyl)phthalimide instead of iodoethane followed by deprotection using hydrazine hydrate in MeOH. Yield was 20.1 mg (10.8%). Product was colorless oil.

¹H NMR (270 MHz, CDCl3) δ 7.23-7.02 (8H, m), 4.02-3.92 (4H, m), 3.08 (2H, t, J=6.2 Hz), 2.92-2.80 (4H, m, including 2H, t, J=7.4 Hz at 2.88 ppm), 2.46 (2H, t, J=6.9 Hz), 2.20-2.07 (2H, m), 2.06-1.83 (6H, m), 1.58-1.48 (2H, m), 1.26 (2H, br.s),.

MS (EI direct) m/z: 404 (M⁺), 277, 200.

This was converted to citric acid salt according to the procedure described in Example 34 to give 7.5 mg of citrate as white amorphous solid.

Anal. Calcd for C25H32N40-C6H8O7-3H2O: C, 57.22; H, 7.13; N, 8.61. Found: C, 57.35; H, 6.82; N, 8.45.

### Example 33

### 2,3-Dihydro-1′-{3-[3-(2-acetamidoethyl)benzimidazol-2-one-1-yl]propyl}spiro[1H-indene-1,4′-piperidine]citrate

To a stirred solution of 2,3-dihydro-1′-{3-[3-(2-aminoethyl)benzimidazol-2-one-1-yl)propyl}spiro[1H-indene-1,4′-piperidine] (12.7 mg, 0.031 mmol, this was prepared in Example 32) in CH2Cl2 (1.5 ml) was added catalytic amount of 4-dimethylaminopyridine and triethylamine (7.9 μl, 0.056 mmol) followed by addition of acetyl chloride (2.6 μl, 0.037 mmol) at 0° C. After 1 h stirring at 0° C. and 2 h stirring at room temperature, acetyl chloride (2.6 μl, 0.037 mmol) and triethylamine (7.9 μl, 0.056 mmol) were added to the reaction mixture at 0° C. After 1 h stirring at 0° C. and 2 h stirring at room temperature, the reaction mixture was quenched with saturated aqueous NaHCO3 solution and extracted with CH2Cl2. The extracts combined were washed with brine, dried (Na2SO4), and concentrated to give 14 mg of crude product, which was purified by preparative TLC (CH2Cl2/MeOH: 10/1) to afford 12.5 mg (90%) of free form of title compound as colorless oil.

¹H NMR (270 MHz, CDCl3) δ 7.24-7.02 (8H, m), 6.40 (1H, br.s), 4.07 (2H, t, J=5.6 Hz), 3.98 (2H, t, J=6.9 Hz), 3.64-3.55 (2H, m), 2.92-2.80 (4H, m, including 2H, t, J=7.3 Hz at 2.89 ppm), 2.46 (2H, t, J=6.8 Hz), 2.20-2.07 (2H, m), 2.05-1.83 (9H, m, including 3H, s, at 1.95 ppm), 1.60-1.46 (2H, m).

This was converted to citric acid salt according to the procedure described in Example 34 to give 8.7 mg of citrate as white amorphous solid.

MS (ESI positive) m/z: 447 (M+H)⁺.

IR(KBr): 3400, 2943, 2579, 1690, 1495, 1418, 1198, 754 cm⁻¹

Anal. Calcd for C27H34N4O2-C6H8O7-1.9H2O: C, 58.90; H, 6.86; N, 8.33.

Found: C, 59.22; H, 6.57; N, 7.93

### Example 34

### 2,3-Dihydro-1′-[3-(2-(S)-N-methylaminocarbonylindolin-1-yl)-3-oxopropyl]spiro[1H-indene-1,4′-piperidine]citrate

This was prepared according to the procedure described in Example 17 using N-methylamine hydrochloride instead of N,N-dimethylethylenediamine. Yield was 32 mg (62%). Product was colorless amorphous solid.

This compound showed broadened spectra in proton NMR except for the following peaks.

¹H NMR (270 MHz, CDCl3) δ 2.79 (3H, d, J=4.8 Hz), 2.35-2.20 (2H, m), 2.05-1.85 (4H, m), 1.62-1.50 (2H, m).

This was dissolved in mixed solvent of CH2Cl2 (1 ml) and MeOH (1 ml) followed by addition of citric acid (15 mg, 0.0766 mmol) and resulting mixture was stirred for 2 h. After concentration, the residue was solidified by adding CH2Cl2-hexane. The resulting solid was collected by filtration and washed with ether to give 37 mg of citrate as white amorphous solid.

MS (ESI positive) m/z: 418 (M+H)⁺.

IR(KBr): 3362, 2937, 2586, 1728, 1653, 1597, 1483, 1411, 758 cm⁻¹

Anal. Calcd for C26H31N3O2-C6H8O7-2.3H2O: C, 59.03; H, 6.75; N, 6.45. Found: C, 59.41; H, 6.49; N, 5.87

### Example 35

### 2,3-Dihydro-1′-[3-(2-(S)-N,N-dimethylaminocarbonylindolin-1-yl)-3-oxopropyl]spiro[1H-indene-1,4′-piperidine]citrate

This was prepared according to the procedure described in Example 17 using N,N-dimethylamine hydrochloride instead of N)V-dimethylethylenediamine. Yield was 24 mg (45%). Product was colorless amorphous solid.

¹H NMR (270 MHz, CDCl3) δ 8.30 (0.4H, br.d, J=8.2 Hz), 7.32-7.08 (6.6H, m), 7.03-6.96 (1H, m), 5.54-5.42 (0.6H, m), 5.33-5.21 (0.4H, m), 3.77-3.60 (0.4H, m), 3.55-3.38 (0.6H, m), 3.03-2.80 (14H, m, including 1.2H, s, at 3.00 ppm, 1.8H, s, at 2.98 ppm, 1.2H, s, at 2.93 ppm, and 1.8H, s, at 2.90 ppm), 2.70-2.20 (3H, m), 2.10-1.90 (4H, m), 1.65-1.50 (2H, m).

This was converted to citric acid salt according to the procedure described in Example 34 to give 30 mg of citrate as white amorphous solid.

MS (ESI positive) m/z: 432 (M+H)⁺.

IR(KBr): 3416, 2936, 2561, 1728, 1655, 1597, 1485, 1406, 758 cm⁻¹

Anal. Calcd for C27H33N3O2-C6H8O7-H2O: C, 61.77; H, 6.75; N, 6.55. Found: C, 61.96; H, 6.84; N, 6.24

### Example 36

### 2,3-Dihydro-1′-{3-[2-(S)-(4-morpholinecarbonyl)indolin-1-yl]-3-oxopropyl}spiro[1H-indene-1,4′-piperidine]citrate

This was prepared according to the procedure described in Example 17 using morpholine instead of N,N-dimethylethylenediamine. Yield was 37 mg (63%). Product was colorless amorphous solid.

¹H NMR (270 MHz, CDCl3) δ 8.29 (0.41H, br.d, J=8.0 Hz), 7.35-6.96 (7.6H, m), 5.50-5.30 (1H, m), 3.90-3.40 (10H, m), 3.20-2.70 (8H, m), 2.65-2.20 (3H, m), 2.20-1.90 (4H, m), 1.68-1.50 (21H, m).

This was converted to citric acid salt according to the procedure described in Example 34 to give 45 mg of the title product as white amorphous solid.

MS (ESI positive) m/z: 474 (M+H)⁺.

IR(KBr): 3414, 2930, 2573, 1728, 1655, 1597, 1485, 1437, 1236, 1115, 758 cm⁻¹

Anal. Calcd for C29H35N3O3-C6H8O7-1.5H2O: C, 60.68; H, 6.69; N, 6.07. Found: C, 60.62; H, 6.66; N, 5.71

### Preparation 15

### 2,3-Dihydro-1′-[3-[(2R)-2-(aminocarbonyl)-2,3-dihydro-1H-indol-1-yl]-3-oxopropyl]spiro[1H-indene-1,4′-piperidine]and 2,3-Dihydro-1′-[3-[(2S)-2-(aminocarbonyl)-2,3-dihydro-1H-indol-1-yl]-3-oxopropyl]spiro[1H-indene-1,4′-piperidine]

Racemic 2,3-Dihydro-1′-[3-[2-(aminocarbonyl)-2,3-dihydro-1H-indol-1-yl]-3-oxopropyl]spiro[1H-indene-1,4′-piperidine] (60 mg, 0.15 mmol, this was prepared in Example 13) was separated by preparative HPLC on chiral stationary phase (DAICEL CHIRALPAK AS, 20×250 mm, hexane/EtOH/Et₂NH:50/50/0.1 as eluent, 6 ml/min.).

Former fraction was (R)-enantiomer, obtained with e.e.>99% (HPLC).

Later fraction was (S)-enantiomer, obtained with e.e.>99% (BPLC).

(S)-Enantiomer was also prepared according to the procedure described in Example 14 using (2S)-indolinecarboxamide instead of methyl (2S)-indolinecarboxylate. Yield was 82 mg (59%). Product was pale brown amorphous solid.

(S)-Enantiomer showed broadened spectra in proton NMR except for the following peaks.

¹H NMR (270 MHz, CDCl3) δ 2.40-2.20 (2H, m), 2.10-1.85 (4H, m), 1.75-1.50 (2H, m).

MS (ESI positive) m/z: 404 (M+H)⁺.

### Example 37

### 2,3-Dihydro-1′-1-[(2R)-2-(aminocarbonyl)-2,3-dihydro-1H-indol-1-yl]-3-oxopropyl]spiro[1H-indene-1,4′-piperidine]citrate

2,3-Dihydro-1′-[3-[(2R)-2-(aminocarbonyl)-2,3-dihydro-1H-indol-1-yl]-3-oxopropyl]spiro[1H-indene-1,4′-piperidine] (20 mg) was converted to citric acid salt according to the procedure described in Example 34 to give 28 mg of the title product as white amorphous solid.

MS (ESI positive) m/z: 404 (M+H)⁺.

### Example 38

### 2,3-Dihydro-1′-[3-[(2S)-2-(aminocarbonyl)-2,3-dihydro-1H-indol-1-yl]-3-oxopropyl]spiro[1H-indene-1,4′-piperidine]citrate

2,3-Dihydro-1′-[3-[(2S)-2-(aminocarbonyl)-2,3-dihydro-1H-indol-1-yl]-3-oxopropyl]spiro[1H-indene-1,4′-piperidine] (27 mg) was converted to citric acid salt according to the procedure described in Example 34 to give 33 mg of the title product as white amorphous solid.

MS (ESI positive) m/z: 404 (M+H)⁺

### Example 39

### 2,3-Dihydro-1′-13-(2-hydroxymethylindolin-1-yl)propyl]spiro[1H-indene-1,4′-piperidine]citrate

To a stirred solution of 2,3-Dihydro-1′-[3-(2-hydroxymethylindolin-1-yl)-3-oxopropyl]spiro[1H-indene-1,4′-piperidine] (0.13 g, 0.34 mmol, this was prepared in Example 19) in THF (5 ml) was added LiAlH4 (40 mg, 1.05 mmol) at 0° C. The resulting reaction mixture was stirred at the same temperature for 2.5 h., quenched by the following addition with water (50 μl), 2N NaOH (501 μl), and water (150 μl), and stirred for 30 min. The resulting mixture was filtered through a pad of celite, and the filtrate was concentrated in vacuo. The residue was purified by preparative TLC (1 mm thick silica gel plate: CH2Cl2/MeOH: 10/1) to afford 8.8 mg (7%) of free base as a pale yellow amorphous.

¹H NMR (300 MHz, CDCl3) δ 7.35-7.00 (6H, m), 6.66 (1H, t, J=7.3 Hz), 6.49 (1H, d, J=7.3 Hz), 3.95-3.70 (3H, m), 3.57-3.45 (1H, m), 3.27-3.15 (1H, m), 3.13-2.85 (6H, m), 2.78-2.65 (1H, m), 2.43-2.22 (2H, m), 2.20-1.82 (8H, m), 1.65-1.48 (2H, m).

This was converted to citric acid salt according to the procedure described in Example 34 to give 10 mg of the title product as a white amorphous solid.

MS (ESI positive) m/z: 377 (M+H)⁺.

### Example 40

### 2,3-Dihydro-1′-[3-(3,4-dihydro-[(2H)quinolinyl)-3-oxopropyl]spiro [H-indene-1,4′-piperidine]hydrochloride

This was prepared according to the procedure described in Example 1 using 1,2,3,4-tetrahydroquinoline instead of methyl indoline-2-carboxylate. 14 mg (36%) of free form of title compound was obtained as colorless oil.

¹H NMR (300 MHz, CDCl3) δ 7.24-7.08 (8H, m), 3.81 (2H, t, J=6.6 Hz), 2.87 (2H, t, J=7.5 Hz), 2.84-2.72 (6H, m), 2.73 (2H, t, J=6.6 Hz), 2.24-2.12 (2H, m), 2.03-1.82 (61H, m), 1.56-1.46 (2H, m).

This was converted to HCl salt similar to that described in Example 1 to afford 10 mg of the title product as white amorphous solid.

MS (ESI positive) m/z: 375 (M+H)⁺.

IR(KBr): 3422, 2937, 2559, 1655, 1490, 1398, 1203, 750 cm⁻¹

### Example 41

### 2,3-Dihydro-1′-[3-[2-(aminocarbonyl)-2,3-dihydro-4H-1,4-benzothiazin-4-yl]-3-oxopropyl]spiro[1H-indene-1,4′-piperidine]citrate

This was prepared according to the procedure described in Example 1 using 3,4-dihydro-2H-1,4-benzothiazine-2-carboxamide (this was prepared according to known procedure: Butler Richard C. M. et al, J. Heterocycl. Chem. 1985, 22, 177) instead of methyl indoline-2-carboxylate. 3 mg (4%) of free form of title compound was obtained as pale brown oil.

This compound showed broadened spectra in proton NMR.

This was converted to citric acid salt according to the procedure described in Example 34 to give 3 mg of the title product as a white solid.

MS (ESI positive) m/z: 436 (M+H)⁺.

### Preparation 16

### 2,3-Dihydro-1′-[3-[(2S)-2-[[((3R)-1-benzyl-3-pyrrolidinyl)amino]carbonyl]-2,3-dihydro-1H-indol-1-yl]-3-oxopropyl]spiro[1H-indene-1,4′-piperidine]

This was prepared according to the procedure described in Example 17 using (3R)-1-benzyl-3-aminopyrrolidine instead of N,N-dimethylethylenediamine. 490 mg (88%) of title product was obtained as a pale yellow solid.

This compound showed broadened spectra in proton NMR.

MS (ESI positive) m/z: 563 (M+H)⁺.

### Example 42

### 2,3-Dihydro-1′-[3-[(2S)-2-[[((3R)-1H-3-pyrrolidinyl)amino]carbonyl]-2,3-dihydro-1H-indol-1-yl]-3-oxopropyl]spiro[1H-indene-1,4′-piperidine]citrate

A mixture of 2,3-dihydro-1′-[3-[(2S)-2-[[((3R)-1-benzyl-3-pyrrolidinyl)amino]carbonyl]-2,3-dihydro-1H-indol-1-yl]-3-oxopropyl]spiro[1H-indene-1,4′-piperidine] (490 mg, 0.87 mmol), 2N HCl (2 ml), and 10% Pd—C (100 mg) in MeOH (10 ml) was stirred at room temperature under hydrogen atmosphere (4 atm) for 8 h. The reaction mixture was filtered through a pad of celite and the filtrate was concentrated in vacuo. The resulting residue was purified by preparative TLC (1 mm thick silica gel plate: CH2Cl2/MeOH/b 25% NH3: 100/10/1) to afford 296 mg (72%) of free base as a pale yellow amorphous.

¹H NMR (270 MHz, CDCl3) δ 8.35-8.23 (0.3H, m), 7.40-6.70 (7.7H, m), 5.254.85 (1H, m), 4.40-4.20 (1H, m), 3.70-2.50 (16H, m), 2.35-1.85 (5H, m), 2.00 (2H, t, J=7.3 Hz), 1.75-1.45 (3H, m).

This product (99 mg) was converted to citric acid salt according to the procedure described in Example 34 to give 137 mg of the title product as a white amorphous solid.

MS (ESI positive) m/z: 473 (M+H)⁺.

IR(KBr): 3416, 3022, 2941, 1717, 1668, 1597, 1483, 1416, 1269, 758 cm⁻¹

### Example 43

### 2,3-Dihydro-1′-[3-[(2S)-2-[[((3R)-1-methyl-3-pyrrolidinyl)amino]carbonyl]-2,3-dihydro-1H-indol-1-yl]-3-oxopropyl]spiro[1H-indene-1,4′-piperidine]citrate

To a stirred solution of 2,3-dihydro-1′-[3-[(2S)-2-[[((3R)-1H-3-pyrrolidinyl)amino]carbonyl]-2,3-dihydro-1H-indol-1-yl]-3-oxopropyl]spiro[1H-indene-1,4′-piperidine] (90 mg, 0.19 mmol, this was prepared in Example 42), 37% HCHO (77 μl, 0.95 mmol), and AcOH (33 μl, 0.57 mmol) in MeOH (4 ml) was added NaBH3CN (24 mg, 0.38 mmol) at room temperature. The resulting reaction mixture was stirred at room temperature for 16 h, then concentrated. The residue was quenched with aqueous NaHCO3 solution and extracted with CH2Cl2. The extracts combined were dried (MgSO4) and concentrated. The resulting residue was purified by preparative TLC (1 mm thick silica gel plate: CH2Cl2/MeOH/25% NH3:100/10/1) to afford 65 mg (71%) of free base as a colorless amorphous.

This compound showed broadened spectra in proton NMR.

This was converted to citric acid salt according to the procedure described in Example 34 to give 91 mg of the title product as a white amorphous solid.

MS (ESI positive) m/z: 487 (M+H)⁺.

IR(KBr): 3390, 2934, 1715, 1653, 1595, 1417, 1269, 760 cm⁻¹

Anal. Calcd for C30H38N4O2-C6H8O7-3.4H2O: C, 58.43; H, 7.19; N, 7.57. Found: C, 58.76; H, 7.05; N, 7.17.

### Preparation 17

### 2,3-Dihydro-1′-[3-[(2S)-2-[[((3S)-1-benzyl-3-pyrrolidinyl)amino]carbonyl]-2, dihydro-1H-indol-1-yl]-3-oxopropyl]spiro[1H-indene-1,4′-piperidine]

This was prepared according to the procedure described in Example 17 using (3S)-1-benzyl-3-aminopyrrolidine instead of N,N-dimethylethylenediamine. 375 mg (91%) of the title product was obtained as a pale yellow amorphous.

This compound showed broadened spectra in proton NMR.

MS (ESI positive) m/z: 563 (M+H)⁺.

### Example 44

### 2,3-Dihydro-1′-[3-[(2S)-2-[[((3S)-1H-3-pyrrolidinyl)amino]carbonyl]-2,3-dihydro-1H-indol-1-yl]-3-oxopropyl]spiro[1H-indene-1,4′-piperidine]citrate

This was prepared according to the procedure described in Example 42 using 2,3-dihydro-1′-[3-[(2S)-2-[[((3S)-1-benzyl-3-pyrrolidinyl)amino]carbonyl]-2,3-dihydro-1H-indol-1-yl]-3-oxopropyl]spiro[1H-indene-1,4′-piperidine] instead of 2,3dihydro-1′-[3-[(2S)-2-[[((3R)-1-benzyl-3-pyrrolidinyl)amino]carbonyl]-2,3-dihydro-1H-indol-1-yl]-3-oxopropyl]spiro[1H-indene-1,4′-piperidine]. 253 mg (82%) of free form of title compound was obtained as a pale yellow amorphous.

¹H NMR (270 MHz, CDCl3) δ 8.35-8.10 (0.3H, m), 7.40-6.60 (7.7H, m), 5.25-4.80 (1H, m), 4.45-4.25 (1H, m), 3.70-2.50 (16H, m), 2.35-2.20 (2H, m), 2.15-1.85 (3H, m), 2.01 (2H, t, J=7.3 Hz), 1.65-1.40 (3H, m).

This product (75 mg) was converted to citric acid salt according to the procedure described in Example 34 to give 105 mg of the title product as a white amorphous solid.

MS (ESI positive) m/z: 473 (M+H)⁺.

IR(KBr): 3416, 3020, 2939, 1719, 1663, 1578, 1483, 1414, 1269, 758 cm⁻¹

### Example 45

### 2,3-Dihydro-1′-[3-[(2S)-2-[[((3S)-1-methyl-3-pyrrolidinyl)amino]carbonyl]-2,3-dihydro-1H-indol-1-yl]-3-oxopropyl]spiro[1H-indene-1,4′-piperidine]citrate

This was prepared according to the procedure described in Example 43 using 2,3-dihydro-1′-[3-[(2S)-2-[[((3S)-1H-3-pyrrolidinyl)amino]carbonyl]-2,3-dihydro-1H-indol-1-yl]-3-oxopropyl]spiro[H-indene-1,4′-piperidine] (this was prepared described in Example 44) instead of 2,3-dihydro-1′-[3-[(2S)-2-[[((3R)-1H-3-pyrrolidinyl)amino]carbonyl]-2,3dihydro-1H-indol-1-yl]-3-oxopropyl]spiro[1H-indene-1,4′-piperidine]. 81 mg (75%) of free form of title compound was obtained as a colorless amorphous.

¹H NMR (270 MHz, CDCl3) δ 8.35-8.10 (0.3H, m), 7.30-6.40 (7.7H, m), 5.25-4.85 (1H, m), 4.504.33 (1H, m), 3.75-2.45 (13H, m), 2.40-2.10 (4H, m), 2.31 (3H, s), 2.08-1.85 (3H, m), 2.01 (2H, t, J=7.2 Hz), 1.65-1.40 (3H, m).

This was converted to citric acid salt according to the procedure described in Example 34 to give 108 mg of the title product as a white amorphous solid.

MS (ESI positive) m/z: 487 (M+H)⁺.

IR(KBr): 3422, 3042, 2939, 1719, 1663, 1597, 1483, 1414, 1269, 760 cm⁻¹

Anal. Calcd for C30H38N4O2-C6H8O7-2.8H2O: C, 59.30; H, 7.13; N, 7.68. Found: C, 59.55; H, 7.05; N, 7.23.

### Example 46

### 2,3-Dihydro-1′-[3-[(2S)-2-[(ethylamino)carbonyl]-2,3-dihydro-1H-indol-1-yl]-3-oxopropyl]spiro[1H-indene-1,4′-piperidine]citrate

This was prepared according to the procedure described in Example 17 using ethylamine instead of N,N-dimethylethylenediamine. 136 mg (98%) of free form of title compound was obtained as colorless amorphous.

¹H NMR (270 MHz, DMSO-d6) δ 8.38-8.28 (1H, m), 8.15-8.05 (1H, m), 7.24-7.10 (6H, m), 7.03-6.94 (1H, m), 5.05-4.95 (1H, m), 3.64-3.46 (1H, m), 3.20-2.60 (8H, m), 2.84 (2H, t, J=7.4 Hz), 2.45-2.05 (3H, m), 1.95 (2H, t, J=7.4 Hz), 1.88-1.75 (2H, m), 1.55-1.40 (2H, m), 1.04 (3H, t, J=7.3 Hz).

This was converted to citric acid salt according to the procedure described in Example 34 to give 186 mg of the title product as a white amorphous solid.

MS (ESI positive) m/z: 432 M+H)⁺.

Anal. Calcd for C27H33N3O2-C6H8O7-1.5H2O: C, 60.91; H, 6.82; N, 6.46. Found: C, 61.10; H, 6.80; N, 6.09.

### Example 47

### 2,3-Dihydro-1′-[3-[(2S)-2-[(cyclopropylamino)carbonyl]-2,3-dihydro-1H-indol-1 yl]-3-oxopropyl]spiro[1H-indene-1,4′-piperidine]citrate

This was prepared according to the procedure described in Example 17 using cyclopropylamine instead of N,N-dimethylethylenediamine. 109 mg (83%) of free form of title compound was obtained as colorless amorphous.

¹H NMR (300z, DMSO-d6) δ 8.46-8.39 (1H, m), 8.09 (1H, d, J=7.9 Hz), 7.22-7.08 (6H, m), 7.02-6.94 (1H, m), 4.994.89 (1H, m), 3.61-3.46 (1H, m), 3.03-2.55 (7H, m), 2.84 (2H, t, 3=7.3 Hz), 2.40-2.05 (3H, m), 1.96 (2H, t, J=7.3 Hz), 1.85-1.70 (2H, m), 1.50-1.38 (2H, m), 0.70-0.60 (2H, m), 0.48-0.40 (2H, m).

This was converted to citric acid salt according to the procedure described in Example 34 to give 132 mg of the title product as a white amorphous solid.

MS NISI positive) m/z: 444 M+H)⁺.

Anal. Calcd for C28H33N3O2-C6H8O7-2H2O: C, 60.79; H, 6.75; N, 6.26. Found: C, 60.96; H, 6.51; N, 6.87.

### Example 48

### 2,3-Dihydro-1′-[3-[(2S)-2-(1-piperidinylcarbonyl)-2,3-dihydro-1H-indol-1-yl]-3-oxopropyl]spiro[1H-indene-1,4′-piperidine]citrate

This was prepared according to the procedure described in Example 17 using piperidine instead of N,N-dimethylethylenediamine. 112 mg (80%) of free form of title compound was obtained as pale yellow amorphous.

¹H NMR (270 MHz, DMSO-d6) δ 8.11 (1H, d, J=8.1 Hz), 7.25-7.10 (6H, m), 7.05-6.94 (1H, m), 5.70-5.60 (1H, m), 3.76-3.18 (5H, m), 3.05-2.50 (6H, m), 2.84 (2H, t, J=7.4 Hz), 2.35-2.10 (3H, m), 1.95 (2H, t, J=7.4 Hz), 1.88-1.35 (10H, m).

This was converted to citric acid salt according to the procedure described in Example 34 to give 145 mg of the title product as a white amorphous solid.

MS NISI positive) m/z: 472 (M+H)⁺.

Anal. Calcd for C30H37N3O2-C6H8O7-2.3H2O: C, 61.32; H, 7.09; N, 5.96. Found: C, 61.39; H, 6.59; N, 5.56.

### Example 49

### 2,3-Dihydro-1′-[3-[(2S)-2-[[N-[2-(dimethylamino)ethyl]-N-methylamino]carbonyl]-2,3-dihydro-1H-indol-1-yl]-3-oxopropyl]spiro[1H-indene-1,4′-piperidine]citrate

This was prepared according to the procedure described in Example 17 using N,N,N′-trimethylethylenediamine instead of N,N-dimethylethylenediamine. 96 mg (80%) of free form of title compound was obtained as a pale yellow amorphous.

This compound showed broadened spectra in proton NMR.

This product (68 mg) was converted to citric acid salt according to the procedure described in Example 34 to give 90 mg of the title product as a white amorphous solid. MS (ESI positive) m/z: 489 (M+H)⁺.

Anal. Calcd for C30H40N4O2-C6H8O7-2.5H2O: C, 59.57; H, 7.36; N, 7.72. Found: C, 59.83; H, 7.27; N, 7.17.

### Example 50

### 2,3-Dihydro-1′-[3-oxo-3-(3-oxo-3,4-dihydro-1 (2R)-quinoxalinyl)propyl]spiro[1H-indene-1,4′-piperidine]citrate

This was prepared according to the procedure described in Example 1 using 3,4-dihydro-1H-quinoxalin-2-one (this was prepared according to known procedure: TenBrink Ruth E. et al, J. Med. Chem. 1994, 37, 758) instead of methyl indoline-2-carboxylate. 23 mg (13%) of free form of title compound was obtained as pale brown oil.

¹H NMR (300 MHz, CDCl3) δ 9.06 (1H, s), 7.26-7.07 (7H, m), 7.01-6.96 (1H, m), 4.52 (2H, s), 2.87 (2H, t, J=7.3 Hz), 2.86-2.74 (6H, m), 2.26-2.12 (2H, m), 1.97 (2H, t, J=7.3 Hz), 1.96-1.80 (2H, m), 1.56-1.46 (2H, m).

This was converted to citric acid salt according to the procedure described in Example 34 to give 29 mg of the title product as a pale brown solid.

MS (ESI positive) m/z: 390 (M+H)⁺.

IR(KBr): 3402, 2930, 1693, 1601, 1504, 1394, 1198, 760 cm⁻¹

Anal. Calcd for C24H27N3O2-C6H8O7-0.4CH2Cl2-2H2O: C, 56.03; H, 6.16; N, 6.45.

Found: C, 55.87; H, 5.81; N, 6.08.

### Preparation 18

### 1-Acryloyl-1′-benzyloxycarbonylspiro[indoline-3,4′-piperidine]

To a stirred solution of acryloyl chloride (0.24 g, 2.61 mmol) in CH2Cl2 (5 ml) was added a mixture of 1′-benzyloxycarbonylspiro[indoline-3,4′-piperidine] (0.70 g, 2.17 mmol, this was prepared according to known procedure: Maligres Peter E. et al, Tetrahedron 1997, 53, 10983) and triethylamine (0.60 ml, 4.34 mmol) in CH2Cl2 (4 ml) at 0° C. The resulting reaction mixture was stirred at the same temperature for 20 min., quenched with aqueous NaHCO3 solution and extracted with CH2Cl2. The extracts combined were washed with d-HCl, dried (MgSO4), filtered, and concentrated.

The resulting residue was purified by silica gel column chromatography (hexane/AcOEt: 1/1 as an eluent) to afford 0.47 g (58%) of title compound as pale yellow amorphous.

¹H NMR (270 MHz, CDCl3) δ 8.40-8.25 (1H, m), 7.40-6.95 (8H, m), 6.70-6.40 (2H, m), 5.82-5.73 (1H, m), 5.15 (2H, s), 4.28-4.10 (2H, m), 3.99 (2H, s), 3.03-2.82 (2H, m), 1.87-1.70 (2H, m), 1.68-1.53 (2H, m).

### Preparation 19

### 2,3-Dihydro-1′-[3-[1′-benzyloxycarbonylspiro[indoline-3,4′-piperidine]-1-yl]-3-oxopropyl]spiro[1H-indene-1,4′-piperidine]

A mixture of 1-acryloyl-1′-benzyloxycarbonylspiro[indoline-3,4′-piperidine] (0.47 g, 1.3 mmol), 2,3-dihydrospiro[1H-indene-1,4′-piperidine]hydrochloride (0.31 g, 1.4 mmol), and tiethylamine (0.23 ml, 1.6 mmol) in THF (8 ml) was stirred at 60° C. for 16 h. Then the reaction mixture was quenched with NaHCO3 solution, and extracted with CH2Cl2. The extracts combined were dried (MgSO4), filtered, and concentrated. The residue was purified by silica gel column chromatography (CH2Cl2/MeOH: 40/1 as eluent) to give 0.57 g (72%) of title compound as colorless amorphous.

¹H NMR (270 MHz, CDCl3) δ 8.24 (1H, d, J=8.1 Hz), 7.45-7.02 (12H, m), 5.18 (2H, s), 4.34-4.18 (2H, m), 3.96 (2H, s), 3.10-2.70 (8H, m), 2.91 (2H, t, J=7.3 Hz), 2.38-2.22 (2H, m), 2.03 (2H, t, J=7.3 Hz), 2.00-1.53 (8H, m).

### Example 51

### 2,3-Dihydro-1′-[3-[spiro[indoline-3,4′-piperidine]-1-yl]-3-oxopropyl]spiro[1H-indene-1,4′-piperidine]citrate

A mixture of 2,3-Dihydro-1′-[3-[1′-benzyloxycarbonylspiro[indoline-3,4′-piperidine]-1-yl]-3-oxopropyl]spiro[1H-indene-1,4′-piperidine] (0.57 g, 1.00 mmol) and 10% Pd—C (50 mg) in MeOH (8 ml) was stirred at room temperature under hydrogen atmosphere for 14 h. The reaction mixture was filtered through a pad of celite and the filtrate was concentrated in vacuo to give crude product (0.42 g, 98%) as a colorless amorphous. This resulting crude product (90 mg) was purified by preparative TLC (1 mm thick silica gel plate: CH2Cl2/MeOH/25% NH3:100/10/1) to afford 74 mg (81%) of free base as a colorless amorphous.

¹HNMR (270 MHz, CDCl3) δ 8.23 (1H, d, J=7.9 Hz), 7.28-7.14 (6H, m), 7.11-7.03 (1H, m), 3.96 (2H, s), 3.20-3.08 (2H, m), 3.02-2.68 (10H, m), 2.38-2.24 (2H, m), 2.08-1.80 (7H, m), 1.72-1.52 (4H, m).

This was converted to citric acid salt according to the procedure described in Example 34 to give 101 mg of the title product as a white amorphous solid.

MS (ESI positive) m/z: 430 (M+H)⁺.

IR(KBr): 3412, 2932, 1717, 1653, 1597, 1483, 1420, 1281, 760 cm⁻¹

Anal. Calcd for C28H35N3O—C6H8O7-2H2O: C, 62.09; H, 7.20; N, 6.39. Found: C, 62.17; H, 7.16; N, 6.09.

### Example 52

### 2,3-Dihydro-1′-[3-[1′-methylspiro[indoline-3,4′-piperidine]-1-yl]-3-oxopropyl]spiro[1H-indene-1,4′-piperidine]citrate

This was prepared according to the procedure described in Example 43 using 2,3-Dihydro-1′-[3-[spiro[indoline-3,4′-piperidine]-1-yl]-3-oxopropyl]spiro(1H-indene-1,4′-piperidine] (this was prepared in Example 52) instead of 2,3-dihydro-1′-[3-[(2S)-2-[[((3R)-1H-3-pyrrolidinyl)amino]carbonyl]-2,3-dihydro-1H-indol-1-yl]-3-oxopropyl]spiro[1H-indene-1,4′-piperidine]. 127 mg (97%) of free form of title compound was obtained as a colorless amorphous.

¹H NMR (270 MHz, CDCl3) δ 8.22 (1H, d, J=8.1 Hz), 7.28-7.14 (6H, m), 7.10-7.02 (1H, m), 3.90 (2H, s), 3.04-2.70 (10H, m), 2.38-1.90 (10H, m), 2.36 (3H, s), 1.76-1.52 (4H, m).

This was converted to citric acid salt according to the procedure described in Example 34 to give 174 mg of the title product as a white amorphous solid.

MS (ESI positive) m/z: 444 (M+H)⁺.

IR(KBr): 3412, 2932, 1717, 1655, 1597, 1483, 1420, 1273, 760 cm⁻¹

Anal. Calcd for C29H37N3O—C6H8O7-2.5H2O: C, 61.75; H, 7.40; N, 6.17. Found: C, 61.86; H, 7.14; N, 5.81.

### Example 53

### 2,3-Dihydro-1′-[3-[(2S)-2-[(4-methyl-1-piperadinyl)carbonyl]-2,3-dihydro-1H-indol-1-yl]-3-oxopropyl]spiro[1H-indene-1,4′-piperidine]citrate

A mixture of 2,3-dihydro-1′-[3-(2-(S)-carboxyindolin-1-yl)-3-oxopropyl]spiro[1H-indene-1,4′-piperidine] (35 mg, 0.088 mmol, this was prepared in Preparation 9), N-methylpiperadine (29 μl, 0.263 mmol), WSC (50 mg, 0.263 mmol), HOBt (36 mg, 0.263 mmol), and triethylamine (37 μl, 0.263 mmol) in CH2Cl2 (3 ml) was stirred at room temperature for 18 h. The reaction mixture was diluted with saturated aqueous NaHCO3 solution and extracted with CH2Cl2. The extracts combined were dried (Na2SO4), filtered, and concentrated. The residue was purified by NH-silica gel column chromatography (50 g, Hexane/Acetone: 3/1) to give 46 mg (63%) of free form of title compound as colorless oil.

Two isomers with a ratio of 1:1 were observed in CDCl3 solution.

¹H NMR (270 MHz, CDCl3) δ 8.30 (0.5H, d, J=8.2 Hz), 7.33-7.07(6H, m), 7.00 (0.5H, t, J=7.4 Hz), 5.48 (0.5H, d, J=9.7 Hz), 5.23 (0.5h, d, J=9.1 Hz), 3.80-3.40 (5H, m), 3.25-2.80 (7H, m, including 2H, t, J=7.4 Hz at 2.90 ppm), 2.60-2.15 (8H, m), 2.17 (3H, s), 2.07-1.85 (4H, m, including 2H, t, J=7.4 Hz at 2.02 ppm), 1.56 (2H, d, J=13.8 Hz).

This was converted to citric acid salt according to the procedure described in Example 34 to give 70 mg of title compound as white amorphous solid.

MS (ESI positive) m/z: 487 (M+H)⁺.

IR(KBr): 3371, 2939, 1720, 1661, 1597, 1483, 1418, 1219, 976, 760 cm⁻¹

Anal. Calcd for C30H38N4O2-2C6H8O7-4.5H2O: C, 52.99; H, 6.67; N, 5.89. Found: C, 53.00; H, 6.49; N, 6.10.

### Example 54

### 2,3-Dihydro-1′-[3-[(2S)-2-[[[2-(1-pyrrolidinyl)ethyl]amino]carbonyl]-2,3-dihydro 1H-indol-1-yl]-3-oxopropyl]spiro[1H-indene-1,4′-piperidine]citrate

This was prepared according to the procedure described in Example 17 using 1-(2-aminoethyl)pyrrolidine instead of N,N-dimethylethylenediamine. 25 mg (41%) of free form of title compound was obtained as colorless oil.

This compound showed broadened spectra in proton NMR except for the following peaks.

¹H NMR (300 MHz, CDCl3) δ 2.50-2.25 (2H, m), 2.15-1.95(4H, m, including 2H, t, J=7.4 Hz at 2.02 ppm), 1.81 (4H, m), 1.59 (2H, d, J=13.0 Hz).

This was converted to citric acid salt according to the procedure described in Example 34 to give 32 mg of title compound as white amorphous solid.

MS (ESI positive) m/z: 501 (M+H)⁺.

IR(KBr): 3400, 2939, 1728, 1655, 1597, 1483, 1411, 1215, 760 cm⁻¹

Anal. Calcd for C31H40N4O2-2C6H8O7-3H₂O:C, 55.00;H, 6.66;N, 5.97. Found: C, 55.38; H, 6.53; N, 6.20.

### Example 55

### 2,3-Dihydro-1-[3-[(2S)-2-[[[2-(4-morpholinyl)ethyl]amino]carbonyl]-2,3-dihydro 1H-indol-1-yl]-3-oxopropyl]spiro[1H-indene-1,4′-piperidine]citrate

This was prepared according to the procedure described in Example 17 using N-(2-aminoethyl)morpholine instead of N,N-dimethylethylenediamine. 54 mg (85%) of free form of title compound was obtained as oil.

¹H NMR (270 MHz, CDCl3) δ 8.24 (1H, m), 7.30-7.13 (6H, m), 7.07 (1H, t, J=7.6 Hz), 6.88 (1H, br.s), 5.03 (1H, m), 3.75-3.40 (6H, m), 3.40-3.15 (4H, m), 3.15-2.83 (8H, m, including 2H, t, J=7.4 Hz at 2.91 ppm), 2.50-2.20 (6H, m), 2.10-1.94 (4H, m, including 2H, t, J=7.3 Hz at 2.02 ppm), 1.58 (2H, d, J=13.4 Hz).

This was converted to citric acid salt according to the procedure described in Example 34 to give 80 mg of title compound as white amorphous solid.

MS (ESI positive) m/z: 517 (M+H)⁺.

IR(KBr): 3400, 2941, 1732, 1653, 1597, 1483, 1461, 1416, 1211, 758 cm⁻¹

Anal. Calcd for C31H40N4O3-2C6H8O7-3H₂O: C, 54.08; H, 6.54; N, 5.87. Found: C, 54.01; H, 6.43; N, 5.74.

### Example 56

### 2,3-Dihydro-1′-[3-[(2S)-2-[(3-dimethylamino-1-pyrrolidinyl)carbonyl]-2,3-dihydro-1H-indol-1-yl]-3-oxopropyl]spiro[1H-indene-1,4′-piperidine]citrate

This was prepared according to the procedure described in Example 17 using 3-(Dimethylamino)pyrrolidine instead of N,N-dimethylethylenediamine. 56 mg (90%) of free form of title compound was obtained as red oil. This compound showed broadened spectra in proton NMR.

This was converted to citric acid salt according to the procedure described in Example 34 to give 88 mg of title compound as white amorphous solid.

MS (ESI positive) m/z: 501 (M+H)⁺.

IR(KBr): 3396, 2941, 2581, 1724, 1655, 1597, 1483, 1411, 1200, 759 cm⁻¹

Anal. Calcd for C31H40N4O2-2C6H8O7-3H2O: C, 55.00; H, 6.66; N, 5.97. Found: C, 55.43; H, 6.33; N, 5.57.

### Example 57

### 2,3-Dihydro-1′-[3-[(2S)-2-[[(4-piperidinyl)amino]carbonyl]-2,3-dihydro-1H-indol-1-yl]-3-oxopropyl]spiro[1H-indene-1,4′-piperidine]citrate

A mixture of 2,3-dihydro-1′-[3-(2-(S)-carboxyindolin-1-yl)-3-oxopropyl]spiro[1H-indene-1,4′-piperidine] (130 mg, 0.321 mmol, this was prepared in Preparation 9), 4-Amino-1-benzyl-piperidine (0.197 ml, 0.964 mmol), WSC (123 mg, 0.643 mmol), HOBt (88 mg, 0.643 mmol), and triethylamine (134 μl, 0.964 mmol) in CH2Cl2 (5 ml) was stirred at room temperature for 2 days. The reaction mixture was diluted with saturated aqueous NaHCO3 solution and extracted with CH2Cl2. The extracts combined were dried (Na2SO4), filtered, and concentrated. The residue was purified by NH-silica gel column chromatography (100 g, Hexane/Acetone: 2/1 as eluent) to give 230 mg of amido product as white amorphous solid. This compound was used for the next step without further purification.

MS (EI direct) m/z: 576 (M)⁺

A suspension mixture of this amido (230 mg), 10% palladium on activated carbon (100 mg) and MeOH (5 ml) was stirred under hydrogen atmosphere at room temperature for 20 h. After the removal of the catalyst by filtration, the filtrate was concentrated. The resulting crude oil was purified by preparative TLC (1 mm thick plate, CH2Cl2/MeOH/Et3N: 100/10/1) and recristalization (CH2Cl2-Et2O) to give 98 mg (63%, 2 steps) as free form of title compound as oil.

This compound showed broadened spectra in proton NMk.

This was converted to citric acid salt according to the procedure described in Example 34 to give 95 mg of title compound as white amorphous solid.

MS (ESI positive) m/z: 487 (M+H)⁺.

IR(KBr): 3400, 2943, 1655, 1597, 1483, 1420, 1242, 1215, 760 cm⁻¹

Anal. Calcd for C30H38N4O2-C6H8O7-3.4H2O: C, 58.43; H, 7.19; N, 7.57. Found: C, 58.76; H, 7.15; N, 7.16.

### Example 58

### 2,3-Dihydro-1′-[3-[(2S)-2-[[(1-methyl-4-piperidinyl)amino]carbonyl]-2,3-dihydro 1H-indol-1-yl]-3-oxopropyl]spiro[1H-indene-1,4′-piperidine]citrate

A mixture of 2,3-Dihydro-1′-[3-[(2S)-2-[[(4-piperidinyl)amino]carbonyl]-2,3-dihydro-1H-indol-1-yl]-3-oxopropyl]spiro[1H-indene-1,4′-piperidine] (58 mg, 0.119 mmol, this was prepared in Example 57), 37% formaldehyde solution in water (45 μl, 0.594 mmol) and CH3CN (2 ml) was added NaBH3CN (11 mg, 0.178 mmol) at room temperature, and the resulting mixture was stirred at room temperature for further 20 h. The reaction mixture was diluted with saturated aqueous NaHCO3 solution and extracted with CH2Cl2. The extracts combined were dried (Na2SO4), filtered, and concentrated. The residue was purified by preparative TLC (1 mm thick plate, CH2Cl2/MeOH/Et3N: 100/10/1) to give 37 mg (63%) of free form of title compound as white solid.

¹H NMR (600 MHz, DMSO-d6) δ 8.27 (1H, d, J=7.5 Hz), 8.09 (1H, d, J=8.0 Hz), 7.20-7.10 (6H, m), 6.97 (1H, t, J=7.4 Hz), 4.98 (1H, d, J=10.8 Hz), 3.61-3.48 (2H, m), 2.97 (1H, d, J=15.1 Hz), 2.84 (2H, t, J=7.3 Hz), 2.77 (2H, d, J=5.4 Hz), 2.74-2.53 (5H, m), 2.35-2.24 (1H, m), 2.22-2.09 (5H, m, including 3H, s, at 2.13 ppm), 2.00-1.90 (4H, m, including 2H, d, J=7.2 Hz at 1.94 ppm), 1.78 (2H, t, J=12.1 Hz), 1.71 (2H, t, J=11.1 Hz), 1.50-1.40 (4H, m).

¹³C NMR (150 MHz, CDCl3) δ 29.3, 31.1, 31.4, 32.4, 34.1, 34.4, 36.4, 36.4, 45.7, 45.8, 45.8, 50.3, 50.5, 53.4, 53.8, 53.8, 60.5, 115.9, 122.2, 123.0, 124.3, 124.3, 126.2, 126.4, 127.0, 129.8, 142.6, 143.6, 151.1, 170.3, 170.3.

This was converted to citric acid salt according to the procedure described in Example 34 to give 27 mg of title compound as white amorphous solid.

MS (ESI positive) m/z: 501 (M+H)⁺.

IR(KBr): 3227, 3047, 2939, 2710, 1664, 1597, 1558, 1483, 1271, 1242, 1215, 760 cm⁻¹

Anal. Calcd for C31H40N4O2-C6H8O7-3H2O: C, 59.50; H, 7.29; N, 7.50. Found: C, 59.37; H, 7.29; N, 7.59.

### Example 59

### 2,3-Dihydro-1′-[3-[(2S)-2-[(1-pyrrolidinyl)carbonyl]-2,3-dihydro-1H-indol-1-yl]-3-oxopropyl]spiro[1H-indene-1,4′-piperidine]citrate

A mixture of 2,3-dihydro-1′-[3-(2-(S)-carboxyindolin-1-yl)-3-oxopropyl]spiro[1H-indene-1,4′-piperidine] (70 mg, 0.173 mmol, this was prepared in Preparation 9), pyrrolidine (43 μL 0.519 mmol), WSC (66 mg, 0.346 mmol), HOBt (47 mg, 0.346 mmol), and triethylamine (72 μl, 0.519 mmol) in CH2Cl2 (2 ml) was stirred at room temperature for 1 day. The reaction mixture was diluted with saturated aqueous NaHCO3 solution and extracted with CH2Cl2. The extracts combined were dried (Na2SO4), filtered, and concentrated. The residue was purified by preparative TLC (1 mm thick plate, CH2Cl2/MeOH: 10/1) to give 50 mg (63%) of free form of title compound as oil.

¹H NMR (270 MHz, DMSO-d6) δ 8.11 (1H, d, J=8.1 Hz), 7.25-7.07 (6H, m), 6.98 (1H, t, J=7.6 Hz), 5.42 (1H, d, J=8.2 Hz), 3.75-3.56 (2H, m), 3.56-3.25 (4H, m), 3.04 (1H, d, J=17.0 Hz), 2.84 (2H, t, J=7.3 Hz), 2.95-2.50 (4H, m), 2.30-2.05 (3H, m), 2.05-1.88 (4H, m, including 2H, t, J=7.1 Hz at 1.94 ppm), 1.88-1.70 (4H, m), 1.42 (2H, d, J=13.5 Hz).

This was converted to citric acid salt according to the procedure described in Example 34 to give 48 mg of title compound as white amorphous solid.

MS (ESI positive) rn/z: 458 (M+H)⁺.

IR(KBr): 3400, 2953, 2882, 2570, 1732, 1649, 1597, 1485, 1340, 1312, 1191, 758 cm⁻¹

Anal. Calcd for C29H35N3O2-C6H8O7-1.5H2O: C, 62.12; H, 6.85; N, 6.21. Found: C, 62.42; H, 6.72; N, 6.00.

### Example 60

### 2,3-Dihydro-1′-[3-[(2S)-2-[(3-hydroxy-1-pyrrolidinyl)carbonyl]-2,3-dihydro-1H-indol-1-yl]-3-oxopropyl]spiro[1H-indene-1,4′-piperidine]citrate

A mixture of 2,3-dihydro-1′-[3-(2-(S)-carboxyindolin-1-yl)-3-oxopropyl]spiro[1H-indene-1,4′-piperidine] (100 mg, 0.247 mmol, this was prepared in Preparation 9), DL-3-pyrrolidinol (62 μl, 0.742 mmol), WSC (95 mg, 0.494 mmol), HOBt (67 mg, 0.494 mmol), and triethylamine (103 μl, 0.742 mmol) in CH2Cl2 (4 ml) was stirred at room temperature for 20 h. The reaction mixture was diluted with saturated aqueous NaHCO3 solution and extracted with CH2Cl2. The extracts combined were dried (Na2SO4), filtered, and concentrated. The residue was purified by silica gel column chlomatography (EtOAc/iPrOH/NH4OH: 100/20/1) to give 30 mg (25%) of free form of title compound as colorless oil.

This compound showed broadened spectra in proton NMR.

This was converted to citric acid salt according to the procedure described in Example 34 to give 16 mg of title compound as white amorphous solid.

MS (ESI positive) m/z: 474 (M+H)⁺.

IR(KBr): 3408, 2941, 1719, 1638, 1483, 1420, 1312, 1220, 1192, 760 cm⁻¹

Anal. Calcd for C29H35N3O3-C6H8O7-2.5H2O: C, 59.14; H, 6.81; N, 5.91. Found: C, 59.28; H, 6.77; N, 5.83.

### Preparation 20

### N-(tert-butoxycarbonyl)-2-{[(2-amino-2-oxoethyl)oxy]methyl}-2,3-dihydro-1H-indole

To a stirred solution of NaH (27 mg, 0.665 mmol, 60% oil dispersion in mineral oil) and N-(tert-butoxycarbonyl)-2-hydroxymethy-2,3-dihydro-1H-indole (138 mg, 0.554 mmol, this was prepared according to known procedure: Fujita, Takeshi et al, Eur. Pat. Appl. 1995, EP 676398) in DMF(3 ml) was added a solution of 2-bromoacetamide (153 mg, 8.94 mmol) in DMF (2 ml) at 0° C. The reaction mixture was stirred at room temperature for 20 h. Then the reaction mixture was heated to 100° C. with stirring for 2 days. The reaction mixture was cooled to room temperature, and quenched with water. The mixture was concentrated, diluted with EtOAc-toluene (½), and washed with water (twice) and brine. The organic layer was dried (Na2SO4), filtered, and concentrated. The residue was purified by silica gel column chromatography (Hexane/Acetone: 3/1 as eluent) to give 10 mg (6%) of title compound as colorless oil.

¹H NMR (300 MHz, CDCl3) δ 7.60 (1H, m), 7.20-7.12 (2H, m), 6.95 (1H, t, 3=7.3 Hz), 6.21 (1H, br. s), 5.42 (1H, br. s), 4.63 (1H, m), 3.92 (2H, d, J=2.4 Hz), 3.66 (2H, d, J=4.8 Hz), 3.34 (1H, dd, J=10.3 Hz, 16.3 Hz), 2.93 (1H, d, J=16.7 Hz), 1.58 (9H, s).

### Preparation 21

### 2-{((2-amino-2-oxoethyl)oxy]methyl}-2,3-dihydro-1H-indole

A mixture of N-(tert-butoxycarbonyl)-2-{[(2-amino-2-oxoethyl)oxy]methyl}-2,3-dihydro-1H-indole (11.6 mg, 0.0379 mmol, this was prepared in Preparation 20) and CH2Cl2 (2 ml) was added trifluoroacetic acid (1 ml) at 0° C. The resulting mixture was stirred at room temperature for 1 h. The reaction mixture was concentrated, besified with NaHCO3 solution, and extracted with CH2Cl2. The extracts combined were dried (Na2SO4), filtered, and concentrated. The residue was purified by preparative TLC (0.5 mm thick plate, Hexane/Acetone: 1/1) to give 7.0 mg (90%) of title compound as white amorphous solid.

¹H NMR (300 MHz, CDCl3) δ 7.09 (1H, d, J=7.3 Hz), 7.04 (1H, t, J=7.7 Hz), 6.75 (1H, br. s), 6.73 (1H, dt, J=0.9 Hz, 7.3 Hz), 6.65 (1H, d, J=8.1 Hz), 5.74 (1H, br. s), 4.174.06 (1H, m), 4.01 (1H, s), 4.00 (1H, s), 3.65-3.52 (2H, m), 3.17 (1H, dd, J=9.2 Hz, 15.8 Hz), 2.74 (1H, dd, J=7.2 Hz, 15.8 Hz), 1.71 (1H, br. s).

### Example 61

### 2,3-Dihydro-1′-[{3-[2-((2-amino-2-oxoethyl)oxy)methyl-2,3-dihydro-1H-indol-1-yl]-3-oxopropyl]spiro [H-indene-1,4′-piperidine]citrate

To a stirred solution of 2-{[(2-amino-2-oxoethyl)oxy]methyl}-2,3-dihydro-1H-indole (7.0 mg, 0.0339 mmol, this was prepared in Preparation 21) and triethylamine (14.2 μl, 0.1018 mmol) in CH2Cl2 (1 ml) was added 2,3-dihydro-1′-[2-(chloroformyl)ethyl]spiro[1H-indene-1,4′-piperidine]hydrochloride (11.7 mg, 0.0373 mmol, this was prepared in Preparation 3) at 0° C. and the resulting reaction mixture was stirred at room temperature for 20 h. The reaction mixture was poured into a saturated aqueous NaHCO3 solution and extracted with EtOAc. The organic layer was washed with saturated aqueous NaHCO3 solution and brine, dried (Na2SO4), filtered, and concentrated. The residue was purified by preparative TLC (0.5 mm thick plate, CH2Cl2/MeOH: 10/1) to give 12.4 mg (82%) of free form of title compound as white amorphous solid.

This compound showed broadened spectra in proton NMR except for the following peaks.

¹H NMR (300 MHz, CDCl3) δ 2.35 (2H, m), 2.07-1.92 (4H, m, including 2H, t, J=7.3 Hz at 2.03 ppm), 1.59 (2H, d, J=13.2 Hz).

This was converted to citric acid salt according to the procedure described in Example 34 to give 16.2 mg of title compound as white amorphous solid.

MS (ESI positive) m/z: 448 (M+H)⁺.

### Example 62

### 2,3-Dihydro-1′-[3-oxo-3-(2,3,4,5-tetrahydro-1H-benzazepin-1-yl)propyl]spiro[1H-indene-1,4′-piperidine]citrate

To a stirred solution of 2,3,4,5-tetrahydro-1H-benzazepine (74 mg, 0.501 mmol, this was prepared according to known procedure:B. D. Astill et al, J. Amer. Chem. Soc. 1955, 77, 4079) and triethylamine (0.21 ml, 1.504 mmol) in CH2Cl2 (5 ml) was added 2,3-dihydro-1′-[2-(chloroformyl)ethyl]spiro[1H-indene 1,4′-piperidine]hydrochloride (0.173 g, 0.551 mmol, this was prepared in Preparation 3) at 0° C. and the resulting reaction mixture was stirred at room temperature for 20 h. The reaction mixture was poured into a saturated aqueous NaHCO3 solution and extracted with EtOAc. The organic layer was washed with saturated aqueous NaRCO3 solution and brine, dried (Na2SO4), filtered, and concentrated. The residue was purified by silica gel column chromatography (Hexane/Acetone: 3/1-1/1 as eluent) to give 93 mg (48%) of free form of title compound as colorless oil.

¹H NMR (270 MHz, CDCl3) δ 7.27-7.10 (8H, m), 4.72 (1H, br. d, J=14.2 Hz), 2.86 (2H, t, J=7.3 Hz), 2.80-2.55 (6H, m), 2.52-2.38 (1H, m), 2.28-1.70 (11H, m, including 2H, t, J=7.3 Hz at 1.95 ppm), 1.55-1.30 (3H, m, including 2H, d, J=13.4 Hz at 1.47 ppm).

MS (EI direct) m/z: 388 (M)⁺.

This was converted to citric acid salt according to the procedure described in Example 34 to give 78 mg of title compound as white amorphous solid.

MS (ESI positive) m/z: 389 (M+H)⁺.

IR(KBr): 2937, 2567, 1724, 1645, 1443, 1420, 1211, 764 cm⁻¹

Anal. Calcd for C26H32N2O—C6H8O7-1.5H2O: C, 63.25; H, 7.13; N, 4.61. Found: C, 63.51; H, 7.07; N, 4.42.

### Example 63

### 2,3-Dihydro-1′-[3-[(2S)-2-[(3-amino-1-pyrrolidinyl)carbonyl]-2,3-dihydro-1H-indol-1-yl]-3-oxopropyl]spiro[1H-indene-1,4′-piperidine]citrate

A mixture of 2,3-dihydro-11′-[3-(2-(S)-carboxyindolin-1-yl)-3-oxopropyl]spiro[1H-indene-1,4′-piperidine] (0.200 g, 0.494 mmol, this was prepared in Preparation 9), 3-(Boc-amino)pyrrolidine (0.276 g, 1.483 mmol), WSC (0.190 g, 0.989 mmol), HOBt (0.135 g, 0.989 mmol), and triethylamine (0.207 ml, 1.483 mmol) in CH2Cl2 (10 ml) was stirred at room temperature for 20 h. The reaction mixture was diluted with saturated aqueous NaHCO3 solution and extracted with CH2Cl2. The extracts combined were dried (Na2SO4), filtered, and concentrated. The residue was purified by silica gel column chromatography (50 g, CH2Cl2/MeOH: 10/1 as eluent) to give 0.283 g (99%) of amido product as yellow oil. This compound was used for the next step without further purification.

A mixture of this amido (0.283 g, 0.494 mmol), and CH2Cl2 (4 ml) was added trifluoroacetic acid (2 ml) at 0° C. The resulting mixture was stirred at room temperature for 1 h. The reaction mixture was concentrated, besified with NaHCO3 solution, and extracted with CH2Cl2. The extracts combined were dried (Na2SO4), filtered, and concentrated. The residue was purified NH-silica gel column chlomatography (50 g, Hexane/Acetone: 1/1 as eluent) to give 0.170 g (73%) of free form of title compound as an oil.

This compound showed broadened spectra in proton NMR.

This was converted to citric acid salt according to the procedure described in Example 34 to give 0.154 g of title compound as white amorphous solid.

MS (ESI positive) m/z: 473 (M+H)⁺.

IR(KBr): 3400, 2937, 1649, 1597, 1483, 1404, 1267, 1213, 760 cm⁻¹

Anal. Calcd for C29H36N4O2-C6H8O7-2.4H2O: C, 59.38; H, 6.95; N, 7.91. Found: C, 59.78; H, 6.89; N, 7.46.

### Example 64

### 2,3-Dihydro-1′-[3-[(2S)-2-[(1-azetidinyl)carbonyl]-2,3-dihydro-1H-indole-1-yl]-3-oxopropyl]spiro[1H-indene-1,4′-piperidine]citrate

A mixture of 2,3-dihydro-1′-[3-(2-(S)-carboxyindolin-1-yl)-3-oxopropyl]spiro[1H-indene-1,4′-piperidine] (120 mg, 0.297 mmol, this was prepared in Preparation 9), azetidine hydrochloride (56 mg 0.593 mmol), WSC (114 mg, 0.593 mmol), HOBt (81 mg, 0.593 mmol), and triethylamine (0.124 ml, 0.890 mmol) in CH2Cl2 (5 ml) was stirred at room temperature for 1 day. The reaction mixture was diluted with saturated aqueous NaHCO3 solution and extracted with CH2Cl2. The extracts combined were dried (Na2SO4), filtered, and concentrated. The residue was purified by preparative TLC (1 mm thick plate, CH2Cl2/MeOH: 10/1) to give 107 mg (81%) of free form of title compound as oil.

¹H NMR (270 MHz, DMSO-d6) δ 8.09 (1H, d, J=7.8 Hz), 7.25-7.10 (6H, m), 6.99 (1H, t, J=7.3 Hz), 5.20 (1H, d, J=8.7 Hz), 4.35-4.15 (2H, m), 3.92 (2H, m), 3.57 (1H, dd, J=11.5 Hz, 16.2 Hz), 2.95-2.78 (4H, m, including 2H, t, J=7.1 Hz at 2.84 ppm), 2.78-2.60 (2H, m), 2.36-2.10 (4H, m), 1.96 (2H, t, J=7.4 Hz), 1.81 (1H, br. t, J=12.4 Hz), 1.45 (2H, d, J=13.0 Hz).

This was converted to citric acid salt according to the procedure described in Example 34 to give 118 mg of title compound as white amorphous solid.

MS (ESI positive) m/z: 444 (M+H)⁺.

IR(KBr): 3414, 2943, 2571, 1728, 1653, 1483, 1418, 1217, 760 cm⁻¹

Anal. Calcd for C28H33N3O2-C6H8O7-1.8H2O: C, 61.12; H, 6.73; N, 6.29. Found: C, 61.04; H, 6.67; N, 6.08.

### Preparation 22

### (2S)-1-acryloyl-N,N-dimethyl-2,3-dihydro-1H-indole-2-carboxamide

To a stirred solution of (2S)-N,N-dimethyl-2,3-dihydro-1H-indole-2-carboxamide (11.07 g, 0.0511 mol, this was prepared according to known procedure:Serradeil-le Gal et al. PCT Int. Appl. 2001, WO 0164668) and triethylamine (17.81 ml, 0.1278 mol) in CH2Cl2 (200 ml) was added Acryloyl chloride (4.98 ml, 0.0613 mol) at 0° C. and the resulting reaction mixture was stirred at 0° C. for 2 h. The reaction mixture was poured into a saturated aqueous NaHCO3 solution and extracted with CH2Cl2. The extracts combined were dried (NaSO4), filtered, and concentrated. The residue was purified by silica gel column chromatography (500 g, Hexane/Acetone: 2/1-1/1 as eluent) to give 8.00 g (64%) of title compound as white solid.

This compound showed broadened spectra in proton NMR.

MS (EI direct) m/z: 244 (M)⁺.

### Example 65

### 1′-[3-[(2S)-2-[(dimethylamino)carbonyl]-2,3-dihydro-1H-indol-1-yl]-3-oxopropyl]spiro[1H-indene-1,4′-piperidine]citrate

A mixture of (2S)-1-acryloyl-N,N-dimethyl-2,3-dihydro-1H-indole-2-carboxamide (66 mg 0.271 mmol, this was prepared in Preparation 22), Spiro[1H-indene-1,4′-piperidine]hydrochloride (120 mg, 0.226 mmol), and triethylamine (94 μl, 0.677 mmol) in THF (3 ml) was stirred at 60° C. for 1 day. The reaction mixture was cooled to room temperature and evapolated to remove the solvent. The residue was purified silica gel column chromatography (50 g, Hexane/Acetone: 3/2 then CH2Cl2/MeOH: 10/1 as eluent) to give 90 mg (93%) of free form of title compound as oil. Two isomers with a ratio of 1:1 were observed in CDCl3 solution.

¹H NMR (270 MHz, CDCl3) δ 8.31 (0.5H, d, J=7.9 Hz), 7.42-7.07 (6.5H, m), 7.00 (1H, t, J=7.4 Hz), 6.85 (1H, d, 3=5.6 Hz), 6.75 (1H, d, J=5.6 Hz), 5.47 (0.5H, br. d, J=7.6 Hz), 5.26 (0.5H, br. d, J=7.9 Hz), 3.69 (0.5H, dd, J=11.4 Hz, 15.2 Hz), 3.46 (0.5H, dd, J=11.2 Hz, 16.0 Hz), 3.25-2.90 (12H, m), 2.70-2.36 (3H, m), 2.22 (2H, dt, J=3.5 Hz, 13.0 Hz), 1.38 (2H, d, J=13.4 Hz).

This was converted to citric acid salt according to the procedure described in Example 34 to give 106 mg of title compound as white amorphous solid.

MS (ESI positive) m/z: 430 (M+H)⁺.

IR(KBr): 3420, 2937, 2580, 1728, 1651, 1485, 1404, 1269, 1186, 754 cm⁻¹

Anal. Calcd for C27H31N3O2-C6H8O7-2H2O: C, 60.26; H, 6.59; N, 6.39. Found: C, 60.01; H, 6.36; N, 5.99.

### Example 66

### 1′-[3-[(2S)-2-[(dimethylamino)carbonyl]-2,3-dihydro-1H-indol-1-yl]-3-oxopropyl]spiro[isobenzofuran-[(3R),4′-piperidin]-3-one citrate

A mixture of (2S)-1-acryloyl-N,N-dimethyl-2,3-dihydro-1H-indole-2-carboxamide (72 mg 0.295 mmol, this was prepared in Preparation 17), Spiro[isobenzofuran-[(3B),4′-piperidin]-3-one hydrochloride (50 mg, 0.246 mmol), and triethylamine (51 μl, 0.369 mmol) in THF (3 ml) was stirred at 60° C. for 1 day. The reaction mixture was cooled to room temperature and evapolated to remove the solvent. The residue was purified silica gel column chromatography (50 g, Hexane/Acetone: 3/2 then CH2Cl2/MeOH: 10/1 as eluent) to give 103 mg (94%) of free form of title compound as oil.

Two isomers with a ratio of 1:1 were observed in CDCl3 solution.

¹H NMR (270 MHz, CDCl3) δ 8.31 (0.5H, d, J=7.6 Hz), 7.88 (1H, d, J=7.6 Hz),7.67 (1H, t, J=7.4 Hz), 7.52 (1H, t, J=7.6 Hz), 7.42 (1H, d, J=7.6 Hz), 7.32-7.07 (2.5H, m), 7.00 (1H, t, J=7.1 Hz), 5.48 (0.5H, br. d, J=7.8 Hz), 5.24 (0.5H, br. d, J=11.0 Hz), 3.71 (0.5H, dd, J=11.9 Hz, 14.7 Hz), 3.48 (0.5H, dd, J=10.9 Hz, 15.8 Hz), 3.25-2.85 (11H, m), 2.66 (2H, br. t, J=12.2 Hz), 2.60-2.38 (1H, m), 2.38-2.15 (3H, m), 1.74 (2H, d, J=13.2 Hz).

This was converted to citric acid salt according to the procedure described in Example 34 to give 120 mg of title compound as white amorphous solid.

MS (ESI positive) m/z: 448 (M+H)⁺.

IR(KBr): 3420, 2936, 2571, 1767, 1734, 1653, 1485, 1406, 1200, 1059, 932, 760 cm⁻¹

Anal. Calcd for C26H29N3O4-C6H8O7-2.5H2O: C, 56.13; H, 6.18; N, 6.14. Found: C, 56.14; H, 5.89; N, 5.79.

### Example 67

### 1′-[3-[(2S)-2-[(dimethylamino)carbonyl]-2,3-dihydro-1H-indol-1-yl]-3-oxopropyl]spiro[benzofuran-3(2B),4′-piperidin]-2-one citrate

A mixture of (2S)-1-acryloyl N,N-dimethyl-2,3-dihydro-1H-indole-2-carboxamide (72 mg 0.295 mmol, this was prepared in Preparation 17), Spiro[benzofuran-3(2H),4′-piperidin]-2-one hydrochloride (50 mg, 0.246 mmol), and triethylamine (51 μl, 0.369 mmol) in THF (3 ml) was stirred at 60° C. for 1 day. The reaction mixture was cooled to room temperature and evapolated to remove the solvent. The residue was purified silica gel column chromatography (50 g, Hexane/Acetone: 3/2 then CH2Cl2/MeOH: 1.0/1 as eluent) to give 22 mg (20%) of free form of title compound as colorless oil. Two isomers with a ratio of 1:1 were observed in CDCl3 solution.

¹H NMR (270 MHz, CDCl3) δ 8.30 (0.5H, d, J=7.9 Hz), 7.40-7.07 (6.5H, m), 7.00 (1H, t, J=7.9 Hz), 5.48 (0.5H, br. d, J=7.4 Hz), 5.31 (0.5H, br. d, J=6.3 Hz), 3.72 (0.5H, dd, J=10.9 Hz, 15.7 Hz), 3.47 (0.5H, dd, J=10.9 Hz, 15.8 Hz), 3.25-2.70 (14H, m), 2.63-2.35 (1H, m), 2.10-1.94 (4H, m).

This was converted to citric acid salt according to the procedure described in Example 34 to give 120 mg of title compound as white amorphous solid.

MS (ESI positive) m/z: 448 (M+H)⁻.

IR(KBr): 3422, 2937, 2588, 1793, 1732, 1653, 1485, 1406, 1230, 1055, 758 cm⁻¹

Anal. Calcd for C26H29N3O4-C6H8O7-3H2O: C, 55.41; H, 6.25; N, 6.06. Found: C, 55.71; H, 5.89; N, 5.56.

### Example 68

### 1′-[3-[(2S)-2-[(dimethylamino)carbonyl]-2,3-dihydro-1H-indol-1-yl]-3-oxopropyl]spiro[isobenzofuran-[(3R),4′-piperidine]citrate

A mixture of (2S)-1-acryloyl-N,N-dimethyl-2,3-dihydro-1H-indole-2-carboxamide (77 mg 0.317 mmol, this was prepared in Preparation 17), Spiro[isobenzofuran-[(3H),4′ piperidine]hydrochloride (50 mg, 0.264 mmol), and triethylamine (55 μl, 0.396 mmol) in THF (3 ml) was stirred at 60° C. for 1 day. The reaction mixture was cooled to room temperature and evapolated to remove the solvent. The residue was purified silica gel column chromatography (50 g, Hexane/Acetone: 3/2 then CH2Cl2/MeOH: 10/1 as eluent) to give 107 mg (94%) of free form of title compound as an oil.

Two isomers with a ratio of 1:1 were observed in CDCl3 solution.

¹H NMR (270 MHz, CDCl3) δ 8.30 (0.5H, d, J=8.1 Hz), 7.32-7.07 (6.5H, m), 6.99 (1H, t, J=7.4 Hz), 5.47 (0.5H, br. d, J=7.9 Hz), 5.26 (0.5H, br. d, J=8.7 Hz), 5.07 (2H, s), 3.69 (0.5H, dd, J=12.8 Hz, 13.8 Hz), 3.45 (0.5H, dd, J=11.5 Hz, 15.3 Hz), 3.22-2.85 (12H, m), 2.70-2.42 (3H, m), 2.03 (2H, dt, J=4.3 Hz, 13.2 Hz), 1.79 (2H, d, J=12.9 Hz).

This was converted to citric acid salt according to the procedure described in Example 34 to give 130 mg of title compound as white amorphous solid.

MS (ESI positive) m/z: 434 (M+H)⁺.

IR(KBr): 3435, 2934, 2573, 1732, 1655, 1485, 1418, 1045, 1020, 758 cm⁻¹

Anal. Calcd for C26H31N3O3-C6H8O7-2H2O: C, 58.09; H, 6.55; N, 6.35. Found: C, 57.85; H, 6.46; N, 6.08.

### Example 69

### 1′-[3-(2S)-2-[(dimethylamino)carbonyl]-2,3-dihydro-1H-indol-1-yl]-3-oxopropyl]spiro[benzofuran-3(2H),4′-piperidine]citrate

A mixture of (2)-1-acryloyl-N,N-dimethyl-2,3-dihydro-1H-indole-2-carboxamide (89 mg 0.365 mmol, this was prepared in Preparation 17), Spiro[benzofuran-3(2H),4′-piperidine] (62 mg, 0.304 mmol), and triethylamine (85 μl, 0.609 mmol) in THF (3 ml) was stirred at reflux temperature for 20 h. The reaction mixture was cooled to room temperature and evapolated to remove the solvent. The residue was purified silica gel column chromatography (50 g, EtOAc/rOH/25% NH4OH: 100/20/1 then CH2Cl2/MeOH: 10/1 as eluent) to give 91 mg (69%) of free form of title compound as oil.

¹H NMR (300 MHz, DMSO-d6) δ 8.11 (1H, d, J=8.1 Hz), 7.25-7.15 (3H, m), 7.10, 1H, dt, J=1.5 Hz, 7.9 Hz), 6.97 (1H, t, J=8.1 Hz), 6.84 (1H, t, J=7.3 Hz), 6.75 (1H, d, J=7.9 Hz), 5.61 (1H, dd, J=2.8 Hz, 11.0 Hz), 4.35 (2H, s), 3.64 (1H, dd, J=11.2 Hz, 16.7 Hz), 3.12 (3H, s), 3.01 (1H, dd, J=16.7 Hz), 2.88 (3H, s), 2.88-2.75 (2H, m), 2.75-2.55 (3H, m), 2.21-1.95 (3H, m), 1.84 (2H, br. t, J=11.7 Hz), 1.61 (2H, d, J=13.0 Hz).

This was converted to citric acid salt according to the procedure described in Example 34 to give 98 mg of title compound as white amorphous solid.

MS (ESI positive) m/z: 434 (M+H)⁺.

IR(KBr): 3422, 2936, 2573, 1719, 1653, 1483, 1406, 974, 756 cm⁻¹

Anal. Calcd for C26H31N3O3-C6H8O7-3H2O: C, 58.89; H, 6.49; N, 6.44. Found: C, 58.72; H, 6.37; N, 6.27.

### Preparation 23

### Spiro[(2-indanone)-1,4′-piperidine]

To a stirred solution of N-tert-butoxycarbonylspiro[(2-indanone)-1,4′-piperidine] (198 mg, 0.658 mmol, this was prepared according to known procedure:Toshiyasu Takemoto et al. Tetrahedron Asymmetry 1999, 10, 1787) in CH2Cl2 (2 ml) was added trifluoroacetic acid (1 ml) at room temperature and the resulting reaction mixture was stirred for 2 h. The reaction mixture was evapolated to remove the solvents, poured into a saturated aqueous NaHCO3 solution and extracted with CH2Cl2. The extracts combined were dried (Na2SO4), filtered, and concentrated to give 88 mg (67%) of title compound as brown oil.

¹H NMR (300 MHz, CDCl3) δ 7.40-7.23 (4H, m), 3.58 (2H, s), 3.35-3.20 (2H, m), 3.10-2.95 (2H, m), 2.44 (1H, br. s), 1.85-1.73 (4H, m).

### Example 70

### 1′-[3-[(2S)-2-[(dimethylamino)carbonyl]-2,3-dihydro-1H-indol-1-yl]-3-oxopropyl]spiro[(2-indanone)-1,41-piperidine]citrate

A mixture of (2S)-1-acryloyl-N,N-dimethyl-2,3-dihydro-1H-indole-2-carboxamide (0.193 g, 0.789 mmol, this was prepared in Preparation 22), 1-[4-Spiro-piperidine]-2-indanone (88 mg, 0.439 mmol, this was prepared in Preparation 23), and triethylamine (0.183 ml, 1.315 mmol) in THF (4 ml) was stirred at reflux temperature for 1 day. The reaction mixture was cooled to room temperature and evapolated to remove the solvent. The residue was purified silica gel column chromatography (50 g, CH2Cl2/MeOH: 15/1 as eluent) to give 81 mg (41%) of free form of title compound as oil.

Two isomers with a ratio of 1:1 were observed in CDCl3 solution.

¹H NMR (270 MHz, CDCl3) δ 8.31 (0.5H, d, J=7.9 Hz), 7.42-7.07 (6.5H, m), 7.00 (1H, t, J=7.4 Hz), 6.85 (1H, d, J=5.6 Hz), 6.75 (1H, d, J=5.6 Hz), 5.47 (0.5H, br. d, J=7.6 Hz), 5.26 (0.5H, br. d, J=7.9 Hz), 3.69 (0.5H, dd, J=11.4 Hz, 15.2 Hz), 3.46 (0.5H, dd, J=11.2 Hz, 16.0 Hz), 3.25-2.90 (12H, m), 2.70-2.36 (3H, m), 2.22 (2H, dt, J=3.5 Hz, 13.0 Hz), 1.38 (2H, d, J=13.4 Hz).

This compound (25 mg) was converted to citric acid salt according to the procedure described in Example 34 to give 28 mg of title compound as white amorphous solid.

MS (ESI positive) m/z: 446 (M+H)⁺.

### Example 71

### 1′-[3-[(2S)-2-[(dimethylamino)carbonyl]-2,3-dihydro-1H-indol-1-yl]-3-oxopropyl]spiro[(2-hydroxy)indane-1,4′-piperidine]citrate

To a stirred solution of 1′-[3-[(2S)-2-[(dimethylamino)carbonyl]-2,3-dihydro-1H-indol-1-yl]3-oxopropyl]spiro[3-(2-indanone)-1,4′-piperidine] (40 mg, 0.090 mmol, this was prepared in Example 70) in MeOH (1 ml) was added NaBH4 (4.1 mg, 1.077 mmol) at 0° C., and the resulting mixture was stirred for 2 h. The reaction mixture was quenched with water, diluted with a saturated aqueous NaHCO3 solution and extracted with CH2Cl2. The extracts combined were dried (Na2SO4), filtered, and concentrated. The residue was purified by preparative TLC (0.5 mm thick plate, CH2Cl2/MeOH/25% NH4OH: 100/10/1) to give 23 mg (58%) of free form of title compound as yellow solid. This compound showed broadened spectra in proton NMR except for the following peaks.

¹H NMR (300 MHz, CDCl3) δ 2.65-2.40 (3H, m), 2.40-2.07 (4H, m), 2.07-1.90 (1H, m), 1.76 (1H, br. t, J=10.3 Hz), 1.55 (1H, d, J=14.1 Hz).

This was converted to citric acid salt according to the procedure described in Example 34 to give 28 mg of title compound as white amorphous solid.

MS (FAB positive) m/z: 448 (M+H)⁺.

### Preparation 24

### N-tert-butoxycarbonylspiro[(2-hydroxy-3-methyl)indane-1,4′-piperidine]

To a stirred suspension of CuI (101 mg, 0.531 mmol) in THF (30 ml) was added slowly MeMgI (15.8 ml, 0.0133 mol, 0.84 mol/l in Et2O) at −20° C. under N2. After 10 minutes, N-tert-butoxycarbonylspiro[((2,3)-epoxy)indan-1,4′-piperidine] (800 mg, 2.65 mmol, this was prepared according to known procedure: Toshiyasu Takemoto et al. Tetrahedron Asymmetry 1999, 10, 1787) in THF (10 ml) was added dropwise. The resulting reaction mixture was stirred at −20° C. for 2 h. Excess of reagent was destroyed with saturated aqueous NH4Cl solution, besified with saturated aqueous NaHCO3 solution and extracted with EtOAc. The extracts combined were washed with water and brine, dried (Na2SO4), filtered, and concentrated. The residue was purified by silica gel column chromatography (200 g, Hexane/EtOAc: 3/1 as eluent) to give 0.372 g (44%) of title compound as an oil.

¹H NMR (300 MHz, CDCl3) δ 7.40-7.20 (4H, m), 3.85-3.67 (4H, m), 3.51-3.40 (1H, m), 3.11-3.00 81H, m), 2.07-1.96 (2H, m), 1.90-1.75 (2H, m), 1.49 (9H, s), 1.40 (3H, d, J=6.8 Hz).

### MS (EI direct) m/z: 317 (M)⁺

### Preparation 25

### N-tert-Butoxycarbonylspiro[[(2-(methylthiocarbonothioyl)oxy)-3-methyl}indane-1,4′-piperidine]

To a stirred solution of N-tert-Butoxycarbonyl-spiro[(2-hydroxy-3-methyl)indane-1,4′-piperidine] (0.121 g, 0.382 mmol, this was prepared in Preparation 24) in THF (3 ml) was added imidazole (2.6 mg, 0.0382 mmol) and NaH (31 mg, 0.764 mmol, 60% oil dispersion in mineral oil), and the resulting mixture was stirred at 0° C. for 40 minutes. To the mixture was added CS2 at 0° C., and the mixture was stirred for further stirred at 0° C. for 1.5 h. To the mixture was added MeI, and the mixture was stirred at 0° C. for 30 minutes. The reaction was quenched with ice-cooled water, and the product was extracted with EtOAc. The organic layer was dried (Na2SO4)and concentrated. The residue was purified by preparative TLC (1 mm thick plate, Hexane/EtOAc: 5/1) to give 85 mg (55%) of title compound as colorless oil.

¹H NMR (300 MHz, CDCl3) δ 7.30-7.15 (4H, m), 6.10 (1H, d, J=4.2 Hz), 4.10-3.82 (2H, m), 3.42-3.31 (1H, m), 3.25-3.10 (1H, m), 3.03 (1H, ddd, J=2.9 Hz, 11.2 Hz, 13.9 Hz), 2.59 (3H, s), 2.10 (1H, d, J=14.1 Hz), 1.94-1.63 (3H, m), 1.48 (9H, s), 1.43 (3H, d, J=7.3 Hz).

MS (FAB positive) m/z: 408 (M+H)⁺

### Preparation 26

### N-tert-Butoxycarbonylspiro[(3-methyl)indane-1,4′-piperidine]

A solution of N-tert-Butoxycarbonylspiro[{(2-(methylthiocarbonothioyl)oxy)-3-methyl}indan-1,4′-piperidine] (85 mg, 0.210 mmol, this was prepared in Preparation 25), n-Bu3SnH (62 μl, 0.231 mmol), and azobisisobutylronitlile (17 mg, 0.105 mmol) in toluene (3 ml) was heated under reflux for 3 days. After cooling, the reaction mixture was concentrated to give a residue, which was purified by silica gel column chromatography (50 g, Hexane/EtOAc 10/1 as eluent) to give 51 mg (82%) of title compound as colorless oil.

¹H NMR (270 MHz, CDCl3) δ 7.25-7.20 (4H, m), 4.09 (2H, m), 3.23 (1H, ddd, J=7.1 Hz, 7.4 Hz, 16.2 Hz), 3.05-2.83 (2H, m), 2.50 (1H, dd, J=7.6 Hz, 12.7 Hz), 2.04 (1H, dt, J=4.6 Hz, 13.0 Hz), 1.60-1.30 (16H, m, including 9H, s at 1.49 ppm and 3H, d, J=6.8 Hz at 1.33 ppm), 1.33 (3H, d, J=6.8 Hz).

### Preparation 27

### Spiro[(3-methyl)indane-1,4′-piperidine]

To a stirred solution of N-tert-Butoxycarbonylspiro[(3-methyl)indan-1,4′-piperidine] (51 mg, 0.171 mmol, this was prepared in Preparation 26) in CH2Cl2 (2 ml) was added trifluoroacetic acid (1 ml) at 0° C. and the resulting reaction mixture was stirred at room temperature for 2 h. The reaction mixture was evapolated to remove the solvents, poured into a saturated aqueous NaHCO3 solution and extracted with CH2Cl2. The extracts combined were dried (Na2SO4), filtered, and concentrated to give 34 mg (100%) of title compound as colorless oil.

¹H NMR (300 MHz, CDCl3) δ 7.27-7.15 (4H, m), 3.22 (1H, dd, J=7.2 Hz, 14.5 Hz), 3.15-3.00 (2H, m), 2.95-2.77 (2H, m), 2.54 (1H, dd, J=7.7 Hz, 12.8 Hz), 2.34 (1H, br. s), 2.07 (1H, dt, J=4.0 Hz, 12.7 Hz), 1.68-1.15 (7H, m, including 3H, d, J=6.8 Hz at 1.32 ppm).

### Example 72

### 1-[3-[(2S)-2-[(Dimethylamino)carbonyl]-2,3-dihydro-1H-indol-1-yl]-3-oxopropyl]spiro[(3-methyl)indane-1,4′-piperidine]citrate

A mixture of (2S)-1-acryloyl-N,N-dimethyl-2,3-dihydro-1H-indole-2-carboxamide (50 mg, 0.205 mmol, this was prepared in Preparation 22), Spiro[(3-methyl)indan-1,4′-piperidine] (34 mg, 0.171 mmol, this was prepared in Preparation 27), and triethylamine (48 μl, 0.341 mmol) in THF (3 ml) was stirred at reflux temperature for 2 days. The reaction mixture was cooled to room temperature and evapolated to remove the solvent. The residue was purified by silica gel column chromatography (50 g, CH2Cl2/MeOH: 10/1 as eluent) to give 66 mg (87%) of free form of title compound as colorless oil.

¹H NMR (300 MHz, DMSO-d6) δ 8.11 (1H, d, J=7.9 Hz), 7.25-7.13 (6H, m), 6.98 (1H, t, J=7.9 Hz), 5.62 (1H, br. d, J=7.9 Hz), 3.64 (1H, dd, J=11.0 Hz, 16.5 Hz), 3.50-3.23 (2H, m), 3.23-3.08 (4H, m, including 3H, s, at 3.12 ppm), 3.01 (1H, d, J=16.5 Hz), 2.95-2.55 (7H, m, including 3H, s, at 2.88 ppm), 2.55-2.40 (1H, m), 2.30-2.00 (3H, m), 1.60-1.35 (3H, m), 1.35-1.20 (1H, m), 1.26 (3H, d, J=7.0 Hz).

This was converted to citric acid salt according to the procedure described in Example 34 to give 76 mg of title compound as white amorphous solid.

MS (ESI positive) m/z: 446 (M+H)⁺.

IR(KBr): 3400, 2932, 2579, 1734, 1647, 1485, 1404, 1217, 1122, 758 cm⁻¹

Anal. Calcd for C28H35N3O2-C6H8O7-5H2O: C, 59.81; H, 7.09; N, 6.15. Found: C, 59.90; H, 6.76; N, 5.79.

### Example 73

### 1-Methyl-1′-[3-[(2S)-2-[(dimethylamino)carbonyl]-2,3-dihydro-1H-indol-1-yl]-3-oxopropyl]spiro[indoline-3,4′-piperidine]citrate

A mixture of (2S)-1-acryloyl-N,N-dimethyl-2,3-dihydro-1H-indole-2-carboxamide (64 mg, 0.261 mmol, this was prepared in Preparation 22), 1-Methylspiro[indoline-3,4′-piperidine] (39 mg, 0.217 mmol, this was prepared according to known procedure: Simon M. N. Efange et al. J. Med. Chem. 1997, 40, 3905), and triethylamine (45 μl, 0.326 mmol) in THF (3 ml) was stirred at reflux temperature for 1 day. The reaction mixture was cooled to room temperature and evapolated to remove the solvent. The residue was purified by silica gel column chromatography (50 g, Hexane/Acetone: 3/2 then CH2Cl2/MeOH: 10/1 as eluent) to give 51 mg (53%) of free form of title compound as brown oil.

Two isomers with a ratio of 1:1 were observed in CDCl3 solution.

¹HNMR (300 MHz, CDCl3) δ 8.29 (0.5H, d, J=8.1 Hz), 7.31-7.15 (2.5H, m), 7.10 (1H, dt, J=1.1 Hz, 7.5 Hz), 7.05 (1H, m), 7.00 (1H, t, J=8.3 Hz), 6.69 (1H, t, J=7.5 Hz), 6.48 (1H, d, J=7.7 Hz), 5.46 (0.5H, d, J=7.2 Hz), 5.35-5.20 (0.5H, m), 3.69 (0.5H, dd, J=11.0 Hz, 14.9 Hz), 3.46 (0.5H, dd, J=11.2 Hz, 16.3 Hz), 3.25-2.83 (14H, m), 2.76 (3H, s), 2.60-2.43 (1H, m), 2.22 (2H, t, J=11.7 Hz), 2.05-1.88 (2H, m), 1.75 (2H, d, J=13.4 Hz).

This was converted to citric acid salt according to the procedure described in Example 34 to give 136 mg of title compound as brown amorphous solid.

MS (ESI positive) m/z: 447 (M+H)⁺.

IR(KBr): 3398, 2932, 2579, 1732, 1655, 1485, 1406, 1273, 1123, 754 cm⁻¹

Anal. Calcd for C27H34N4O2-C6H8O7-H2O: C, 60.35; H, 6.75; N, 8.53. Found: C, 60.06; H, 6.84; N, 8.63.

### Preparation 28

### 2,3-Dihydro-1′-(3-chloropropyl)spiro[1H-indene-1,4′-piperidine]

To a stirred solution 2,3-Dihydro-1′-(3-hydroxypropyl)spiro[1H-indene-1,4′-piperidine] (0.870 g, 3.55 mmol, this was prepared in Preparation 9) in CHCl3 (30 ml) was added thionyl chloride (0.388 ml, 5.32 mmol) at room temperature and the resulting reaction mixture was refluxed with stirring for 2 h. After cooling, the reaction mixture was poured into a saturated aqueous NaHCO3 solution and extracted with CH2Cl2. The extracts combined were dried (Na2SO4), filtered, and concentrated. The residue was purified by silica gel column chlomatography (200 g, CH2Cl2/MeOH: 20/1 as eluent) to give 0.540 g (58%) of title compound as brown solid.

¹H NMR (270 MHz, CDCl3) δ 7.35-7.28 (1H, m), 7.26-7.17 (3H, m), 3.68 (2H, t, J=6.1 Hz), 3.36 (2H, d, J=11.7 Hz), 3.03-2.90 (4H, m), 2.70 (2H, t, J=12.5 Hz), 2.55-2.30 (4H, m), 2.05 (2H, t, J=7.3 Hz), 1.72 (2H, d, J=14.0 Hz).

### Example 74

### 2,3-Dihydro-11-[3-(3,3-dimethyl-2-oxo-2,3-dihydro-1H-indol-1-yl)propyl]spiro[1H-indene-1,41-piperidine]citrate

A mixture of 2,3-Dihydro-1′-(3-chloropropyl)spiro[1H-indene-1,4′-piperidine] (70 mg, 0.265 mmol, this was prepared in preparation 28), 1,3-Dihydro-3,3,-dimethyl-2H-indol-2-one (51 mg, 0.318 mmol, this was prepared according to known procedure: David W. Robertson et al, J. Med. Chem. 1986, 29, 1832), and KF-A1203 (0.25 g) in CH3CN (8 ml) was stirred at reflux temperature for 1 day. After cooling, the reaction mixture was filtered over celite, and the filtrate was concentrated. The residue was purified by NH-silica gel column chlomatography(50 g, Hexane/EtOAc: 9/1) to give 89 mg (87%) of free form of title compound as colorless oil.

¹H NMR (270 MHz, CDCl3) δ 7.30-7.10 (6H, m), 7.04 (1H, dt, J=1.0 Hz, 7.4 Hz), 6.95 (1H, d, J=7.8 Hz), 3.79 (2H, t, J=7.1 Hz), 2.92-2.82 4H, m, including 2H, t, J=7.3 Hz at 2.88 ppm), 2.44 (2H, t, J=6.9 Hz), 2.14 (2H, br. t, J=10.1 Hz), 2.02-1.83 (6H, m, including 2H, t, J=7.4 Hz at 1.99 ppm), 1.54 (2H, d, J=12.9 Hz), 1.37 (6H, s).

This was converted to citric acid salt according to the procedure described in Example 34 to give 88 mg of title compound as white amorphous solid.

MS (ESI positive) m/z: 389 (M+H)⁺.

IR(KBr): 3400, 2934, 1709, 1613, 1387, 1366, 1200, 762 cm⁻¹

Anal. Calcd for C26H32N2O-C6H8O7-2H2O: C, 62.32; H, 7.19; N, 4.54. Found: C, 62.27; H, 6.73; N, 4.34.

### Example 75

### 2,3-Dihydro-1′-13-(3,3-dimethyl-2,3,-dihydro-1H-indol-1-yl)-3-oxopropyl]spiro[1H-indene-1,4′-piperidine] citrate

To a stirred solution of 3,3-dimethyl-2,3-dihydro-1H-indole (100 mg, 0.679 mmol, this was prepared according to known procedure: Andrew Kucerovy et al, Synth. Commun. 1992, 22, 729) and triethylamine (0.28 ml, 2.04 mmol) in CH2Cl2 (5 ml) was added 2,3-dihydro-1′-[2-(chloroformyl)ethyl]spiro[1H-indene-1,4′-piperidine]hydrochloride (0.235 g, 0.747 mmol, this was prepared in Preparation 3) at 0° C. and the resulting reaction mixture was stirred at room temperature for 1 day. The reaction mixture was poured into a saturated aqueous NaHCO3 solution and extracted with EtOAc. The organic layer was washed with saturated aqueous NaHCO3 solution and brine, dried (Na2SO4), filtered, and concentrated. The residue was purified by NH-silica gel column chromatography (50 g, Hexane/EtOAc: 5/1-3/1 as eluent) to give 0.227 g (86%) of free form of title compound as oil.

¹H NMR (270 MHz, CDCl3) δ 8.21 (1H, d, J=8.1 Hz), 7.25-7.12 (6H, m), 7.06 (1H, t, J=7.4 Hz), 3.82 (2H, s), 3.00-2.85 (6H, m), 2.70 (2H, t, J=7.7 Hz), 2.29 (2H, dt, J=2.5 Hz, 12.4 Hz), 2.08-1.88 (4H, m, including 2H, t, J=7.4 Hz at 2.03 ppm), 1.59 (2H, d, J=16.2 Hz), 1.36 (6H, s).

This was converted to citric acid salt according to the procedure described in Example 34 to give 0.267 g of title compound as white amorphous solid.

MS (ESI positive) m/z: 389 (M+H)⁺.

IR(KBr): 2955, 1724, 1665, 1597, 1483, 1421, 1286, 752 cm⁻¹

Anal. Calcd for C26H32N2O—C6H8O7-0.3H2O: C, 65.58; H, 6.98; N, 4.78. Found: C, 65.62; H, 7.00; N, 4.85.

### Example 76

### 2,3-Dihydro-1′-[3-(2,3,-dihydro-4H-1,4-benzothiazin-4-yl)-3-oxopropyl]spiro[1H-indene-1,4′-piperidine]citrate

To a stirred solution of 2,3-Dihydro-2H-1,4-benzothiazine (46 mg, 0.302 mmol, this was prepared according to known procedure: Saverio Florio et al, J. Heterocycl. Chem. 1982, 19, 237) and triethylamine (0.13 ml, 0.907 mmol) in CH2Cl2 (3 ml) was added 2,3-dihydro-1′-[2-(chloroformyl)ethyl]spiro[1H-indene-1,4′-piperidine]hydrochloride (95 mg, 0.302 mmol, this was prepared in Preparation 3) at 0° C. and the resulting reaction mixture was stirred at room temperature for 1 day. The reaction mixture was poured into a saturated aqueous NaHCO3 solution and extracted with EtOAc. The organic layer was washed with saturated aqueous NaHCO3 solution and brine, dried (Na2SO4), filtered, and concentrated. The residue was purified by preparative TLC (1 mm thick plate, CH2Cl2/MeOH/: 10/1) to give 2.9 mg (2.4%) of free form of title compound as oil.

¹H NMR (270 MHz, CDCl3) δ 7.30-7.07 (8H, m), 4.00 (2H, m), 3.25 (2H, t, J=5.77 Hz), 2.87 (2H, t, J=7.3 Hz), 2.74 (6H, m), 2.17 (2H, br. t, J=9.6 Hz), 2.05-1.70 (6H, m, including 2H, t, J=7.4 Hz at 1.96 ppm), 1.49 (2H, d, J=13.0 Hz).

MS (EI direct) m/z: 392 (M)⁺.

This was converted to citric acid salt according to the procedure described in Example 34 to give 5.9 mg of title compound as red amorphous solid.

MS (ESI positive) m/z: 393 (M+H)⁺.

### Example 77

### 2,3-Dihydro-1′-[3-[3-(hydroxymethyl)-2,3,-dihydro-4H-1,4-benzoxazin-4-yl]-3 oxopropyl]spiro[1H-indene-1,4′-piperidine]citrate

To a stirred solution of 3,4-Dihydro-2H-1,4-benzoxiazin-3-ylmethanol (20 mg, 0.124 mmol, this was prepared according to known procedure:G. W. H. Potter et al, J. Heterocycl. Chem. 1972, 9, 299) and triethylamine (52 μl, 0.371 mmol) in CH2Cl2 (2 ml) was added 2,3-dihydro-1′-[2-(chloroformyl)ethyl]spiro[1H-indene-1,4′-piperidine]hydrochloride (39 mg, 0.124 mmol, this was prepared in Preparation 3) at 0° C. and the resulting reaction mixture was stirred at room temperature for 20 h. The reaction mixture was poured into a saturated aqueous NaHCO3 solution and extracted with EtOAc. The organic layer was washed with saturated aqueous NaHCO3 solution and brine, dried (Na2SO4), filtered, and concentrated. The residue was purified by preparative TLC (0.5 mm thick plate, CH2Cl2/MeOH/: 10/1) to give 7.0 mg (14%) of free form of title compound as oil.

¹H NMR (270 MHz, CDCl3) δ 7.24-7.13 (4H, m), 6.78 (2H, t, J=8.4 Hz), 6.70-6.57 (2H, m), 4.28-4.15 (3H, m), 4.10 (2H, dd, J=5.3 Hz, 10.7 Hz), 3.80-3.65 (1H, m), 2.97-2.84 (4H, m, including 2H, t, J=7.3 Hz at 2.89 ppm), 2.84-2.73 (2H, m), 2.61 (2H, t, J=6.6 Hz), 2.30-2.16 (2H, m), 2.05-1.85(4H, m, including 2H, t, J=7.4 Hz at 2.00 ppm), 1.55 (2H, d, J=13.2 Hz).

This was converted to citric acid salt according to the procedure described in Example 34 to give 9.8 mg of title compound as white amorphous solid.

MS (ESI positive) m/z: 407 (M+H)⁺.

### Preparation 29

### 2,3-Dihydro-1′-[2-(tert-butoxycarbonyl)amino-3-ethoxy-3-oxopropyl]spiro[1H-indene-1,4′-piperidine]

A mixture of 2,3-Dihydrospiro[1H-indene-1,4′-piperidine]hydrochloride (0.352 g, 1.57 mmol, this was prepared according to known procedure: M. S. Chambers et al, J. Med. Chem. 1992, 35, 2033), Methyl 2-[(tert-butoxycarbonyl)amino]acrylate (0.288 g, 1.43 mmol, this was prepared according to known procedure: Paula M. T. Ferreira et al, J. Chem. Soc. Perkin Trans. 1, 1999, 24, 3697), and triethylamine (0.30 ml, 2.15 mmol) in EtOH (15 ml) was stirred at reflux temperature for 1 day. The reaction mixture was cooled to room temperature and evapolated to remove the solvent. The residue was purified silica gel column chromatography (50 g, Hexane/EtOAc: 9/1-4/1 as eluent) to give 0.172 g (31%) of title compound as yellow oil.

¹H NMR (300 MHz, CDCl3) δ 7.23-7.10 (4H, m), 5.40 (1H, m), 4.38-4.10 (3H, m), 2.90-2.65 (6H, m, including 2H, t, J=7.3 Hz at 2.88 ppm), 2.36-2.22 (2H, m), 1.98 (2H, t, J=7.4 Hz), 1.89 (2H, dt, J=3.3 Hz, 12.5 Hz), 1.55-1.45 (11H, m, including 9H, s, at 1.47 ppm), 1.30 (3H, t, J=7.2 Hz).

### Preparation 30

### 2,3-Dihydro-1′-[3-(indolin-1-yl)-2-(tert-butoxycarbonyl)amino-3-oxopropyl]spiro[1H-indene-1,4′-piperidine]

A mixture of 2,3-Dihydro-1′-[2-(tert-butoxycarbonyl)amino-3-ethoxy-3-oxopropyl]spiro[1H-indene-1,4′-piperidine] (0.345 g, 0.889 mmol, this was prepared in Preparation 29), and 2N NaOH (0.67 ml, 1.333 mmol) in THF-MeOH (6 ml-2 ml) was stirred at 60° C. for 2 h. The reaction mixture was cooled to room temperature, neutralized by 2N HCl, and evapolated to give crude corresponding carboxylic acid. This was used for the next step without purification.

A mixture of this carboxylic acid, indoline (0.1999 ml, 0.178 mmol), WSC (0.341 g, 0.178 mmol), HOBt (0.242 g, 0.178 mmol), and triethylamine (0.372 ml, 0.267 mmol) in CH2Cl2 (10 ml) was stirred at room temperature for 3 days. The reaction mixture was diluted with saturated aqueous NaHCO3 solution and extracted with CH2Cl2. The extracts combined were dried (Na2SO4), filtered, and concentrated. The residue was purified by silica gel column chromatography (50 g, Hexane/Acetone: 5/1) to give 0.288 g (68%, 2steps) of title compound as colorless oil.

¹H NMR (270 MHz, CDCl3) δ 8.24 (1H, d, J=8.1 Hz), 7.26-7.10 (6H, m), 7.05 (1H, t, J=7.4 Hz), 5.42 (1H, br.t, J=7.8 Hz), 4.73 (1H, dt, J=6.9 Hz, 7.6 Hz), 4.36 (2H, dt, J=2.5 Hz, 6.8 Hz), 3.25 (2H, t, J=8.4 Hz), 2.87 (2H, t, J=7.5 Hz), 3.07-2.65 (4H, m, including 2H, t, J=6.8 Hz at 2.73 ppm), 2.45-2.20 (2H, m), 2.00-1.75 (4H, m, including 2H, t, J=7.1 Hz at 1.98 ppm), 1.60-1.35 (11H, m, including 9H, s, at 1.45 ppm).

### Example 78

### 2,3-Dihydro-1′-[2-amino-3-(indolin-1-yl)-3-oxopropyl]spiro[1H-indene-1,4′-piperidine]citrate

To a stirred solution of 2,3-Dihydro-1′-[3-(indolin-1-yl)-2-(tert-butoxycarbonyl)amino-3-oxopropyl]spiro[1H-indane-1,4′-piperidine] (0.288 g, 0.605 mmol, this was prepared in Preparation 30) in CH2Cl2 (4 ml) was added trifluoroacetic acid (2 ml) at 0° C. and the resulting reaction mixture was stirred at room temperature for 1 h. The reaction mixture was evapolated to remove the solvents, poured into a saturated aqueous NaHCO3 solution and extracted with CH2Cl2. The extracts combined were dried (Na2SO4), filtered, and concentrated. The residue was purified by silica gel column chromatography (50 g, CH2Cl2/MeOH: 10/1) to give 0.225 g (99%) of title compound as colorless oil.

¹H NMR (300 MHz, CDCl3) δ 8.26 (1H, d, J=8.3 Hz), 7.25-7.13 (6H, m), 7.04 (1H, dt, J=0.9 Hz, 7.3 Hz), 4.22 (2H, t, J=8.3 Hz), 3.88 (1H, dd, J=4.6 Hz, 8.3 Hz), 3.23 (2H, t, J=8.4 Hz), 3.00-2.85 (2H, m), 2.89 (2H, t, J=7.5 Hz), 2.65 (1H, dd, J=4.8 Hz, 12.7 Hz), 2.54 (1H, dd, J=8.8 Hz, 12.8 Hz), 2.42 (1H, br. t, J=9.9 Hz), 2.24 (1H, bt. t, J=11.4 Hz), 2.11 (2H, br. s), 2.00 (2H, t, J=7.3 Hz), 2.00-1.83 (2H, m), 1.60-1.47 (2H, m).

This compound (46 mg) was converted to citric acid salt according to the procedure described in Example 34 to give 56 mg of title compound as white amorphous solid. MS (ESI positive) m/z: 376 (M+H)⁺.

IR(KBr): 3400, 2935, 1719, 1665, 1560, 1485, 1437, 1211, 758 cm⁻¹

Anal. Calcd for C24H29N3O—C6H8O7-1.8H2O: C, 60.05; H, 6.82; N, 7.00. Found: C, 60.17; H, 6.71; N, 6.66.

### Example 79

### 2,3-Dihydro-1′-[3-(indolin-1-yl)-2-dimethylamino-3-oxopropyl]spiro[1H-indene-1,4′-piperidine]citrate

A mixture of 2,3-Dihydro-1′-[2-amino-3-(indolin-1-yl)-3-oxopropyl]spiro[H-indene-1,4′-piperidine] (52 mg, 0.140 mmol, this was prepared in Example 78), 37% formaldehyde solution in water (51 μl, 0.698 mmol) and CH3CN (2 ml) was added NaBH3CN (26 mg, 0.419 mmol) at 0° C., and the resulting mixture was stirred at room temperature for 1 day. The reaction mixture was quenched with water, diluted with saturated aqueous NaHCO3 solution and extracted with CH2Cl2. The extracts combined were dried (Na2SO4), filtered, and concentrated. The residue was purified by preparative TLC (1 mm thick plate, CH2Cl2/MeOH/: 10/1) to give 34 mg (61%) of free form of title compound as colorless oil.

¹H NMR (270 MHz, CDCl3) δ 8.30(1H, d, J=8.6 Hz), 7.26-7.10 (6H, m), 7.02 (1H, t, J=7.4 Hz), 4.39 (1H, dd, J=9.9 Hz, 19.0 Hz), 4.19 (1H, dd, J=10.1 Hz, 18.8 Hz), 3.60 (1H, dd, J=4.3 Hz, 7.9 Hz), 3.21 (2H, t, J=8.4 Hz), 3.09 (1H, dd, J=4.1 Hz, 12.7 Hz), 2.97 (1H, br. d, J=11.7 Hz), 2.87 (2H, t, J=7.3 Hz), 2.92-2.78 (1H, m), 2.71 (1H, dd, J=3.8 Hz, 12.7 Hz), 2.42 (6H, s), 2.33 (2H, br.t, J=12.0 Hz), 1.99 (2H, t, J=7.4 Hz), 2.00-1.80 (2H, m), 1.50 (2H, br. t, J=13.4 Hz).

This was converted to citric acid salt according to the procedure described in Example 34 to give 20 mg of title compound as white amorphous solid.

MS (ESI positive) m/z: 404 (M+H)⁺.

IR(KBr): 3400, 2941, 2572, 1719, 1655, 1597, 1483, 1420, 1188, 758 cm⁻¹

Anal. Calcd for C26H33N3O—C6H8O7-2H2O: C, 60.84; H, 7.18; N, 6.65. Found: C, 61.15; H, 6.94; N, 6.50.

### Preparation 31

### Benzyl 1-acryloyl-1,2,3,4-tetrahydro-2-quinolinecarboxylate

To a stirred solution of benzyl 1,2,3,4-tetrahydro-2-quinolinecarboxylate [100.0 mg, 0.374 mmol, this was prepared according to known procedure: R Nagata, et al, J. Med. Chem. 1994, 37, 3956] in CH2Cl2 (5 ml) was added triethylamine (0.094 ml, 0.673 mmol) and the resulting mixture was cooled at −30° C. To the reaction mixture was added chloropropionyl chloride (57.0 mg, 0.449 mmol) and was stirred at −30° C.˜−20° C. for 45 min. Then to the reaction mixture was added triethylamine (0.052 ml, 0.374 mmol) and chloropropionyl chloride (47.5 mg, 0.374 mmol) and stirred for min at −30° C. The reaction mixture was poured into a saturated aqueous NaHCO3 solution and extracted with CH2Cl2 (15 ml×3). The extracts combined were washed with brine, dried (Na2SO4), filtered, and concentrated. The residue was purified by preparative TLC (1 mm thick silica gel plate: n-Hexane/AcOEt:3/1) to give 93.1 mg (78%) of the title product as pale yellow oil.

MS (EI direct) m/z:321(M)⁺

### Preparation 32

### 2,3-Dihydro-1′-{3-[2-[(benzyloxy)carbonyl]-3,4-dihydro-[(2B)-quinolinyl]-3-oxopropyl}spiro[1H-indene-1,4′-piperidine]

A mixture of 2,3-dihydrospiro[1H-indene-1,4′-piperidine]hydrochloride (64.9 mg, 0.290 mmol, this was prepared according to known procedure:M. S. Chambers et al, J. Med. Chem. 1992, 35, 2033), benzyl 1-acryloyl-1,2,3,4-tetrahydro-2-quinolinecarboxylate (93.1 mg, 0.290 mmol), and triethylamine (0.061 ml, 0.435 mmol) was stirred at 60° C. for 15 h. Then to the reaction mixture was added triethylamine (0.061 ml, 0.435 mmol) and stirred at 90° C. for Id. The reaction mixture was poured into a saturated aqueous NaHCO3 solution and extracted with AcOEt (20 ml×3). The extracts combined were dried (Na2SO4), filtered, and concentrated. The resulting residue was purified by preparative TLC (1 mm thick silica gel plate: CH2Cl2/MeOH:25/1) to afford 57.5 mg (39%) of title product as pale yellow oil.

¹H NMR (270 MHz, CDCl3) δ7.34-7.13 (13H, m), 5.31-5.25 (1H, m), 5.11 (2H, s), 0.89-2.50 (11H, m), 2.16-2.12 (2H, m), 1.98-1.73 (5H, m), 1.50-1.46 (2H, m) (EI direct) m/z: 508(M)⁺

### Preparation 33

### 2,3-Dihydro-1′-13-{2-carboxy-3,4-dihydro-[(2R)-quinolinyl}-3-oxopropyl]spiro[1H-indene-1,4′-piperidine]

To a stirred solution of 2,3-dihydro-1′-{3-[2-[(benzyloxy)carbonyl]-3,4-dihydro-1(2H)-quinolinyl]-3-oxopropyl}spiro[1H-indene-1,4′-piperidine] (57.5 mg, 0.113 mmol) in THF (0.5 ml) and MeOH (0.5 ml) was added 2N NaOH (0.23 ml, 0.460 mmol) at room temperature. After 2 h stirring at room temperature, the reaction mixture was dissolved to AcOEt, washed with 1N—HCl (4 ml). The extracts combined were dried (Na2SO4), filtered, and concentrated to give 49.0 mg (100%) of crude compound as a white solid.

MS (ESI positive) mn/z: 419 (M+H)⁺

MS (ESI negative) m/z: 417 (M−H)⁺

### Example 80

### 2,3-Dihydro-1′-{3-[2-(aminocarbonyl)-3,4-dihydro-1 (2H)-quinolinyl]-3-oxopropyl}spiro[1H-indene-1,4′-piperidine]citrate

To a stirred suspension of 2,3-dihydro-1′-[3-{2-carboxy-3,4-dihydro-[(2H)-quinolinyl}-3-oxopropyl]spiro[1H-indene-1,4′-piperidine] (49.0 mg, 0.117 mmol) in MeCN (6 ml) was added 1,1′-carbonyldiimidazole (22.7 mg, 0.140 mmol) and triethylamine (0.020 ml, 0.140 mmol) at room temperature and resulting mixture was stirred at 70° C. for 2 h. To a reaction mixture was added 25% NH4OH (1.5 ml) and stirred at 70° C. for 2 h. Then the reaction mixture was diluted with saturated aqueous NaHCO3 solution, and extracted with CH2Cl2 (20 ml×3). The extracts combined were dried (Na2SO4), filtered, and concentrated. The residue was purified by preparative TLC (1 mm thick plate, CH2Cl2/MeOH:10/1, 2 times developed) to afford 17.4 mg (36%) of free base as colorless oil.

¹H NMR (270 MHz, CDCl3) δ7.21-7.15 (8H, m), 6.68 (2H, br), 5.25-5.19 (1H, m), 2.90-1.76 (18H, m), 1.51-1.46 (2H, m)

MS (ESI positive) m/z: 418 (M+H)⁺

This was dissolved in mixed solvent of CH2Cl2 (1 ml) and MeOH (1 ml) followed by addition of citric acid (7.3 mg, 0.038 mmol) and resulting mixture was stirred for 2h. After concentration, the residue was solidified by adding CH2Cl2-hexane. The resulting solid was collected by filtration and washed with ether to give 18.2 mg of citrate as an yellow amorphous solid.

IR(KBr): 2937, 2575, 1653, 1396, 1204, 760 cm⁻¹

Anal. Calcd for C26H31N3O2-C6H8O7-1.5H2O: C, 60.37; H, 6.65; N, 6.60. Found: C, 60.36; H, 6.41; N, 6.46

### Example 81

### 2,3-Dihydro-1′-(3-{(2S)-2-[(4-hydroxy-1-piperidinyl)carbonyl]-2,3-dihydro-1H-indol-1-yl}-3-oxopropyl)spiro[1H-indene-1,4′-piperidine]citrate

A mixture of 2,3-dihydro-1′-[3-(2-(S)-carboxyindolin-1-yl)-3-oxopropyl]spiro[1H-indene-1,4′-piperidine] (70.0 mg, 0.173 mmol, this was prepared in Preparation 9), 4-hydroxypiperidine (52.5 mg, 0.519 mmol), WSC (66.3 mg, 0.346 mmol), HOBt (46.8 mg, 0.346 mmol), and triethylamine (72 μl, 0.519 mmol) in CH2Cl2 (5 ml)—DMF (5 ml)—THF (1 ml) was stirred at room temperature for 21 h. The reaction mixture was diluted with saturated aqueous NaHCO3 solution and extracted with CH2Cl2. The extracts combined were dried (Na2SO4), filtered, and concentrated. The residue was purified by preparative TLC (1 mm thick plate, AcOEt/PrOH25% NH4OH:200/40/15) to give 63.5 mg (75%) of free base as a white solid. This compound showed broadened spectra in proton NMR.

This was converted to citric acid salt similar to that described in Example 34 to give 76.1 mg of citrate as a white solid.

MS (ESI positive) m/z: 488 (M+H)⁺

IR(KBr): 3393, 2943, 1728, 1653, 1213, 758 cm⁻¹

Anal. Calcd for C30H37N3O3-C6H8O7-0.2H₂O-0.5CH2Cl2: C, 60.40; H, 6.44; N, 5.79. Found: C, 60.18; H, 6.06; N, 5.81

### Example 82

### 2,3-Dihydro-1′-[3-((2S)-2-{1[4-(aminocarbonyl)-1-piperidinyl]carbonyl}-2,3-dihydro-1H-indol-1-yl)-3-oxopropyl]spiro[1H-indene-1,4′-piperidine]citrate

This was prepared according to the procedure described in Example 81 using isonipecotamide instead of 4-hydroxypiperidine. 58.5 mg (66%) of free base was obtained as yellow oil. This compound showed broadened spectra in proton NMR. This was converted to citric acid salt similar to that described in Example 34 to give 66.5 mg of citrate as a white solid.

MS (ESI positive) m/z: 515 (M+H)⁺

IR(KBr): 3366, 2932, 1719, 1601, 1211, 760 cm⁻¹

Anal. Calcd for C31H38N4O3-C6H8O7-2H2O: C, 59.83; H, 6.78; N, 7.54. Found: C, 59.73; H, 6.53; N, 7.53

### Example 83

### 2,3-Dihydro-1′-{3-oxo-3-[(2S)-2-(1-piperazinylcarbonyl)-2,3-dihydro-1H-indol-1-yl propyl}spiro[1H-indene-1,4′-piperidine]citrate

This was prepared according to the procedure described in Example 81 using Boc piperazine instead of 4-hydroxypiperidine followed by removal of Boc group by treatment of TFA and basic workup. 32.1 mg (30%) of free base was obtained as pale yellow oil. This compound showed broadened spectra in proton NMR except for the following peaks.

¹H NMR (270 MHz, CDCl3) δ 8.32-8.30 (0.3H, m), 7.03-6.98 (1H, m), 5.50-5.47 (0.5H, m), 2.52 (1H, m), 2.26 (2H, m), 1.59-1.54 (2H, m)

This was converted to citric acid salt similar to that described in Example 34 to give 39.7 mg of citrate as a white solid.

MS (ESI positive) rn/z: 473 (M+H)⁺

IR(KBr): 3422, 2941, 1653, 1034, 758 cm⁻¹

Anal. Calcd for C29H36N4O2-C6H8O7-1.7H2O: C, 60.45; H, 6.87; N, 8.06. Found: C, 60.44; H, 6.64; N, 7.89

### Example 84

### 2,3-Dihydro-1′-(3-oxo-3-{(2S)-2-[(4-pyridinylamino)carbonyl]-2,3-dihydro-1H-indol-1-yl}propyl)spiro [1H-indene-1,4′-piperidine]citrate

This was prepared according to the procedure described in Example 81 using 4-aminopyridine instead of 4-hydroxypiperidine. 50.7 mg (61%) of free base was obtained as yellow oil. This compound showed broadened spectra in proton NMR except for the following peaks.

¹H NMR (270 MHz, CDCl3) δ 9.77 (0.2H, br), 8.48-8.45 (2H, m), 7.47-7.45 (2H, m), 2.32-2.23 (2H, m), 2.02-1.89 (5H, m), 1.58-1.54 (2H, m).

This was converted to citric acid salt similar to that described in Example 34 to give 55.3 mg of citrate as a white solid.

MS (ESI positive) m/z: 481(M+H)⁺

MS (ESI negative) m/z: 479(M−H)⁺

IR(KBr): 3393, 2932, 1717, 1597, 1184, 835, 758 cm⁻¹

Anal. Calcd for C30H32N4O2-C6H8O7-2H2O: C, 61.01; H, 6.26; N, 7.90. Found: C, 61.19; H, 6.04; N, 7.68.

### Example 85

### 2,3-Dihydro-1′-(3-oxo-3-{(2S)-2-[(1,3-thiazol-2-ylamino)carbonyl]-2,3-dihydro-1H-indol-1-yl}propyl)spiro[1H-indene-1,4′-piperidine]citrate

This was prepared according to the procedure described in Example 81 using 2-aminothiazole instead of 4-hydroxypiperidine. 61.2 mg (73%) of free base was obtained as yellow oil. This compound showed broadened spectra in proton NMR except for the following peaks.

¹H NMR (270 MHz, CDCl3) δ 7.45-7.43 (1H, m), 7.25-7.07(8H, m), 6.97-6.96 (1H, m), 2.31-2.23 (2H, m), 2.03-1.90 (5H, m), 1.56-1.51 (2H, m)

This was converted to citric acid salt similar to that described in Example 34 to give 66.1 mg of citrate as a white solid.

MS (ESI positive) m/z: 487(M+H)⁺

MS (ESI negative) m/z: 485(M−H)⁺

IR(KBr): 2941, 1541, 758 cm⁻¹

Anal. Calcd for C28H30N4O2S—C₆H8O7-1.5H2O: C, 57.86; H, 5.86; N, 7.94. Found: C, 57.66; H, 5.80; N, 7.71

### Example 86

### 2,3-Dihydro-1′-(3-{(2S)-2-[(4-amino-1-piperidinyl)carbonyl]-2,3-dihydro-1H-indol-1-yl}-3-oxopropyl)spiro[1H-indene-1,4′-piperidine]citrate

This was prepared according to the procedure described in Example 81 using 4-tert-butoxycarbonylaminopiperidine (This was prepared according to known procedure: Carling, Robert W. et al, J. Med. Chem., 1999, 42, 2706) instead of 4-hydroxypiperidine followed by removal of Boc group by treatment of TFA and basic workup. 81.8 mg (66%) of free base was obtained as pale yellow oil.

This compound showed broadened spectra in proton NMR.

This was converted to citric acid salt similar to that described in Example 34 to give 96.2 mg of citrate as a white solid.

MS (ESI positive) m/z: 487 (M+H)⁺

IR(KBr): 2937, 1638, 1219, 758 cm⁻¹

Anal. Calcd for C30H38N4O2-C6H8O7-2H2O: C, 60.49; H, 7.05; N, 7.84. Found: C, 60.41; H, 6.95; N, 7.79

### Example 87

### 2,3-Dihydro-1′-[3-((2S)-2-{[4-(dimethylamino)-1-piperidinyl]carbonyl}-2,3-dihydro-1H-indol-1-yl)-3-oxopropyl]spiro[1H-indene-1,4′-piperdine]citrate

To a stirred solution of 2,3-dihydro-1′-(3-{2-(S)-2-[(4-amino-1-piperidinyl)carbonyl]-2,3-dihydro-1H-indol-1-yl}-3-oxopropyl)spiro[1H-indene-1,4′-piperidine] (66.0 mg, 0.136 mmol, this was prepared in Example 86.) and 37% formic acid (51 μl, 0.680 mmol) in MeCN (4 ml) was added sodium cyanoborohydride (13.7 mg, 0.218 mmol) at 0° C. and resulting mixture was stirred at room temperature for 18 h. Then, to a reaction mixture was added sodium cyanoborohydride (13.7 mg, 0.218 mmol) and stirred at room temperature for 22 h. Then the reaction mixture was diluted with saturated aqueous NaHCO3 solution, and extracted with CH2Cl2 (20 ml×3). The extracts combined were dried (Na2SO4), filtered, and concentrated. The residue was purified by preparative TLC (1 mm thick plate, AcOEt/PrOH/25% NH4OH:10/2/1, 2 times developed) to afford 36.9 mg (53%) of free base as pale yellow oil. This compound showed broadened spectra in proton NMR.

This was converted to citric acid salt similar to that described in Example 34 to give 44.8 mg of citrate as a white solid.

MS (ESI positive) m/z: 515 (M+H)⁺

IR(KBr):3422, 2937, 1653, 762 cm⁻¹

Anal. Calcd for C32H42N4O2-C6H8O7-1.7H2O: C, 61.89; H, 7.30; N, 7.60. Found: C, 61.94; H, 7.19; N, 7.84

### Example 88

### 2,3-Dihydro-1′-(3-oxo-3-{(2S)-2-[(2-pyridinylamino)carbonyl]-2,3-dihydro-1H-indol-1-yl}propyl)spiro[1H-indene-1,4′-piperidine]citrate

This was prepared according to the procedure described in Example 81 using 2-aminopyridine instead of 4-hydroxypiperidine. 14.6 mg (17%) of free base was obtained as yellow oil. This compound showed broadened spectra in proton NMR except for the following peaks.

¹H NMR (270 MHz, CDCl3) δ 8.26-8.06 (3H, m), 7.66 (1H, m), 7.45-7.39 (1H,m), 6.67-6.62 (1H, m), 6.51-6.48 (1H, m), 2.26 (2H, m), 1.55 (2H, m)

This was converted to citric acid salt similar to that described in Example 34 to give 15.5 mg of citrate as a white solid.

MS (ESI positive) m/z: 481 (+H)⁺

MS (ESI negative) m/z: 479(M−H)⁺

IR(KBr):2936, 1701, 1437, 758 cm⁻¹

Anal. Calcd for C30H32N4O2-C6H8O7-1H2O: C, 62.60; H, 6.13; N, 8.11. Found: C, 62.75; H, 6.24; N, 7.78

### Example 89

### 2,3-Dihydro-1′-(3-{(2S)-2-[(diethylamino)carbonyl]-2,3-dihydro-1H-indol-1-yl}-3-oxopropyl)spiro[1H-indene-1,4′-piperidine]citrate

This was prepared according to the procedure described in Example 81 using diethylamine instead of 4-hydroxypiperidine. 91.5 mg (67%) of free base was obtained as yellow oil. This compound showed broadened spectra in proton NMR except for the following peaks.

¹H NMR (270 MHz, CDCl3) δ 8.31-8.28 (0.3H, m), 7.02-6.96 (1H, m), 2.04-1.94 (4H,m), 1.59-1.54 (2H, m).

This was converted to citric acid salt similar to that described in Example 34 to give 118.1 mg of citrate as a white solid.

MS (ESI positive) m/z: 460(M+H)⁺

IR(KBr): 1728, 1645, 757 cm⁻¹

Anal. Calcd for C29H37N3O2-C6H8O7-1.5H2O: C, 61.93; H, 7.13; N, 6.19. Found: C, 62.23; H, 7.39; N, 5.87

### Example 90

### 2,3-Dihydro-1′-[3-((2S)-2-{[ethyl(methyl)amino]carbonyl}-2,3-dihydro-1H-indol-1-yl)-3-oxopropyl]spiro[1H-indene-1,4′-piperidine]citrate

This was prepared according to the procedure described in Example 81 using N—ethylmethylamine instead of 4-hydroxypiperidine. 40.3 mg (30%) of free base was obtained as colorless oil. This compound showed broadened spectra in proton NMR except for the following peaks.

¹H NMR (270 MH, CDCl3) δ 8.31-8.28 (0.3H, m), 7.02-6.97 (1H, m), 2.05-1.99 (4H,m), 1.60-1.56 (2H, m).

This was converted to citric acid salt similar to that described in Example 34 to give 45.6 mg of citrate as a white solid.

MS (ESI positive) m/z: 446(M+H)⁺

IR(KBr):3435, 2937, 1728, 1653, 1485, 1414, 758 cm⁻¹

Anal. Calcd for C28H35N3O2-C6H8O7-1H2O: C, 62.28; H, 6.92; N, 6.41. Found: C, 62.05; H, 7.02; N, 6.04

### Preparation 34

### 2,3-Dihydro-1′-[3-ethoxy-1-methyl-3-oxopropyl]spiro[1H-indene-1,4′-piperidine]

To a stirred solution of 2,3-dihydrospiro[1H-indene-1,4′-piperidine](243.5 mg, 1.300 mmol, this was prepared according to known procedure:M. S. Chambers et al, J. Med. Chem. 1992, 35, 2033) and ethylacetoacetate (338.4 mg, 2.600 mmol) in CH2Cl2 (20 ml) was added sodium triacetoxyborohydride (826.6 mg, 3.900 mmol) and acetic acid (0.22 ml, 3.90 mmol) at 0° C. Then the reaction mixture was stirred at room temperature for 8h. Then to the reaction mixture was added ethylacetoacetate (169.2 mg, 1.300 mmol), sodium triacetoxyborohydride (413.3 mg, 1.950 mmol) and acetic acid (0.11 ml, 1.950 mmol) in CH2Cl2 (10 ml) and stirred for 14 h at room temperature. Then to the reaction mixture was added ethylacetoacetate (169.2 mg, 1.300 mmol), sodium triacetoxyborohydride (413.3 mg, 1.950 mmol) and acetic acid (0.11 ml, 1.950 mmol) and stirred at room temperature for 9 h. Then to the reaction mixture was added ethylacetoacetate (169.2 mg, 1.300 mmol), sodium triacetoxyborohydride (413.3 mg, 1.950 mmol) and acetic acid (0.11 ml, 1.950 mmol) and stirred at room temperature for 23 h. The reaction mixture was poured into a saturated aqueous NaHCO3 solution and extracted with CH2Cl2 (50 ml×3). The extracts combined were washed with H2O, dried (Na2SO4), filtered, and concentrated. The residue was purified by silica gel column chromatography (n-Hexane/AcOEt:3/1 as eluent) to afford 194.9 mg (50%) of title compound as colorless oil. However, this product was contained ethyl acetoacetate. It could not be assigned in proton NMR except for the following peaks.

¹H NMR (270 MHz, CDCl3) δ 7.21-7.13 (4H, m), 3.26-3.16 (1H, m), 2.91-2.61 (7H, m), 2.27 (1H, dd, J=14.2, 8.4 Hz), 2.06-1.83 (5H, m), 1.57-1.52 (2H, m), 1.12 (3H, d, J=6.6 Hz).

### Preparation 35

### 2,3-Dihydro-1′-[2-carbonyl-1-methylethyl]spiro[1H-indene-1,4′-piperidine]hydrochloride

This was prepared according to the procedure described in Preparation 2 using 2,3-dihydro-1′-[3-ethoxy-1-methyl-3-oxopropyl]spiro[1H-indene-1,4′-piperidine] (194.9 mg, 0.647 mmol) instead of 2,3-dihydro-1′-[2-(ethoxycarbonyl)ethyl]spiro[1H-indene-1,4′-piperidine]. 49.3 mg (25%) of title compound was obtained as a white solid.

MS (ESI positive) m/z: 274(M+H)⁺

MS (ESI negative) m/z: 272(M−H)⁺

### Example 91

### 2,3-Dihydro-1′-[3-(2,3-dihydro-1H-indol-1-yl)-1-methyl-3-oxopropyl]spiro[1H-indene-1,4′-piperidine]citrate

This was prepared according to the procedure described in Preparation 3 using 2,3-dihydro-1′-[2-carbonyl-1-methylethyl]spiro[1H-indene-1,4′-piperidine]hydrochloride (24.6 mg, 0.079 mmol) instead of 2,3-dihydro-1′-[2-(carboxy)ethyl]spiro[1H-indene-1,4′-piperidine]hydrochloride. 25.9 mg (87%) of free base was obtained as yellow oil.

This was converted to citric acid salt similar to that described in Example 34 to give 29.5 mg of citrate as a white solid.

¹H NMR (300 MHz, CDCl3) δ 8.27-8.24 (1H, m), 7.23-7.13 (6H, m), 7.05-6.99 (1H, m), 4.15-4.09 (2H, m), 3.42 (1H, br), 3.24-3.19 (2H, m), 2.93-2.85 (5H, m), 2.54-2.38 (3H, m), 2.04-1.92 (4H, m), 1.61-1.56 (2H, m), 1.23 (3H, d, J=6.6 Hz)

MS (ESI positive) m/z: 375(M+H)⁺

IR(KBr):2943, 1728, 1655, 1595, 1483, 1427, 758 cm⁻¹

Anal. Calcd for C25H30N2O—C6H8O7-1.2H2O: C, 63.29; H. 6.92; N, 4.76. Found: C, 63.25; H, 6.95; N, 4.65.

### Preparation 36

### 1-(3-{1-tert-Butyl(dimethyl)silyl]oxy}propyl)-3,4-dihydro-2(1H)-quinolinone

To a stirred solution of NaH [326.0 mg, 8.15 mmol, 60% oil dispersion in mineral oil, which was removed by washing with n-hexane (5 ml×2) before use] and 3,4-dihydro-2(1H)-quinolinone (1.00 g, 6.79 mmol) in DMF (140 ml) was added a solution of (3-bromopropoxy)-tert-butyldimethylsilane (3.1 ml, 13.6 mmol) in DMF (20 ml) at 0° C. The reaction mixture was stirred at 0° C. to room temperature for 3 h. The reaction mixture was cooled to 0° C. and NaHCO3 solution was added to the reaction mixture, then extracted with AcOEt (100 ml×3). The extracts combined were washed with H2O, dried (Na2SO4), and filtered. The filtrate was evaporated in vacuo to afford 2.96 g of crude product, which was purified by silica gel column chromatography (n-Hexane/AcOEt: 4/1 as eluent) to give 1.97 g (91%) of the title compound as pale yellow oil.

¹H NMR (300 MHz, CDCl3) δ 7.26-7.14 (3H, m), 7.02-6.97 (1H, m), 4.05-4.00 (2H, m), 3.71 (2H, t, J=5.9 Hz), 2.91-2.86 (2H, m), 2.66-2.61 (2H, m), 1.94-1.85 (2H, m), 0.93 (9H, s), 0.072 (6H, s)

### Preparation 37

### 1-(3-Hydroxypropyl)-3,4-dihydro-2(1H)-quinolinone

To a stirred solution of 1-(3-{[tert-butyl(dimethyl)silyl]oxy}propyl)-3,4-dihydro-2(1H)-quinolinone (1.97 g, 6.18 mmol) in THF (50 ml) was added tetrabutylammonium fluoride (12.4 ml, 12.36 mmol; 1M solution in THE) at 0° C. After 1 h stirring at room temperature, H2O was added to the reaction mixture, then extracted with AcOEt (50 ml×3). The extracts combined were dried (Na2SO4) and filtered. The filtrate was evaporated in vacuo to afford 2.08 g of crude product, which was purified by silica gel column chromatography (n-Hexane/AcOEt: 1/1 to 0/1as eluent) to give 1.33 g (quant.) of the title compound as pale brown oil.

¹H NMR (300 MHz, CDCl3) δ 7.29-7.17 (2H, m), 7.10-7.00 (2H, m), 4.164.08 (2H, m), 3.57-3.55 (2H, m), 3.36 (1H, m), 2.96-2.90 (2H, m), 2.73-2.67 (2H, m), 1.93-1.84 (2H, m)

### Preparation 38

### 1-(3-Bromopropyl)-3,4-dihydro-2(1H)-quinolinone

To a stirred solution of 1-(3-hydroxypropyl)-3,4-dihydro-2(1B)-quinolinone (100.0 mg, 0.487 mmol) in CH2Cl2 (5 ml) was added triphenylphosphine (153.2 mg, 0.584 mmol) and carbon tetrabromide (242.4 mg, 0.731 mmol) at 0° C. After 1.5 h stirring at room temperature, the reaction mixture was diluted with saturated aqueous NaHCO3 solution, and extracted with CH2Cl2 (15 ml×3), dried (Na2SO4) and filtered. The filtrate was evaporated in vacuo to afford 457.8 mg of crude product, which was purified by silica gel column chromatography (n-Hexane/AcOEt: 3/1 to 1/1as eluent) to give 113.6 mg (87%) of the title compound as colorless oil.

¹H NMR (300 MHz, CDCl3) δ 7.29-7.24 (1H, m), 7.19-7.16 (1H, m), 7.09-6.99 (2H, m), 4.11-4.06 (2H, m), 3.48 (2H, t, J=6.4 Hz), 2.92-2.88 (2H, m), 2.67-2.62 (2H, m), 2.28-2.19 (2H, m)

### Example 92

### 1′-[3-(2-Oxo-3,4-dihydro-[(2H)-quinolinyl)propyl]spiro[isobenzofuran-1(3H),4′-piperidine]citrate

A mixture of spiro[isobenzofuran-[(3H),4′-piperidine]hydrochloride [79.7 mg, 0.353 mmol, this was prepared according to known procedure: Hirokazu Kubota et. al. Chem. Pharm. Bull., 1998, 46, 351], 1-(3-bromopropyl)-3,4-dihydro-2(1H)-quinolinone (113.6 mg, 0.424 mmol), K2CO3 (146.4 mg, 1.059 mmol), and KI (29.4 mg, 0.177 mmol) in MeCN (10 ml) was refluxed with stirring for 16 h. After cooling down to room temperatute, water (30 ml) was added to the reaction mixture and extracted with CH2Cl2 (20 ml×3). The extracts combined were dried (Na2SO4), filtered, and concentrated to give 161.8 mg of crude product. This was purified by silica gel column chromatography (CH2Cl2/MeOH: 20/1 as an eluent). Then extracted product was purified again by preparative TLC (1 mm thick plate, CH2Cl2/MeOH:1511) to afford 74.3 mg (56%) of free base as colorless oil.

¹H NMR (270 MHz, CDCl3) δ 7.30-7.09 (7H, m), 7.03-6.97 (1H, m), 5.06 (2H, s), 4.05-4.00 (2H, m), 2.92-2.87 (4H, m), 2.67-2.62 (2H, m), 2.55-2.38 (4H, m), 2.06-1.86 (4H, m), 1.80-1.76 (2H, m)

This was converted to citric acid salt similar to that described in Example 34 to give 103.3 mg of citrate as a white solid.

MS (ESI positive) m/z: 377(M+H)⁺

IR(KBr): 1387, 1188, 1045, 760 cm⁻¹

Anal. Calcd for C24H28N2O2-C6H8O7-1.2H2O-0.17C6H14-0.25CH2Cl2: C, 60.03; H, 6.60; N, 4.47. Found: C, 59.97; H, 6.36; N, 4.46

### Example 93

### 1′-[3-(2-Oxo-3,4-dihydro-[(2H)-quinolinyl)propyl]spiro[1H-indene-1,4′-piperidine]citrate

This was prepared according to the procedure described in Example 92 using spiro[1H-indene-1,4′-piperidine]hydrochloride (This was prepared according to known procedure:M. S. Chambers et al, J. Med. Chem. 1992, 35, 2033) instead of spiro[isobenzofuran-[(3H),4′-piperidine]hydrochloride. 61.4 mg (46%) of free base was obtained as pale yellow oil.

¹HNMR (270 MHz, CDCl3) δ 7.39-7.12 (7H, m), 7.04-6.98 (1H, m), 6.84 (1H, d, J=5.6 Hz), 6.74 (1H, d, J=5.6 Hz), 4.07-4.02 (2H, m), 3.06-3.02 (2H, m), 2.93-2.88 (2H, m), 2.68-2.56 (4H, m), 2.42-2.34 (2H, m), 2.27-2.22 (2H, m), 1.98-1.93 (2H, m), 1.40-1.35 (2H, m)

This was converted to citric acid salt similar to that described in Example 34 to give 83.0 mg of citrate as a pale yellow solid.

MS (ESI positive) m/z: 373(M+H)⁺

IR(KBr):2953, 1732, 1186, 756 cm⁻¹

Anal. Calcd for C25H28N2O—C6H8O7-3H2O: C, 62.93; H, 6.64; N, 4.73. Found: C, 62.65; H, 6.53; N, 4.36

### Example 94

### 1-Methyl-1′-3-(2-oxo-3,4-dihydro-1-(R)-quinolinyl)propyl]spiro[indoline-3,4′-piperidine]citrate

This was prepared according to the procedure described in Example 92 using 1-methylspiro(indoline-3,4′-piperidine) [51.5 mg, 0.255 mmol, this was prepared according to known procedure:Efange, Simon M. N. et al, J. Med. Chem. 1997, 40, 3905] instead of spiro[isobenzofuran-[(3H),4′-piperidine]hydrochloride. 48.8 mg (49%) of free base was obtained as pale yellow oil.

¹H NMR (270 MHz, CDCl3) δ 7.27-6.97 (6H, m), 6.72-6.67 (1H, m), 6.49-6.46 (1H, m), 4.04-3.98 (2H, m), 3.19 (2H, s), 2.92-2.87 (4H, m), 2.76 (3H, s), 2.67-2.62 (2H, m), 2.50-2.45 (2H, m), 2.17-2.08 (2H, m), 2.00-1.84 (4H, m), 1.75-1.71 (2H, m)

This was converted to citric acid salt similar to that described in Example 34 to give 67.5 mg of citrate as a pale yellow solid.

MS (ESI positive) m/z: 390(M+H)⁺

IR(KBr):2951, 1717, 1387, 1192, 756 cm⁻¹

Anal. Calcd for C25H31N3O—C6H8O7-0.8H2O-0.1C6H14-0.2CH2Cl2: C, 61.52; H. 6.75; N, 6.77. Found: C, 61.52; H, 6.90; N, 6.39

### Preparation 39

### 1′-(3-Hydroxypropyl)spiro[1H-indene-1,4′-piperidine]

This was prepared according to the procedure described in Preparation 6 using spiro[1H-indene-1,4′-piperidine]hydrochloride instead of 2,3-dihydrospiro[1H-indene-1,4′-piperidine]hydrochloride. 1.8 g (55%) of the title product was obtained as a white solid.

¹H NMR (270 MHz, CDCl3) δ 7.40-7.15 (4H, m), 6.82 (1H, d, J=5.6 Hz), 6.75 (1H, d, J=5.6 Hz), 3.87 (2H, t, J=5.3 Hz), 3.25-3.10 (2H, m), 2.75 (2H, t, J=5.8 Hz), 2.45-2.30 (2H, m), 2.23-2.05 (2H, m), 1.86-1.72 (2H, m), 1.45-1.35 (2H, m).

### Preparation 40

### 1′-(3-Mesyloxypropyl)spiro[1H-indene-1,4′-piperidine]

This was prepared according to the procedure described in Preparation 7 using 1′-(3-hydroxypropyl)spiro[1H-indene-1,4′-piperidine] instead of 2,3-dihydro-1′-(3-hydroxypropyl)spiro[1H-indene-1,4′-piperidine]. 158 mg (quant) of the title product was obtained as colorless oil.

¹H NMR (270 MHz, CDCl3) δ 7.45-7.15 (4H, m), 6.83 (1H, d, J=5.6 Hz), 6.74 (1H, d, J=5.6 Hz), 4.35 (2H, t, J=6.4 Hz), 3.03 (3H, s), 3.02-2.92 (2H, m), 2.59 (2H, t, J=7.1 Hz), 2.42-2.29 (2H, m), 2.23-2.09 (2H, m), 2.07-1.94 (2H, m), 1.42-1.30 (2H, m).

### Preparation 41

### 1′-[3-[3-(Hydroxymethyl)-2-oxo-[(2H)quinolinyl]propyl]spiro[1H-indene-1,4′-piperidine]

This was prepared according to the procedure described in Example 4 using 1′-(3-mesyloxypropyl)spiro[1H-indene-1,4′-piperidine] and 3-hydroxymethyl-2(1H)-quinolinone (this was prepared according to known procedure: M. Uchida et al, Chem. Pharm. Bull. 1985, 33, 3775) instead of 2,3-dihydro-1′-(3-mesyloxypropyl)spiro[1H-indene-1,4′-piperidine] and benzothiazol-2-one. 91 mg (58%) of the title product was obtained as a pale brown amorphous.

¹H NMR (270 MHz, CDCl3) δ 7.64-7.54 (4H, m), 7.42-7.18 (5H, m), 6.85 (1H, d, 3=5.6 Hz), 6.75 (1H, d, J=5.6 Hz), 4.69 (2H, s), 4.50-4.40 (2H, m), 3.10-2.98 (2H, m), 2.64 (2H, t, J=6.9 Hz), 2.44-2.32 (2H, m), 2.27-2.12 (2H, m), 2.10-1.98 (2H, m), 1.44-1.33 (2H, m).

MS (ESI positive) m/z: 401 (M+H)⁺.

### Example 95

### 1′-[3-[3-(Hydroxymethyl)-2-oxo-3,4-dihydro-[(2H)-quinolinyl]propyl]spiro[1H-indene-1,4′-piperidine]citrate

To a stirred solution of 1′-[3-[3-(Hydroxymethyl)-2-oxo-[(2H)-quinolinyl]propyl]spiro[1H-indene-1,4′-piperidine] (90 mg, 0.23 mmol) in toluene (4 ml) was added L-selectride (1.0M THF solution, 0.67 ml) at −78° C. The resulting reaction mixture was warmed to −30° C., and stirred for 2 h. L-selectride (11.0M THF solution, 0.67 ml) was added to this mixture at −30° C., and the reaction mixture warmed to 0° C. After 1 h, this was quenched with aqueous NaHCO3 solution and extracted with CH2Cl2. The extracts combined were dried (MgSO4), filtered, and concentrated. The resulting residue was purified by preparative TLC (1 mm thick silica gel plate: CH2Cl2/MeOH:20/1) to afford 36 mg (40%) of free base as a colorless amorphous.

¹H NMR (270 MHz, CDCl3) δ 7.40-7.12 (7H, m), 7.08-6.98 (1H, m), 6.84 (1H, d, 3=5.6 Hz), 6.74 (1H, d, J=5.6 Hz), 4.10-4.00 (2H, m), 3.89 (2H, d, J=5.3 Hz), 3.08-2.66 (5H, m), 2.55 (2H, t, J=7.4 Hz), 2.42-2.28 (2H, m), 2.27-2.12 (2H, m), 2.08-1.80 (2H, m), 1.44-1.32 (2H, m).

This was converted to citrate salt similar to that described in Example 34 to give 67.5 mg of the title product as a white amorphous solid.

MS (ESI positive) m/z: 403 (M+H)⁺

IR(KBr): 3358, 2943, 1728, 1651, 1601, 1464, 1394, 1186, 756 cm⁻¹

Anal. Calcd for C26H30N2O2-C6H8O7-1.78H2O: C, 61.33; H, 6.68; N, 4.47. Found: C, 60.96; H, 6.28; N, 4.28

### Example 96

### 2,3-Dihydro-1-[3-(6-fluoro-2,3-dihydro-1-H-indol-1-yl)-3-oxopropyl]spiro[1H-indene1,4′-piperidine]formate

To 6-fluoro-2,3-dihydro-1H-indole (75 μmol) was added the mixture of 2,3-Dihydro-1′-[2-(carboxy)ethyl]spiro[1H-indene-1,4′-piperidine]hydrochloride (50 μmol, this was prepared in Preparation 2) and iPrNEt (125 μmol) dissolved in DCE (500 μl). HBTU (60 μmol) dissolved in DCE/DMF (200 μl/300 μl) was added, then the reaction mixture was stirred at r.t. for 24 h. To this mixture was added phenylisocyanate (9 mg, 75 μmol), and the resulting mixture was stirred at rt for 1 h. The mixture was loaded onto a BondElute SCX cartridge (500 mg/3 ml) preconditioned 1 ml of MeOH. The solid-phase matrix was washed twice with 10 ml of MeOH/DCM (3/1) and then eluted with 2 ml of 1M ammonia/MeOH. The eluate was concentrated to dryness by N₂ gas blow and vacuum centrifuge, providing crude product, which was purified with preparative LS/MS to give 1.5 mg (7%) of the title product as the formate form.

MS (ESI positive) m/z: 379 (M+H)⁺

HPLC purity (UV210-400 nm): >99%

1-13. (Cancelled).

14. A compound of the following formula: 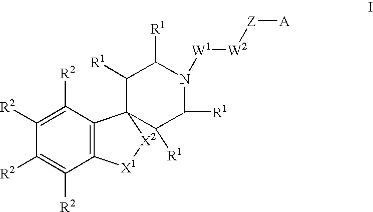
or pharmaceutically accptable salts thereof, wherein
each R¹ is independently selected from hydrogen and (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; or
two R¹ groups taken together form —CH2— or —(CH2)₂— and the remaining R¹ groups are defined as above;
each R² is independently selected from
hydrogen; halo; hydroxy; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a1}R N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—;
(C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—;
aryl selected from phenyl and naphthyl; and four- to eight-membered heterocyclyl containing one to four hetero atoms in the ring independently selected from nitrogen, oxygen and sulfur;
X¹ and X² are each CH₂; or
X¹ and X² taken together form CH═CH;
W¹ and W² are independently selected from CR^{W1}R^{W2}, wherein
R^{W1} and R^{W2} are independently selected from hydrogen; halo; hydroxy; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2} N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—;
C(═O)-[(C₁-C₆)alkyl] wherein said (C₁-C₆)alkyl is optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; C(═O)—NR^{W11}R^{W12} wherein R^{W11} and R^{W12} are independently selected from hydrogen and (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a12}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; NR^{W13}R^{W14} wherein R^{W13} and R^{W14} are independently selected from hydrogen and (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2} R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; aryl selected from phenyl and naphthyl; and four- to eight-membered heterocyclyl containing one to four hetero atoms independently selected from nitrogen, oxygen and sulfur;
A is selected from AA; AB; AC and AE: 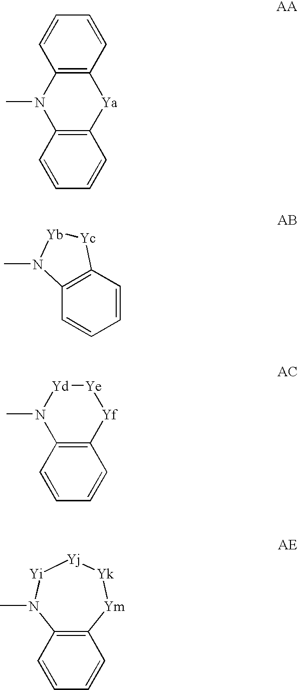
wherein
Y^{a} is selected from (CH2)ₙ₂ wherein n2 is an integer selected from 0, 1 and 2; C(═O); NH; O and S;
Y^{b}, Y^{c}, Y^{d}, Y^{e}, Y^{f}, Yⁱ, Y^{j}, Y^{k} and Yₘ are independently selected from C(═O); CR^{Y1}R^{Y2}; CR^{Y3}[C(═O)R^{Y4}]; CR^{Y3}[NR^{Y5}C(═O)R^{Y4}]; CR^{Y3}[C(═O)NR^{Y6} R^{Y7}]; CR^{Y3}NR^{Y6} R^{Y7]}; O; S; SO₂; NH; N[(C₁-C₆)alkyl] wherein said (C₁-C₆)alkyl is optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; N—(CH₂)ₙ₃-heterocyclyl wherein n3 is an integer selected from 0, 1, 2 and 3, and said heterocyclyl contains from four to eight ring atoms one or two of which are independently selected from nitrogen, oxygen and sulfur; N—(CH₂)ₙ₄-aryl wherein n4 is an integer selected from 0, 1, 2 and 3, and said aryl is selected from phenyl and naphthyl; and N—(CH₂)ₙ₅-heteroaryl wherein n5 is an integer selected from 0, 1, 2 and 3, and said heteroaryl is a five to ten membered aromatic heterocyclyl containing from one to four hetero atoms independently selected from nitrogen, oxygen and sulfur; or
Y^{b} and Y^{c} taken together form a group selected from CR^{Y81}═CR^{Y82}; CR^{Y83}═N and N═N; and Y^{d}, Yₑ and Y^{f} are defined as above; wherein
R^{Y1}, R^{Y2} and R^{Y5} are independently selected from hydrogen; hydroxy; non-, mono- and di-substituted amino wherein the substituents are independently selected from (C₁-C₆)alkyl; [(C₁-C₆)alkyl]-C(═O)—; [(C₁-C₆)alkoxy]-C(═O)—; [(C₁-C₆)alkyl]-SO₂—; and four- to eight-membered heterocyclyl containing one to four hetero atoms independently selected from nitrogen, oxygen and sulfur, wherein said heterocyclyl is optionally substituted with one to three substituents independently selected from hydroxy, (C₁-C₆)alkyl, NH₂—C(O═)—, [(C₁-C₆)alkyl]-NH—C(═O)—, [(C₁-C₆)alkyl]₂-N—C(═O)—, and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7} R^{a8} N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; or
R^{Y1} and R^{Y2} taken together with the carbon atom to which they are attached form spiropyrrolidinyl or spiropiperidinyl, both of which are optionally N-substituted with a substituent selected from (C₁-C₆)alkyl, (C₁-C₆)alkyl-C(═O)—, [(C₁-C₆)alkyl]-C(═O)-(C₁-C₆)alkyl and aryl-(C═O)— wherein aryl is selected from phenyl and naphthyl; and R^{Y5} is defined as above;
R^{Y3} is hydrogen;
R^{Y4} is selected from hydroxy; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and
R^{Y6} and R^{Y7} are independently selected from hydrogen; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; hetrocyclyl-(CH₂)ₙ₆— wherein n6 is an integer selected from 0, 1, 2, 3 and 4 and said heterocyclyl is four to eight membered containing one to three hetero atoms independently selected from nitrogen, oxygen and sulfur, wherein said heterocyclyl is optionally substituted with one to three substituents independently selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; and hetroaryl-(CH₂)ₙ₇— wherein n7 is an integer selected from 0, 1, 2, 3 and 4 and said heteroaryl is five to ten membered containing one to three hetero atoms independently selected from nitrogen, oxygen and sulfur, wherein said heteroaryl is optionally substituted with one to three substituents independently selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; or
R^{Y6} and R^{Y7} taken together with the nitrogen atom to which they are attached form a four to eight heterocyclyl optionally containing, in addition to the nitrogen atom, one to two additional hetero atoms independently selected from nitrogen, oxygen and sulfur, and said heterocyclyl is optionally substituted with one substituent selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—;
R^{Y81}, R^{Y82} and R^{Y83} are independently selected from R^{Y811} and R^{Y812}C(═O)— wherein R^{Y811} and R^{Y812} are independently selected from hydrogen; hydroxy; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and
said A is optionally substituted in the fused benzene rings with one to four substituents independently selected from halo; hydroxy; mercapto; phenyl; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁ —C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and
Z is selected from C(═O); (CH₂)ₙ₈ wherein n8 is an integer selected from 0, 1 and 2; and CHR^{Z1} wherein R^{Z1} is selected from carboxy; (C₁-C₆)alkoxy-C(═O)—; non-, mono- and di-substituted amino wherein the substituents are independently selected from (C₁-C₆)alkyl, [(C₁-C₆)alkyl]-C(═O)—, [(C₁-C₆)alkyl]-C(═O)—O— and [(C₁-C₆)alkyl]-SO₂—; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and [C(═O)—NR^{Z11}R^{Z12}] wherein R^{Z11} and R^{Z12} are independently selected from hydrogen and (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—.

15. A compound according to claim 1 wherein
all R¹ are hydrogen
each R² is independently selected from hydrogen and halo;
X¹ and X² are each CH₂; or
X¹ and X² taken together form CH═CH;
W¹ and W² are independently selected from CR^{W1}R^{W2}, wherein R^{W1} and R^{W2} are independently selected from hydrogen; halo; hydroxy; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; C(═O)-[(C₁-C₆)alkyl] wherein said (C₁-C₆)alkyl is optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; C(═O)—NR^{W11}R^{W12} wherein R^{W11} and R^{W12} are independently selected from hydrogen and (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; NR^{W3}R^{W14} wherein R^{W13} and R^{W14} are independently selected from hydrogen and (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; aryl selected from phenyl and naphthyl; and four- to eight-membered heterocyclyl containing one to four hetero atoms independently selected from nitrogen, oxygen and sulfur;
A is AB wherein
Y^{b} and Y^{c} are independently selected from C(═O); CR^{Y1}R^{Y2}; CR^{Y3}[C(═O)R^{Y4}]; CR^{Y3}[C(═O)NR^{Y6} R^{Y7}]; CR^{Y3}[NR^{Y6} R^{Y7}]; O; S; SO₂; NH; N[(C₁-C₆)alkyl] wherein said (C₁-C₆)alkyl is optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a1}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; N—(CH₂)ₙ₃-heterocyclyl wherein n3 is an integer selected from 0, 1, 2 and 3, and said heterocyclyl contains from four to eight ring atoms one or two of which are independently selected from nitrogen, oxygen and sulfur; N—(CH₂)ₙ₄-aryl wherein n4 is an integer selected from 0, 1, 2 and 3, and said aryl is selected from phenyl and naphthyl; and N—(CH₂)ₙ₅-heteroaryl wherein n5 is an integer selected from 0, 1, 2 and 3, and said heteroaryl is a five to ten membered aromatic heterocyclyl containing from one to four hetero atoms independently selected from nitrogen, oxygen and sulfur; or
Y^{b} and Y^{c} taken together form a group selected from CR^{Y81}═CR^{Y82}; CR^{Y83}═N and N═N; and Y^{d}, Y^{e}, Y^{f}, Y^{g} and Y^{h} are defined as above;
R^{Y1} and R^{Y2} are independently selected from hydrogen; hydroxy; non-, mono- and di-substituted amino wherein the substituents are independently selected from (C₁-C₆)alkyl; [(C₁-C₆)alkyl]-C(═O)—; [(C₁-C₆)alkoxy]-C(═O)—; [(C₁-C₆)alkyl]-SO₂—; and four- to eight-membered heterocyclyl containing one to four hetero atoms independently selected from nitrogen, oxygen and sulfur, wherein said heterocyclyl is optionally substituted with one to three substituents independently selected from hydroxy, (C₁-C₆)alkyl, NH₂—C(O═)—, [(C₁-C₆)alkyl]-NH—C(═O)—, [(C₁-C₆)alkyl]₂-N—C(═O)—, and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6} N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; or
R^{Y1} and R^{Y2} taken together with the carbon atom to which they are attached form spiropyrrolidinyl or spiropiperidinyl, both of which are optionally N-substituted with a substituent selected from (C₁-C₆)alkyl, (C₁-C₆)alkyl-C(═O)—, [(C₁-C₆)alkyl]-C(═O)-(C₁-C₆)alkyl and aryl-(C═O)— wherein aryl is selected from phenyl and naphthyl;
R^{Y3} is hydrogen;
R^{Y4} is selected from hydroxy; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5} R^{a6} N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and
R^{Y5}, R^{Y6} and R^{Y7} are independently selected from hydrogen; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; hetrocyclyl-(CH₂)ₙ₆— wherein n6 is an integer selected from 0, 1, 2, 3 and 4 and said heterocyclyl is four to eight membered containing one to three hetero atoms independently selected from nitrogen, oxygen and sulfur, wherein said heterocyclyl is optionally substituted with one to three substituents independently selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; and hetroaryl-(CH₂)ₙ₇— wherein n7 is an integer selected from 0, 1, 2, 3 and 4 and said heteroaryl is five to ten membered containing one to three hetero atoms independently selected from nitrogen, oxygen and sulfur, wherein said heteroaryl is optionally substituted with one to three substituents independently selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; or
R^{Y6} and R^{Y7} taken together with the nitrogen atom to which they are attached form a four to eight heterocyclyl optionally containing, in addition to the nitrogen atom, one to two additional hetero atoms independently selected from nitrogen, oxygen and sulfur, and said heterocyclyl is optionally substituted with one substituent selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—;
R^{Y81}, R^{Y82} and R^{Y83} are independently selected from R^{Y811} and R^{Y812}C(═O)— wherein R^{Y81} and R^{Y812} are independently selected from hydrogen; hydroxy; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{z1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁ —C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and
said A is optionally substituted in the fused benzene rings with one to four substituents independently selected from halo; hydroxy; mercapto; phenyl; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and
Z is selected from C(═O); (CH₂)ₙ₈ wherein n8 is an integer selected from 0, 1 and 2; and CHR^{Z1} wherein
R^{Z1} is selected from carboxy; (C₁-C₆)alkoxy-C(═O)—; non-, mono- and di-substituted amino wherein the substituents are independently selected from (C₁-C₆)alkyl, [(C₁-C₆)alkyl]-C(═O)—, [(C₁-C₆)alkyl]-C(═O)—O— and [(C₁-C₆)alkyl]-SO₂—; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and
[C(═O)—NR^{Z11}R^{Z12}] wherein R^{Z11 and R}^{Z12} are independently selected from hydrogen and (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—.

16. A compound according to claim 2 wherein
all R¹ are hydrogen
each R² is independently selected from hydrogen and halo;
X¹ and X² are each CH₂; or
X¹ and X² are taken together form CH═CH;
W¹ and W² are both CH₂;
A is AB wherein
both Y^{b} and Y^{c} are independently selected from C(═O); CR^{Y1}R^{Y2}; CR^{Y3}[C(═O)R^{Y4}]; CR^{Y3}[C(═O)NR^{Y6}R^{Y7}]; and CR^{Y3}[NR^{Y6}R^{Y7}], wherein
R^{Y1} and R^{Y2} are independently selected from hydrogen; hydroxy; non-, mono- and di-substituted amino wherein the substituents are independently selected from (C₁-C₆)alkyl; [(C₁-C₆)alkyl]-C(═O)—; [(C₁-C₆)alkoxy]-C(═O)—; [(C₁-C₆)alkyl]-SO₂—; and
four- to eight-membered heterocyclyl containing one to four hetero atoms independently selected from nitrogen, oxygen and sulfur, wherein said heterocyclyl is optionally substituted with one to three substituents independently selected from hydroxy, (C₁-C₆)alkyl, NH₂—C(O═)—, [(C₁-C₆)alkyl]-NH—C(═O)—, [(C₁-C₆)alkyl]₂-N—C(═O)—, and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; or
R^{Y1} and R^{Y2} taken together with the carbon atom to which they are attached form spiropyrrolidinyl or spiropiperidinyl, both of which are optionally N-substituted with a substituent selected from (C₁-C₆)alkyl, (C₁-C₆)alkyl-C(═O)—, [(C₁-C₆)alkyl]-C(═O)-(C₁-C₆)alkyl and aryl-(C═O)— wherein aryl is selected from phenyl and naphthyl;
R^{Y3} is hydrogen;
R^{Y4} is selected from hydroxy; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and
R^{Y6} and R^{Y7} are independently selected from hydrogen; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3} R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; hetrocyclyl-(CH₂)ₙ₆— wherein n6 is an integer selected from 0, 1, 2, 3 and 4 and said heterocyclyl is four to eight membered containing one to three hetero atoms independently selected from nitrogen, oxygen and sulfur, wherein said heterocyclyl is optionally substituted with one to three substituents independently selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; and hetroaryl-(CH₂)ₙ₇— wherein n7 is an integer selected from 0, 1, 2, 3 and 4 and said heteroaryl is five to ten membered containing one to three hetero atoms independently selected from nitrogen, oxygen and sulfur, wherein said heteroaryl is optionally substituted with one to three substituents independently selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; or
R^{Y6} and R^{Y7} taken together with the nitrogen atom to which they are attached form a four to eight heterocyclyl optionally containing, in addition to the nitrogen atom, one to two additional hetero atoms independently selected from nitrogen, oxygen and sulfur, and said heterocyclyl is optionally substituted with one substituent selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—;
said A is optionally substituted in the fused benzene rings with one to four substituents independently selected from halo; hydroxy; mercapto; phenyl; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a6}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and
Z is selected from C(═O); (CH₂)ₙ₈ wherein n8 is an integer selected from 0, 1 and 2; and CHR^{Z1} wherein
R^{Z1} is selected from carboxy; (C₁-C₆)alkoxy-C(═O)—; non-, mono- and di-substituted amino wherein the substituents are independently selected from (C₁-C₆)alkyl, [(C₁-C₆)alkyl]-C(═O)—, [(C₁-C₆)alkyl]-C(═O)—O— and [(C₁-C₆)alkyl]-SO₂—; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and [C(═O)—NR^{Z11}R^{Z12}] wherein R^{Z11} and R^{Z12} are independently selected from hydrogen and (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—.

17. A compound according to claim 3 wherein
all R¹ are hydrogen
each R² is independently selected from hydrogen and halo;
X¹ is selected from (CH2)n1 wherein n1 is an integer selected from 1, 2 and 3; O; NH; S; C(═O); SO₂; and N[(C₁-C₄)alkyl];
X¹ and X² are each CH₂; or
X¹ and X² are taken together form CH═CH;
W¹ and W² are both CH₂;
A is AB wherein
Y^{b} is CR^{Y3}[C(═O)NR^{Y6}R^{Y7}]; and
Y^{c} is selected from CR^{Y1}R^{Y2}; CR^{Y3} [C(═O)R^{Y4}]; CR^{Y3}[C(═O)NR^{Y6}R^{Y7}]; and CR^{Y3}[NR^{Y6} R^{Y7}], wherein
R^{Y1} and R^{Y2} are independently selected from hydrogen; hydroxy; non-, mono- and di-substituted amino wherein the substituents are independently selected from (C₁-C₆)alkyl; [(C₁-C₆)alkyl]-C(═O)—; [(C₁-C₆)alkoxy]-C(═O)—; [(C₁-C₆)alkyl]-SO₂—; and four- to eight-membered heterocyclyl containing one to four hetero atoms independently selected from nitrogen, oxygen and sulfur, wherein said heterocyclyl is optionally substituted with one to three substituents independently selected from hydroxy, (C₁-C₆)alkyl, NH₂—C(O═)—, [(C₁-C₆)alkyl]-NH—C(═O)—, [(C₁-C₆)alkyl]₂-N—C(═O)—, and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a1}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; or
R^{Y1} and R^{Y2} taken together with the carbon atom to which they are attached form spiropyrrolidinyl or spiropiperidinyl, both of which are optionally N-substituted with a substituent selected from (C₁-C₆)alkyl, (C₁-C₆)alkyl-C(═O)—, [(C₁-C₆)alkyl]-C(═O)-(C₁-C₆)alkyl and aryl-(C═O)— wherein aryl is selected from phenyl and naphthyl;
R^{Y3} is hydrogen;
R^{Y4} is selected from hydroxy; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and
R^{Y5}, R^{Y6} and R^{Y7} are independently selected from hydrogen; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; hetrocyclyl-(CH₂)ₙ₆— wherein n6 is an integer selected from 0, 1, 2, 3 and 4 and said heterocyclyl is four to eight membered containing one to three hetero atoms independently selected from nitrogen, oxygen and sulfur, wherein said heterocyclyl is optionally substituted with one to three substituents independently selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; and hetroaryl-(CH₂)ₙ₇— wherein n7 is an integer selected from 0, 1, 2, 3 and 4 and said heteroaryl is five to ten membered containing one to three hetero atoms independently selected from nitrogen, oxygen and sulfur, wherein said heteroaryl is optionally substituted with one to three substituents independently selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; or
R^{Y6} and R^{Y7} taken together with the nitrogen atom to which they are attached form a four to eight heterocyclyl optionally containing, in addition to the nitrogen atom, one to two additional hetero atoms independently selected from nitrogen, oxygen and sulfur, and said heterocyclyl is optionally substituted with one substituent selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—;
said A is optionally substituted in the fused benzene rings with one to four substituents independently selected from halo; hydroxy; mercapto; phenyl; (C₁-C₆)alkyl optionally substituted with one to three substituents independently selected from halo, hydroxy, carboxy, (C₁-C₆)alkoxy, (C₁-C₆)alkoxy-C(═O)— and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, (C₁-C₆)alkoxy, (C₁-C₆)alkoxy-C(═O)— and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; and
Z is selected from C(═O); (CH₂)ₙ₈ wherein n8 is an integer selected from 0, 1 and 2; and CHR^{Z1} wherein
R^{Z1} is selected from carboxy; (C₁-C₆)alkoxy-C(═O)—; non-, mono- and di-substituted amino wherein the substituents are independently selected from (C₁-C₆)alkyl, [(C₁-C₆)alkyl]-C(═O)—, [(C₁-C₆)alkyl]-C(═O)—O— and [(C₁-C₆)alkyl]-SO₂—; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and [C(═O)—NR^{Z11}R^{Z12}] wherein R^{Z11} and R^{Z12} are independently selected from hydrogen and (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—.

18. A compound according to claim 4 wherein
all R¹ are hydrogen
each R² is independently selected from hydrogen and halo;
X¹ and X² are each CH₂; or
X¹ and X² are taken together form CH═CH;
W¹ and W² are both CH₂;
A is AB wherein
Y^{b} is CR^{Y3}[C(═O)NR^{Y6} R^{Y7}]; and
Y_{c} is selected from CR^{Y1}R^{Y2}; CR^{Y3}[C(═O)R^{Y4}]; CRY [C(═O)NR^{Y6} R^{Y7}]; and CR^{Y3}[NR^{Y6} R^{Y7}]; wherein
R^{Y1} and R^{Y2} are independently selected from hydrogen; hydroxy; non-, mono- and di-substituted amino wherein the substituents are independently selected from (C₁-C₆)alkyl; [(C₁-C₆)alkyl]-C(═O)—; [(C₁-C₆)alkoxy]-C(═O)—; [(C₁-C₆)alkyl]-SO₂—; and four- to eight-membered heterocyclyl containing one to four hetero atoms independently selected from nitrogen, oxygen and sulfur, wherein said heterocyclyl is optionally substituted with one to three substituents independently selected from hydroxy, (C₁-C₆)alkyl, NH₂—C(O═)—, [(C₁-C₆)alkyl]-NH—C(═O)—, [(C₁-C₆)alkyl]₂-N—C(═O)—, and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; or
R^{Y1} and R^{Y2} taken together with the carbon atom to which they are attached form spiropyrrolidinyl or spiropiperidinyl, both of which are optionally N-substituted with a substituent selected from (C₁-C₆)alkyl, (C₁-C₆)alkyl-C(═O)—, [(C₁-C₆)alkyl]-C(═O)-(C₁-C₆)alkyl and aryl-(C═O)— wherein aryl is selected from phenyl and naphthyl;
R^{Y3} is hydrogen;
R^{Y4} is selected from hydroxy; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a1}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and
R^{Y5}, R^{Y6} and R^{Y7} are independently selected from hydrogen; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; hetrocyclyl-(CH₂)ₙ₆— wherein n6 is an integer selected from 0, 1, 2, 3 and 4 and said heterocyclyl is four to eight membered containing one to three hetero atoms independently selected from nitrogen, oxygen and sulfur, wherein said heterocyclyl is optionally substituted with one to three substituents independently selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; and hetroaryl-(CH₂)ₙ₇— wherein n7 is an integer selected from 0, 1, 2, 3 and 4 and said heteroaryl is five to ten membered containing one to three hetero atoms independently selected from nitrogen, oxygen and sulfur, wherein said heteroaryl is optionally substituted with one to three substituents independently selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; or
R^{Y6} and R^{Y7} taken together with the nitrogen atom to which they are attached form a four to eight heterocyclyl optionally containing, in addition to the nitrogen atom, one to two additional hetero atoms independently selected from nitrogen, oxygen and sulfur, and said heterocyclyl is optionally substituted with one substituent selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—;
said A is optionally substituted in the fused benzene rings with one to four substituents independently selected from halo; hydroxy; mercapto; phenyl; (C₁-C₆)alkyl optionally substituted with one to three substituents independently selected from halo, hydroxy, carboxy, (C₁-C₆)alkoxy, (C₁-C₆)alkoxy-C(═O)— and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, (C₁-C₆)alkoxy, (C₁-C₆)alkoxy-C(═O)— and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; and
Z is C(═O).

19. A compound according to claim 3 wherein
all R¹ are hydrogen
each R² is independently selected from hydrogen and halo;
X¹ and X² are each CH₂; or
X¹ and X² are taken together form CH═CH;
W¹ and W² are both CH₂;
A is AB wherein
Y^{b} is CR^{Y1}R^{Y2}; and
Y^{c} is selected from CR^{Y1}R^{Y2}; CR^{Y2}; [C(═O)R^{Y4}]; CR^{Y3}[C(═O)NR^{Y6}R^{Y7}]; and CR^{Y3}[NR^{Y6}R^{Y7}]; or
Y^{b} and Y^{c} taken together form a group selected from CH2—CH2 and CH2═CH2;
R^{Y1} and R^{Y2} are independently selected from hydrogen; hydroxy; non-, mono- and di-substituted amino wherein the substituents are independently selected from (C₁-C₆)alkyl; [(C₁-C₆)alkyl]-C(═O)—; [(C₁-C₆)alkoxy]-C(═O)—; [(C₁-C₆)alkyl]-SO₂—; and four- to eight-membered heterocyclyl containing one to four hetero atoms independently selected from nitrogen, oxygen and sulfur, wherein said heterocyclyl is optionally substituted with one to three substituents independently selected from hydroxy, (C₁-C₆)alkyl, NH₂—C(O═)—, [(C₁-C₆)alkyl]-NH—C(═O)—, [(C₁-C₆)alkyl]₂-N—C(═O)—, and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a1} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a1}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; or
R^{Y1} and R^{Y2} taken together with the carbon atom to which they are attached form spiropyrrolidinyl or spiropiperidinyl, both of which are optionally N-substituted with a substituent selected from (C₁-C₆)alkyl, (C₁-C₆)alkyl-C(═O)—, [(C₁-C₆)alkyl]-C(═O)-(C₁ —C₆)alkyl and aryl-(C═O)— wherein aryl is selected from phenyl and naphthyl;
R^{Y3} is hydrogen;
R^{Y4} is selected from hydroxy; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C¹-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6} R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and
R^{Y6} and R^{Y7} are independently selected from hydrogen; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; hetrocyclyl-(CH₂)ₙ₆— wherein n6 is an integer selected from 0, 1, 2, 3 and 4 and said heterocyclyl is four to eight membered containing one to three hetero atoms independently selected from nitrogen, oxygen and sulfur, wherein said heterocyclyl is optionally substituted with one to three substituents independently selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; and hetroaryl-(CH₂)ₙ₇— wherein n7 is an integer selected from 0, 1, 2, 3 and 4 and said heteroaryl is five to ten membered containing one to three hetero atoms independently selected from nitrogen, oxygen and sulfur, wherein said heteroaryl is optionally substituted with one to three substituents independently selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; or
R^{Y6} and R^{Y7} taken together with the nitrogen atom to which they are attached form a four to eight heterocyclyl optionally containing, in addition to the nitrogen atom, one to two additional hetero atoms independently selected from nitrogen, oxygen and sulfur, and said heterocyclyl is optionally substituted with one substituent selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—;
said A is optionally substituted in the fused benzene rings with one to four substituents independently selected from halo; hydroxy; mercapto; phenyl; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and
Z is C(═O).

20. A compound according to claim 2 wherein
all R¹ are hydrogen
each R² is independently selected from hydrogen and halo;
X¹ and X² are each CH₂; or
X¹ and X² are taken together form CH═CH;
W¹ and W² are both CH₂;
A is AB wherein
Y^{b} is selected from C(═O); CR^{Y1}R^{Y2}; CR^{Y3} [C(═O)R^{Y4}]; CR^{Y3}[NR^{Y5}C(═O)R^{Y4}]; CR^{Y3}[C(═O)NR^{Y6}R^{Y7}]; and CR^{Y3}[NR^{Y6} R^{Y7}];
Y^{c} is selected from O; S; SO₂; NH; N[(C₁-C₆)alkyl] wherein said (C₁-C₆)alkyl is optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; N—(CH₂)ₙ₃-heterocyclyl wherein n3 is an integer selected from 0, 1, 2 and 3, and said heterocyclyl contains from four to eight ring atoms one or two of which are independently selected from nitrogen, oxygen and sulfur; N—(CH₂)ₙ₄-aryl wherein n4 is an integer selected from 0, 1, 2 and 3, and said aryl is selected from phenyl and naphthyl; and N—(CH₂)ₙ₅-heteroaryl wherein n5 is an integer selected from 0, 1, 2 and 3, and said heteroaryl is a five to ten membered aromatic heterocyclyl containing from one to four hetero atoms independently selected from nitrogen, oxygen and sulfur; wherein
R^{Y1} and R^{Y2} are independently selected from hydrogen; hydroxy; non-, mono- and di-substituted amino wherein the substituents are independently selected from (C₁-C₆)alkyl; [(C₁-C₆)alkyl]-C(═O)—; [(C₁-C₆)alkoxy]-C(═O)—; [(C₁-C₆)alkyl]-SO₂—; and four- to eight-membered heterocyclyl containing one to four hetero atoms independently selected from nitrogen, oxygen and sulfur, wherein said heterocyclyl is optionally substituted with one to three substituents independently selected from hydroxy, (C₁-C₆)alkyl, NH₂—C(O═)—, [(C₁-C₆)alkyl]-NH—C(═O)—, [(C₁-C₆)alkyl]₂-N—C(═O)—, and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆) alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6} N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; or
R^{Y1} and R^{Y2} taken together with the carbon atom to which they are attached form spiropyrrolidinyl or spiropiperidinyl, both of which are optionally N-substituted with a substituent selected from (C₁-C₆)alkyl, (C₁-C₆)alkyl-C(═O)—, [(C₁-C₆)alkyl]-C(═O)-(C₁-C₆)alkyl and aryl-(C═O)— wherein aryl is selected from phenyl and naphthyl;
R^{Y3} is hydrogen;
R^{Y4} is selected from hydroxy; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a1}R^{a2}N—C(═O)—, wherein R^{a1} R^{a2}, R^{a3} and R^{a4} are independently selected from hydrogen, (C₁-C₆)akyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6} R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and
R^{Y5}, R^{Y6} and R^{Y7} are independently selected from hydrogen; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; hetrocyclyl-(CH₂)ₘ₆— wherein n6 is an integer selected from 0, 1, 2, 3 and 4 and said heterocyclyl is four to eight membered containing one to three hetero atoms independently selected from nitrogen, oxygen and sulfur, wherein said heterocyclyl is optionally substituted with one to three substituents independently selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; and
hetroaryl-(CH₂)ₙ₇— wherein n7 is an integer selected from 0, 1, 2, 3 and 4 and said heteroaryl is five to ten membered containing one to three hetero atoms independently selected from nitrogen, oxygen and sulfur, wherein said heteroaryl is optionally substituted with one to three substituents independently selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; or
R^{Y6} and R^{Y7} taken together with the nitrogen atom to which they are attached form a four to eight heterocyclyl optionally containing, in addition to the nitrogen atom, one to two additional hetero atoms independently selected from nitrogen, oxygen and sulfur, and said heterocyclyl is optionally substituted with one substituent selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—;
said A is optionally substituted in the fused benzene rings with one to four substituents independently selected from halo; hydroxy; mercapto; phenyl; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and
Z is selected from C(═O); (CH₂)ₙ₈ wherein n8 is an integer selected from 0, 1 and 2; and CHR^{Z1} wherein
R^{Z1} is selected from carboxy; (C₁-C₆)alkoxy-C(═O)—; non-, mono- and di-substituted amino wherein the substituents are independently selected from (C₁-C₆)alkyl, [(C₁-C₆)alkyl]-C(═O)—, [(C₁-C₆)alkyl]-C(═O)—O— and [(C₁-C₆)alkyl]-SO₂—; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and
[C(═O)—NR^{Z11}R^{Z12}] wherein R^{Z11} and R^{Z12} are independently selected from hydrogen and (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—.

21. A compound according to claim 1 wherein
all R¹ are hydrogen
each R² is independently selected from hydrogen and halo;
X¹ is selected from (CH₂)ₙ₁ wherein n1 is an integer selected from 1, 2 and 3; O; NH; S; C(═O); SO₂; and N[(C₁-C₄)alkyl];
X² is selected from CH2; O; NH; S; C(═O); SO₂; and N[(C₁-C₄)alkyl]; or
X¹ and X² taken together form CH═CH;
W¹ and W² are independently selected from CR^{W1}R^{W2},
wherein
R^{W1} and R^{W2} are independently selected from hydrogen; halo; hydroxy; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; C(═O)-[(C₁-C₆)alkyl] wherein said (C₁-C₆)alkyl is optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; C(═O)—NR^{W11}R^{W12} wherein R^{W11} and R^{W12} are independently selected from hydrogen and (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}R^{a2} R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; NR^{W13}R^{W14} wherein R^{W13} and R^{W14} are independently selected from hydrogen and (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; aryl selected from phenyl and naphthyl; and four- to eight-membered heterocyclyl containing one to four hetero atoms independently selected from nitrogen, oxygen and sulfur;
A is AC wherein
Y^{d}, Y^{e} and Y^{f} are independently selected from C(═O); CR^{Y1}R^{Y2}; CR^{Y3} [C(═O)R^{Y4}]; CR^{Y3}[NR^{Y5} C(═O)R^{Y4}]; CR^{Y3}[C(═O)NR^{Y6} R^{Y7}]; CR^{Y3}[NR^{Y6}R^{Y7}]; O; S; SO₂; NH; N[(C₁-C₆)alkyl] wherein said (C₁-C₆)alkyl is optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a1}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; N—(CH₂)ₙ₃-heterocyclyl wherein n3 is an integer selected from 0, 1, 2 and 3, and said heterocyclyl contains from four to eight ring atoms one or two of which are independently selected from nitrogen, oxygen and sulfur; N—(CH₂)ₙ₄-aryl wherein n4 is an integer selected from 0, 1, 2 and 3, and said aryl is selected from phenyl and naphthyl; and N—(CH₂)ₙ₅-heteroaryl wherein n5 is an integer selected from 0, 1, 2 and 3, and said heteroaryl is a five to ten membered aromatic heterocyclyl containing from one to four hetero atoms independently selected from nitrogen, oxygen and sulfur;
R^{Y1} and R^{Y2} are independently selected from hydrogen; hydroxy; non-, mono- and di-substituted amino wherein the substituents are independently selected from (C₁-C₆)alkyl; [(C₁-C₆)alkyl]-C(═O)—; [(C₁-C₆)alkoxy]-C(═O)—; [(C₁-C₆)alkyl]-SO₂—; and four- to eight-membered heterocyclyl containing one to four hetero atoms independently selected from nitrogen, oxygen and sulfur, wherein said heterocyclyl is optionally substituted with one to three substituents independently selected from hydroxy, (C₁-C₆)alkyl, NH₂—C(O═)—, [(C₁-C₆)alkyl]-NH—C(═O)—, [(C₁-C₆)alkyl]₂-N—C(═O)—, and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a1}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; or
R^{Y1} and R^{Y2} taken together with the carbon atom to which they are attached form spiropyrrolidinyl or spiropiperidinyl, both of which are optionally N-substituted with a substituent selected from (C₁-C₆)alkyl, (C₁-C₆)alkyl-C(═O)—, [(C₁-C₆)alkyl]-C(═O)-(C₁-C₆)alkyl and aryl-(C═O)— wherein aryl is selected from phenyl and naphthyl;
R^{Y3} is hydrogen;
R^{Y4} is selected from hydroxy; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and
R^{Y5}, R^{Y6} and R^{Y7} are independently selected from hydrogen; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; hetrocyclyl-(CH₂)ₙ₆— wherein n6 is an integer selected from 0, 1, 2, 3 and 4 and said heterocyclyl is four to eight membered containing one to three hetero atoms independently selected from nitrogen, oxygen and sulfur, wherein said heterocyclyl is optionally substituted with one to three substituents independently selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; and hetroaryl-(CH₂)ₙ₇— wherein n7 is an integer selected from 0, 1, 2, 3 and 4 and said heteroaryl is five to ten membered containing one to three hetero atoms independently selected from nitrogen, oxygen and sulfur, wherein said heteroaryl is optionally substituted with one to three substituents independently selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; or
R^{Y6} and R^{Y7} taken together with the nitrogen atom to which they are attached form a four to eight heterocyclyl optionally containing, in addition to the nitrogen atom, one to two additional hetero atoms independently selected from nitrogen, oxygen and sulfur, and said heterocyclyl is optionally substituted with one substituent selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; and
said A is optionally substituted in the fused benzene rings with one to four substituents independently selected from halo; hydroxy; mercapto; phenyl; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3} R^{a4} N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and
Z is selected from C(═O); (CH₂)ₙ₈ wherein n8 is an integer selected from 0, 1 and 2; and CHR^{Z1} wherein
R^{Z1} is selected from carboxy; (C₁-C₆)alkoxy-C(═O)—; non-, mono- and di-substituted amino wherein the substituents are independently selected from (C₁-C₆)alkyl, [(C₁-C₆)alkyl]-C(═O)—, [(C₁-C₆)alkyl]-C(═O)—O— and [(C₁-C₆)alkyl]-SO₂—; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and
[C(═O)—NR^{Z11}R^{Z12}] wherein R^{Z11} and R^{Z12} are independently selected from hydrogen and (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4} N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—.

22. A compound according to claim 1 wherein
all R¹ are hydrogen
each R² is independently selected from hydrogen and halo;
X¹ and X² are each CH₂; or
X¹ and X² are taken together form CH═CH;
W¹ and W² are independently selected from CR^{W1}R^{W2},
wherein
R^{W1} and R^{W2} are independently selected from hydrogen; halo; hydroxy; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}, R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; C(═O)-[(C₁-C₆)alkyl] wherein said (C₁-C₆)alkyl is optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2} R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; C(═O)—NR^{W11}R^{W12} wherein R^{W11} and R^{W12} are independently selected from hydrogen and (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; NR^{W13} R^{W14} wherein R^{W13} and R^{W14} are independently selected from hydrogen and (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; aryl selected from phenyl and naphthyl; and four- to eight-membered heterocyclyl containing one to four hetero atoms independently selected from nitrogen, oxygen and sulfur;
A is AE wherein
Yⁱ, Y^{j}, Y^{k} and Y^{m} are independently selected from C(═O); CR^{Y1}R^{Y2}; CR^{Y3}[C(═O)R^{Y4}]; CR^{Y3}[NR^{Y5}C(═O)R^{Y4}]; CRY [C(═O)NR^{Y6} R^{Y7}]; CR^{Y3}[NR^{Y6} R^{Y7]}; O; S; SO₂; NH; N[(C₁-C₆)alkyl] wherein said (C₁-C₆)alkyl is optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; N—(CH₂)ₙ₃-heterocyclyl wherein n3 is an integer selected from 0, 1, 2 and 3, and said heterocyclyl contains from four to eight ring atoms one or two of which are independently selected from nitrogen, oxygen and sulfur; N—(CH₂)ₙ₄-aryl wherein n4 is an integer selected from 0, 1, 2 and 3, and said aryl is selected from phenyl and naphthyl; and N—(CH₂)ₙ₅-heteroaryl wherein n5 is an integer selected from 0, 1, 2 and 3, and said heteroaryl is a five to ten membered aromatic heterocyclyl containing from one to four hetero atoms independently selected from nitrogen, oxygen and sulfur;
R^{Y1} and R^{Y2} are independently selected from hydrogen; hydroxy; non-, mono- and di-substituted amino wherein the substituents are independently selected from (C₁-C₆)alkyl; [(C₁-C₆)alkyl]-C(═O)—; [(C₁-C₆)alkoxy]-C(═O)—; [(C₁-C₆)alkyl]-SO₂—; and four- to eight-membered heterocyclyl containing one to four hetero atoms independently selected from nitrogen, oxygen and sulfur, wherein said heterocyclyl is optionally substituted with one to three substituents independently selected from hydroxy, (C₁-C₆)alkyl, NH₂—C(O═)—, [(C₁-C₆)alkyl]-NH—C(═O)—, [(C₁-C₆)alkyl]₂-N—C(═O)—, and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; or
R^{Y1} and R^{Y2} taken together with the carbon atom to which they are attached form spiropyrrolidinyl or spiropiperidinyl, both of which are optionally N-substituted with a substituent selected from (C₁-C₆)alkyl, (C₁-C₆)alkyl-C(═O)—, [(C₁-C₆)alkyl]-C(═O)-(C₁-C₆)alkyl and aryl-(C═O)— wherein aryl is selected from phenyl and naphthyl;
R^{Y3} is hydrogen;
R^{Y4} is selected from hydroxy; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁C₆)alkyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and
R^{Y5}, R^{Y6} and R^{Y7} are independently selected from hydrogen; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; hetrocyclyl-(CH₂)ₙ₆— wherein n6 is an integer selected from 0, 1, 2, 3 and 4 and said heterocyclyl is four to eight membered containing one to three hetero atoms independently selected from nitrogen, oxygen and sulfur, wherein said heterocyclyl is optionally substituted with one to three substituents independently selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; and hetroaryl-(CH₂)ₙ₇— wherein n7 is an integer selected from 0, 1, 2, 3 and 4 and said heteroaryl is five to ten membered containing one to three hetero atoms independently selected from nitrogen, oxygen and sulfur, wherein said heteroaryl is optionally substituted with one to three substituents independently selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; or
R^{Y6} and R^{Y7} taken together with the nitrogen atom to which they are attached form a four to eight heterocyclyl optionally containing, in addition to the nitrogen atom, one to two additional hetero atoms independently selected from nitrogen, oxygen and sulfur, and said heterocyclyl is optionally substituted with one substituent selected from hydroxy; (C₁-C₆)alkyl; NH₂—C(O═)—; (C₁-C₆)alkyl-NH—C(═O)—; [(C₁-C₆)alkyl]₂-N—C(═O)—; and non-, mono- and di-substituted amino wherein the substituents are independently selected from (C₁-C₆)alkyl, [(C₁-C₆)alkyl]-C(═O)—, [(C₁-C₆)alkoxy]-C(═O)— and [(C₁-C₆)alkyl]-SO₂—; and
said A is optionally substituted in the fused benzene rings with one to four substituents independently selected from halo; hydroxy; mercapto; phenyl; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and (C₁-C₆)alkoxy optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a5}R^{a6}N— and R^{a7}R^{a8}N—C(═O)—, wherein R^{a5}, R^{a6}, R^{a7} and R^{a8} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and
Z is selected from C(═O); (CH₂)ₙ₈ wherein n8 is an integer selected from 0, 1 and 2; and
CHR^{Z1} wherein
R^{Z1} is selected from carboxy; (C₁-C₆)alkoxy-C(═O)—; non-, mono- and di-substituted amino wherein the substituents are independently selected from (C₁-C₆)alkyl, [(C₁-C₆)alkyl]-C(═O)—, [(C₁-C₆)alkyl]-C(═O)—O— and [(C₁-C₆)alkyl]-SO₂—; (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—; and [C(═O)—NR^{Z11}R^{Z12}] wherein R^{Z11} and R^{Z12} are independently selected from hydrogen and (C₁-C₆)alkyl optionally substituted with one to three substituents independently selected from halo, hydroxy, carboxy, [(C₁-C₆)alkyl]-C(═O)—, (C₁-C₆)alkoxy, [(C₁-C₆)alkoxy]-C(═O)—, R^{a1}R^{a2}N— and R^{a3}R^{a4}N—C(═O)—, wherein R^{a1}, R^{a2}, R^{a3} and R^{a4} are independently selected from hydrogen, (C₁-C₆)alkyl, [(C₁-C₆)alkyl]-C(═O)—, [(C₁-C₆)alkoxy]-C(═O)— and [(C₁-C₆)alkyl]-SO₂—.

23. A compound according to claim 1 selected from
2,3-dihydro-1′-{3-[2-(N-methylaminocarbonyl)indolin-1-yl]-3-oxopropyl} spiro[1H-indene-1,4′-piperidine];
2,3-dihydro-1′-[3-(2-N,N-dimethylaminocarbonylindolin-1-yl)-3-oxopropyl]spiro[1H-indene-1,4′-piperidine];
2,3-dihydro-1′-[3-(2-morpholinocarbonylindolin-1-yl)-3-oxopropyl]spiro[1H-indene-1,4′-piperidine];
2,3-dihydro-1′-[3-(2-carbamoylindolin-1-yl)-3-oxopropyl]spiro[1H-indene-1,4′-piperidine] hydrochloride;
2,3-dihydro-1′-{3-[2-(1-ethylprrolydin-3-yl)aminocarbonylindolin-1-yl]-3-oxopropyl} spiro[1H-indene-1,4′-piperidine];
2,3-dihydro-1′-{3-[2-(S)—(N,N-dimethylaminoethyl)aminocarbonylindolin-1-yl]-3-oxopropyl} spiro[1H-indene-1,4′-piperidine];
2,3-dihydro-1′-{3-[2-(S)-(2-hydroxyethyl)aminocarbonylindolin-1-yl]-3-oxopropyl}spiro[1H-indene-1,4′-piperidine];
2,3-dihydro-1′-{3-[2-(S)-(2-aminoethyl)aminocarbonylindolin-1-yl]-3-oxopropyl} spiro[1H-indene-1,4′-piperidine];
2,3-dihydro-1′-{3-[2-(S)-(2-acetamidoethyl)aminocarbonylindolin-1-yl]-3-oxopropyl} spiro[1H-indene-1,4′-piperidine];
2,3-dihydro-1′-{3-[2-(S)-(2-methanesulfonamidoethyl)aminocarbonylindolin-1-yl]-3-oxopropyl} spiro[1H-indene-1,4′-piperidine];
2,3-dihydro-1′-[3-(2-(S)-N-methylaminocarbonylindolin-1-yl)-3-oxopropyl]spiro [1H-indene-1,4′-piperidine];
2,3-dihydro-1′-[3-(2-(S)-N,N-dimethylaminocarbonylindolin-1-yl)-3-oxopropyl]spiro[1H-indene-1,4′-piperidine];
2,3-dihydro-1′-{3-[2-(S)-(4-morpholinecarbonyl)indolin-1-yl]-3-oxopropyl} spiro[1H-indene-1,4′-piperidine];
2,3-dihydro-1′-[3-(2-(S)-aminocarbonylindolin-1-yl)-3-oxopropyl]spiro[1H-indene-1,4′-piperidine];
2,3-dihydro-11′-[3-(2-methoxycarbonylindolin-1-yl)-3-oxopropyl]spiro[1H-indene-1,4′-piperidine];
2,3-dihydro-1′-[3-(indolin-1-yl)-3-oxopropyl]spiro[1H-indene-1,4′-piperidine];
2,3-dihydro-1′-[3-(2-(S)-methoxycarbonylindolin-1-yl)-3-oxopropyl]spiro[1H-indene-1,4′-piperidine];
2,3-dihydro-11′-indolyl-3-oxopropyl]spiro[1H-indene-1,4′-piperidine];
2,3-dihydro-1′-[3-(2-hydroxymethylindolin-1-yl)-3-oxopropyl]spiro[1H-indene-1,4′-piperidine];
2,3-dihydro-1′-[3-(2-methoxymethylindolin-1-yl)-3-oxopropyl]spiro[1H-indene-1,4′-piperidine];
2,3-dihydro-1′-[3-(benzimidazol-2-one-1-yl)propyl]spiro[1H-indene-1,4′-piperidine];
2,3-dihydro-1′-[3-(benzothiazol-2-one-1-yl)propyl]spiro[1H-indene-1,4′-piperidine];
2,3-dihydro-1′-[3-(2-oxo-1,3-benzoxazol-3(2H)-yl)propyl]spiro[1H-indene-1,4′-piperidine];
2,3-dihydro-1′-[3-(2-hydroxymethylbenzimidazol-1-yl)-3-oxopropyl]spiro[1H-indene-1,4′-piperidine];
2,3-dihydro-1′-[3-(3-ethylbenzimidazol-2-one-1-yl)propyl]spiro[1H-indene-1,4′-piperidine];
2,3-dihydro-1′-[3-(2-acetamidobenzimidazol-1-yl)propyl]spiro[1H-indene-1,4′-piperidine];
2,3-dihydro-1′-{3-[3-(2-hydroxyethyl)benzimidazol-2-one-1-yl)propyl} spiro[1H-indene-1,4′-piperidine];
2,3-dihydro-1′-{3-[3-(2-aminoethyl)benzimidazol-2-one-1-yl)propyl} spiro[1H-indene-1,4′-piperidine];
2,3-dihydro-1′-{3-[3-(2-acetamidoethyl)benzimidazol-2-one-1-yl)propyl} spiro[1H-indene-1,4′-piperidine];
2,3-dihydro-1′-[3-(2-oxo-3,4-dihydro-1 (2H)-quinolinyl)propyl]spiro[1H-indene-1,4′-piperidine];
2,3-dihydro-1′-[3-(3-methyl-2-oxo-3,4-dihydro-1 (2H)-quinazolinyl)propyl]spiro[1H-indene-1,4′-piperidine]; and
2,3-dihydro-1′-[3-oxo-3-(2,3,4,5-tetrahydro-1H-benzazepin-1-yl)propyl]spiro[1H-indene-1,4′-piperidine];
or a salt thereof.

24. A pharmaceutical composition comprising an effective amount of a compound of claim 1 and a pharmaceutically acceptable carrier for treating a disease or medical condition mediated by ORL1-receptor and its endogeneous ligand in a mammal including a human.

25. A method for treating a disease in a mammal including a human, wherein said disease is selected from; pain; eating disorders; anxiety and stress conditions; immune system diseases; locomotor disorder; memory loss, cognitive disorders and dementia; epilepsy or convulsion and symptoms associated therewith; anti-epileotic action; disruption of spatial memory; drug abuse; cardiovascular disorders; hypotension; bradycardia; stroke; renal disorders; water excretion, sodium ion excretion; syndrome of inappropriate secretion of antidiuretic hormone (SIADH); gastrointestinal disorders; airway disorders; metabolic disorders; cirrhosis with ascites; sexual dysfunctions; altered pulmonary function, comprising administering an effective amount of a compound of claim 1 to a mammal including a human, which suffers from such disease.
